(19) United States
(12) Patent Application Publication
(10) Pub. No.: US 2025/0063948 A1
(43) Pub. Date: Feb. 20, 2025

(54) HIGH EFFICIENCY AND LONG LIFETIME ORGANIC LIGHT-EMITTING DIODE

(71) Applicant: SFC CO., LTD., Cheongju-si (KR)

(72) Inventors: Kyung-Tae KIM, Cheongju-si (KR); Se-Jin LEE, Cheongju-si (KR); Si-In KIM, Cheongju-si (KR); Seok-Bae PARK, Cheongju-si (KR); Hee-Dae KIM, Cheongju-si (KR); Yeong-Tae CHOI, Cheongju-si (KR); Ji-Yung KIM, Cheongju-si (KR); Seung-Soo LEE, Cheongju-si (KR); Kyeong-Hyeon KIM, Cheongju-si (KR); Tae-Gyun LEE, Cheongju-si (KR); Joon-Ho KIM, Cheongju-si (KR)

(21) Appl. No.: 18/711,857

(22) PCT Filed: Nov. 25, 2022
(86) PCT No.: PCT/KR2022/018823

§ 371 Date: May 20, 2024

(30) Foreign Application Priority Data
Nov. 26, 2021 (KR) 10-2021-0166060

(51) Int. Cl. H10K 85/60; C09K 11/02; H10K 50/12; H10K 85/20; H10K 85/30; H10K 85/40

(52) U.S. Cl. CPC H10K 85/6574; C09K 11/02; H10K 50/12; H10K 85/20; H10K 85/30; H10K 85/40; H10K 85/622; H10K 85/633; H10K 85/654; H10K 85/656; H10K 85/6576; H10K 85/658

(57) ABSTRACT

The present invention relates to an organic light-emitting diode in which a light-emitting layer comprises a compound represented by [chemical formula A] as a host and comprises a compound represented by [chemical formula 3], [chemical formula 4], [chemical formula 3-1] to [chemical formula 4-3] as a dopant, wherein [chemical formula 3], [chemical formula 4], [chemical formula 3-1] to [chemical formula 4-3] are the same as those described in the detailed description of the invention.

### TECHNICAL FIELD

The present disclosure relates to an organic light-emitting diode with high efficiency and longevity and, more specifically, to an organic light-emitting diode that employs compounds having specific structures as host and dopant materials in a light-emitting layer thereof, thereby exhibiting high emission efficiency and longevity.

### BACKGROUND ART

Organic light-emitting diodes (OLEDs), based on self-luminescence, are used to create digital displays with the advantage of having a wide viewing angle and being able to be made thinner and lighter than liquid crystal displays. In addition, an OLED display exhibits a very fast response time. Accordingly, OLEDs find applications in the full color display field or the illumination field.

In general, the term “organic light-emitting phenomenon” refers to a phenomenon in which electrical energy is converted to light energy by means of an organic material. An organic light-emitting diode using the organic light-emitting phenomenon has a structure usually including an anode, a cathode, and an organic material layer interposed therebetween. In this regard, the organic material layer can have, for the most part, a multilayer structure consisting of different materials, for example, a hole injection layer, a hole transport layer, a light-emitting layer, an electron transport layer, and an electron injection layer in order to enhance the efficiency and stability of the organic light-emitting diode. In the organic light-emitting diode having such a structure, application of a voltage between the two electrodes injects a hole from the anode and an electron from the cathode to the organic layer. In the luminescent zone, the hole and the electron recombine to produce an exciton. When the exciton returns to the ground state from the excited state, the molecule of the organic layer emits light. Such an organic light-emitting diode is known to have characteristics such as self-luminescence, high luminance, high efficiency, low driving voltage, a wide viewing angle, high contrast, and high-speed response.

Materials used as organic layers in OLEDs may be divided according to functions into luminescent materials and charge transport materials, for example, a hole injection material, a hole transport material, an electron transport material, and an electron injection material. As for the luminescent materials, there are two main families of OLED: those based on small molecules and those employing polymers. The light-emitting mechanism forms the basis of classification of luminescent materials as fluorescent and phosphorescent materials, which use excitons in singlet and triplet states, respectively.

When a single material is employed as the luminescent material, intermolecular actions cause the maximum luminescence wavelength to shift toward a longer wavelength, resulting in a reduction in color purity and luminous efficiency due to light attenuation. In this regard, a host-dopant system may be used as a luminescent material so as to increase the color purity and the luminous efficiency through energy transfer.

This is based on the principle whereby, when a dopant which is smaller in energy band gap than a host forming a light-emitting layer is added in a small amount to the light-emitting layer, excitons are generated from the light-emitting layer and transported to the dopant, emitting light at high efficiency. Here, light with desired wavelengths can be obtained depending on the kind of the dopant because the wavelength of the host moves to the wavelength range of the dopant.

For use as host compounds in a light-emitting layer, heterocyclic compounds have been recently studied. With regard to related art, reference may be made to Korean Patent Publication No. 10-2016-0089693 A (Jul. 28, 2016), which discloses a compound structured to have a dibenzofuran ring moiety bonded to an anthracene ring, and an organic light-emitting diode including same. In addition, Korean Patent Publication No. 10-2017-0055743 A (May 22, 2017) discloses a compound in which an aryl substituent or a heteroaryl substituent is bonded to a fused fluorene ring bearing a heteroatom such as oxygen, nitrogen, sulfur, etc., and an organic light-emitting diode including same.

Despites a variety of types of compounds prepared for use in light emitting layers in organic light-emitting diodes including the related arts, there is still a continuing need to develop an organic layer material that allows the organic light-emitting diodes to be driven with high efficiency and longevity.

### DISCLOSURE

### Technical Problem

Therefore, the present disclosure aims to provide an organic light-emitting diode (OLED), characterized by high efficiency and longevity, which employs a host material and a dopant material having respective specific structures.

### Technical Solution

To accomplish the technical subject, the present disclosure provides an organic light-emitting diode including: a first electrode: a second electrode; and a light-emitting layer interposed between the first electrode and the second electrode, wherein the light-emitting layer contains a host and a dopant, the host including at least one of the anthracene compounds represented by the following Chemical Formula A, the dopant including at least one of the polycyclic compounds represented by the following Chemical Formula 3 or 4:

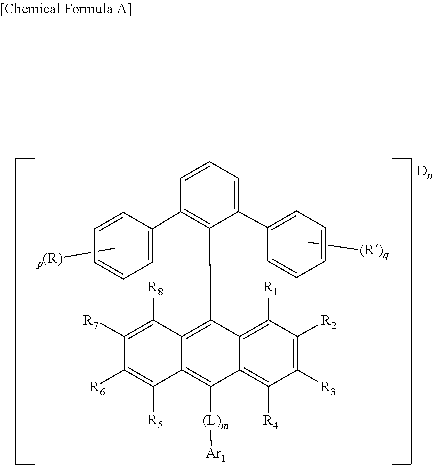

wherein,
R, R′, and R₁ to R₈, which are same or different, are each independently a hydrogen atom, a deuterium atom, a substituted or unsubstituted alkyl of 1 to 30 carbon atoms, a substituted or unsubstituted alkenyl of 2 to 30 carbon atoms, a substituted or unsubstituted aryl of 6 to 50 carbon atoms, a substituted or unsubstituted cycloalkyl of 3 to 30 carbon atoms, a substituted or unsubstituted heterocycloalkyl of 3 to 30 carbon atoms, a substituted or unsubstituted heteroaryl of 2 to 50 carbon atoms, a substituted or unsubstituted aromatic hydrocarbon ring-fused cycloalkyl of 7 to 30 carbon atoms, a substituted or unsubstituted heteroaromatic ring-fused cycloalkyl of 5 to 30 carbon atoms, a substituted or unsubstituted aromatic hydrocarbon ring-fused heterocycloalkyl of 6 to 30 carbon atoms, a substituted or unsubstituted aliphatic hydrocarbon ring-fused aryl of 7 to 30 carbon atoms, a substituted or unsubstituted aliphatic hydrocarbon ring-fused heteroaryl of 5 to 30 carbon atoms, a substituted or unsubstituted heteroaliphatic ring-fused aryl of 6 to 30 carbon atoms, a substituted or unsubstituted heteroaliphatic ring-fused heteroaryl of 5 to 30 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted amine of 0 to 30 carbon atoms, a substituted or unsubstituted silyl of 0 to 30 carbon atoms, a substituted or unsubstituted germanium of 0 to 30 carbon atoms, a nitro, a cyano, and a halogen,
L, which functions as a linker, is any one selected from a single bond, a substituted or unsubstituted arylene of 6 to 50 carbon atoms, a substituted or unsubstituted heteroarylene of 2 to 50 carbon atoms, a substituted or unsubstituted aromatic hydrocarbon ring-fused cycloalkylene of 7 to 30 carbon atoms, and a substituted or unsubstituted aliphatic hydrocarbon ring-fused arylene of 8 to 30 carbon atoms,
m is an integer of 1 to 3, wherein when m is 2 or higher, the corresponding L's are same or different,
Ar₁ is any one selected from a substituted or unsubstituted aryl of 6 to 50 carbon atoms, a substituted or unsubstituted heteroaryl of 2 to 50 carbon atoms, a substituted or unsubstituted cycloalkyl of 3 to 30 carbon atoms, a substituted or unsubstituted heterocycloalkyl of 3 to 30 carbon atoms, a substituted or unsubstituted aromatic hydrocarbon ring-fused cycloalkyl of 7 to 30 carbon atoms, a substituted or unsubstituted heteroaromatic ring-fused cycloalkyl of 5 to 30 carbon atoms, a substituted or unsubstituted aromatic hydrocarbon ring-fused heterocycloalkyl of 6 to 30 carbon atoms, a substituted or unsubstituted aliphatic hydrocarbon ring-fused aryl of 7 to 30 carbon atoms, a substituted or unsubstituted aliphatic hydrocarbon ring-fused heteroaryl of 5 to 30 carbon atoms, a substituted or unsubstituted heteroaliphatic ring-fused aryl of 6 to 30 carbon atoms, and a substituted or unsubstituted heteroaliphatic ring-fused heteroaryl of 5 to 30 carbon atoms,
Dₙ stands for a number (n) of deuterium atoms substituted for hydrogen atoms in Chemical Formula A,
n is an integer of 0 to 60,
p and q, which are same or different, are each independently an integer of 1 to 5, where when p or q is 2 or higher, the corresponding R or R′ radicals are same or different,
the plural R radicals may be connected to each other to additionally form an aliphatic or aromatic mono- or polycyclic ring, and
the plural R′ radicals may be connected to each other to additionally form an aliphatic or aromatic mono- or polycyclic ring;

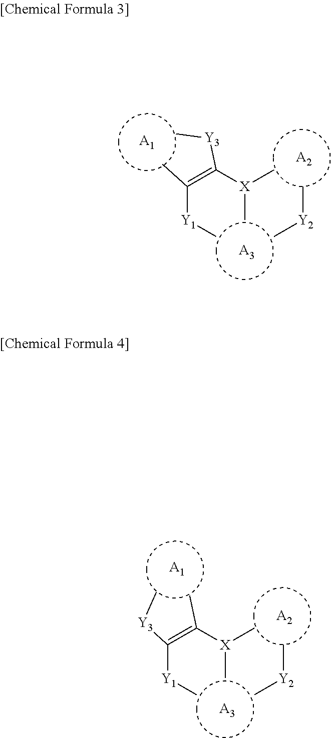

wherein,
X is B, P═O, P=S, or Al,
Y₁ and Y₂, which are same or different, are each independently any one selected from NR₁₁, O, S, CR₁₂R₁₃, SiR₁₄R₁₅, and GeR₁₆R₁₇,
Y₃ is O, S, or NR₁₀,
A₁ to A₃, which are same or different, are each independently a substituted or unsubstituted aromatic hydrocarbon ring of 6 to 50 carbon atoms, a substituted or unsubstituted aliphatic hydrocarbon ring-fused aromatic hydrocarbon ring of 6 to 50 carbon atoms, a substituted or unsubstituted heteroaromatic ring of 2 to 50 carbon atoms, and a substituted or unsubstituted aliphatic hydrocarbon ring-fused heteroaromatic ring of 2 to 50 carbon atoms,
R₁₀ to R₁₇, which are same or different, are each independently any one selected from a hydrogen atom, a deuterium atom, a substituted or unsubstituted alkyl of 1 to 30 carbon atoms, a substituted or unsubstituted alkenyl of 2 to 30 carbon atoms, a substituted or unsubstituted aryl of 6 to 50 carbon atoms, a substituted or unsubstituted cycloalkyl of 3 to 30 carbon atoms, a substituted or unsubstituted heterocycloalkyl of 3 to 30 carbon atoms, a substituted or unsubstituted heteroaryl of 2 to 50 carbon atoms, a substituted or unsubstituted aromatic hydrocarbon ring-fused cycloalkyl of 7 to 30 carbon atoms, a substituted or unsubstituted heteroaromatic ring-fused cycloalkyl of 5 to 30 carbon atoms, a substituted or unsubstituted aromatic hydrocarbon ring-fused heterocycloalkyl of 6 to 30 carbon atoms, a substituted or unsubstituted aliphatic hydrocarbon ring-fused aryl of 7 to 30 carbon atoms, a substituted or unsubstituted aliphatic hydrocarbon ring-fused heteroaryl of 5 to 30 carbon atoms, a substituted or unsubstituted heteroaliphatic ring-fused aryl of 6 to 30 carbon atoms, a substituted or unsubstituted heteroaliphatic ring-fused heteroaryl of 5 to 30 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted amine of 0 to 30 carbon atoms, a substituted or unsubstituted silyl of 0 to 30 carbon atoms, a substituted or unsubstituted germanium of 0 to 30 carbon atoms, a nitro, a cyano, and a halogen,
at least one of R₁₀ to R₁₇ may be connected to any one of the A₁ to A₃ ring moiety to additionally form an aliphatic or aromatic mono- or polycyclic ring,
a linkage may be formed between R₁₂ and R₁₃, between R₁₄ and R₁₅, and/or between R₁₆ and R₁₇ to additionally form an aliphatic or aromatic mono- or polycyclic ring,
   wherein the term “substituted” in the expression “substituted or unsubstituted” used for the compounds of Chemical Formulas A, 3, and 4 means having at least one substituent selected from the group consisting of a deuterium atom, a cyano, a halogen, a hydroxy, a nitro, alkyl of 1 to 30 carbon atoms, a halogenated alkyl of 1 to 30 carbon atoms, an alkenyl of 2 to 30 carbon atoms, an alkynyl of 2 to 30 carbon atoms, a cycloalkyl of 3 to 30 carbon atoms, a heteroalkyl of 1 to 30 carbon atoms, an aryl of 6 to 30 carbon atoms, an arylalkyl of 7 to 30 carbon atoms, an alkylaryl of 7 to 30 carbon atoms, a heteroaryl of 2 to 30 carbon atoms, a heteroarylalkyl of 3 to 30 carbon atoms, an alkylheteroaryl of 3 to 30 carbon atoms, an alkoxy of 1 to 30 carbon atoms, an aromatic hydrocarbon ring-fused cycloalkyl of 7 to 30 carbon atoms, a heteroaromatic ring-fused cycloalkyl of 5 to 30 carbon atoms, an aromatic hydrocarbon ring-fused heterocycloalkyl of 6 to 30 carbon atoms, an aliphatic hydrocarbon ring-fused aryl of 7 to 30 carbon atoms, an aliphatic hydrocarbon ring-fused heteroaryl of 5 to 30 carbon atoms, a substituted or unsubstituted heteroaliphatic ring-fused aryl of 6 to 30 carbon atoms, a substituted or unsubstituted heteroaliphatic ring-fused heteroaryl of 5 to 30 carbon atoms, an amine of 1 to 40 carbon atoms, a silyl of 1 to 40 carbon atoms, a germanium of 1 to 40 carbon atoms, an aryloxy of 6 to 30 carbon atoms, and an arylthionyl of 6 to 30 carbon atoms, with at least one hydrogen atom on the substituent being substitutable with a deuterium atom.

Also, the present disclosure provides an organic light-emitting diode including: a first electrode: a second electrode; and a light-emitting layer interposed between the first electrode and the second electrode, wherein the light-emitting layer contains a host and a dopant, the host including at least one of the anthracene compounds represented by the following Chemical Formula A, the dopant including at least one of the polycyclic compounds represented by any one of the following Chemical Formula 3-1 to 4-3:

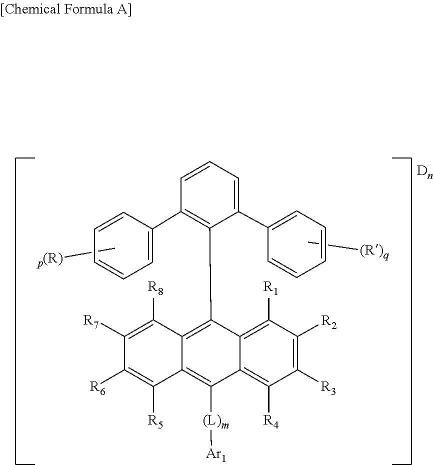

wherein
R, R′, and R₁ to R₈, which are same or different, are each independently any one selected from a hydrogen atom, a deuterium atom, a substituted or unsubstituted alkyl of 1 to 30 carbon atoms, a substituted or unsubstituted alkenyl of 2 to 30 carbon atoms, a substituted or unsubstituted aryl of 6 to 50 carbon atoms, a substituted or unsubstituted cycloalkyl of 3 to 30 carbon atoms, a substituted or unsubstituted heterocycloalkyl of 3 to 30 carbon atoms, a substituted or unsubstituted heteroaryl of 2 to 50 carbon atoms, a substituted or unsubstituted aromatic hydrocarbon ring-fused cycloalkyl of 7 to 30 carbon atoms, a substituted or unsubstituted heteroaromatic ring-fused cycloalkyl of 5 to 30 carbon atoms, a substituted or unsubstituted aromatic hydrocarbon ring-fused heterocycloalkyl of 6 to 30 carbon atoms, a substituted or unsubstituted aliphatic hydrocarbon ring-fused aryl of 7 to 30 carbon atoms, a substituted or unsubstituted aliphatic hydrocarbon ring-fused heteroaryl of 5 to 30 carbon atoms, a substituted or unsubstituted heteroaliphatic ring-fused aryl of 6 to 30 carbon atoms, a substituted or unsubstituted heteroaliphatic ring-fused heteroaryl of 5 to 30 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted amine of 0 to 30 carbon atoms, a substituted or unsubstituted silyl of 0 to 30 carbon atoms, a substituted or unsubstituted germanium of 0 to 30 carbon atoms, a nitro, a cyano, and a halogen,
L, which functions as a linker, is any one selected from a single bond, a substituted or unsubstituted arylene of 6 to 50 carbon atoms, a substituted or unsubstituted heteroarylene of 2 to 50 carbon atoms, a substituted or unsubstituted aromatic hydrocarbon ring-fused cycloalkylene of 7 to 30 carbon atoms, and a substituted or unsubstituted aliphatic hydrocarbon ring-fused arylene of 8 to 30 carbon atoms,
m is an integer of 1 to 3, wherein when m is 2 or higher, the corresponding L's are same or different,
Ar₁ is any one selected from a substituted or unsubstituted aryl of 6 to 50 carbon atoms, a substituted or unsubstituted heteroaryl of 2 to 50 carbon atoms, a substituted or unsubstituted cycloalkyl of 3 to 30 carbon atoms, a substituted or unsubstituted heterocycloalkyl of 3 to 30 carbon atoms, a substituted or unsubstituted aromatic hydrocarbon ring-fused cycloalkyl of 7 to 30 carbon atoms, a substituted or unsubstituted heteroaromatic ring-fused cycloalkyl of 5 to 30 carbon atoms, a substituted or unsubstituted aromatic hydrocarbon ring-fused heterocycloalkyl of 6 to 30 carbon atoms, a substituted or unsubstituted aliphatic hydrocarbon ring-fused aryl of 7 to 30 carbon atoms, a substituted or unsubstituted aliphatic hydrocarbon ring-fused heteroaryl of 5 to 30 carbon atoms, a substituted or unsubstituted heteroaliphatic ring-fused aryl of 6 to 30 carbon atoms, and a substituted or unsubstituted heteroaliphatic ring-fused heteroaryl of 5 to 30 carbon atoms,
Dₙ stands for a number (n) of deuterium atoms substituted for hydrogen atoms in Chemical Formula A,
n is an integer of 0 to 60,
p and q, which are same or different, are each independently an integer of 1 to 5, where when p or q is 2 or higher, the corresponding R or R′ radicals are same or different,
the plural R radicals may be connected to each other to additionally form an aliphatic or aromatic mono- or polycyclic ring, and
the plural R′ radicals may be connected to each other to additionally form an aliphatic or aromatic mono- or polycyclic ring;

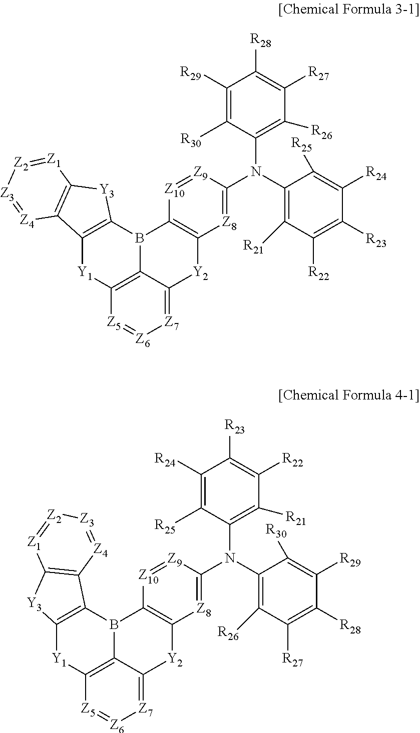

wherein
Z₁ to Z₁₀, which are same or different, are each independently CR₃₁ or N, when at least two of Z₁ to Z₁₀ are each CR₃₁, the corresponding CR₃₁'s are same or different,
when at least two of Z₁ to Z₁₀ are each CR₃₁, the corresponding R₃₁'s are connected to additionally form an aliphatic or aromatic mono- or polycyclic ring,
R₂₁ to R₃₁, which are same or different, are each independently any one selected from a hydrogen atom, a deuterium atom, an unsubstituted alkyl of 1 to 24 carbon atoms, an unsubstituted alkynyl of 2 to 24 carbon atoms, an unsubstituted alkenyl of 2 to 24 carbon atoms, an unsubstituted aryl of 6 to 24 carbon atoms, an unsubstituted alkylaryl of 7 to 24 carbon atoms, an unsubstituted cycloalkyl of 3 to 24 carbon atoms, an unsubstituted heterocycloalkyl of 3 to 24 carbon atoms, an unsubstituted heteroaryl of 2 to 24 carbon atoms, an unsubstituted alkylheteroaryl of 3 to 24 carbon atoms, an unsubstituted aromatic hydrocarbon ring-fused cycloalkyl of 7 to 24 carbon atoms, an unsubstituted heteroaromatic ring-fused cycloalkyl of 5 to 24 carbon atoms, an unsubstituted aromatic hydrocarbon ring-fused heterocycloalkyl of 6 to 24 carbon atoms, an unsubstituted aliphatic hydrocarbon ring-fused aryl of 7 to 24 carbon atoms, an unsubstituted aliphatic hydrocarbon ring-fused heteroaryl of 5 to 24 carbon atoms, an unsubstituted heteroaliphatic ring-fused aryl of 6 to 24 carbon atoms, an unsubstituted heteroaliphatic ring-fused heteroaryl of 5 to 24 carbon atoms, an unsubstituted alkoxy of 1 to 24 carbon atoms, an unsubstituted aryloxy of 6 to 24 carbon atoms, an unsubstituted alkylthioxy of 1 to 24 carbon atoms, an unsubstituted arylthioxy of 5 to 24 carbon atoms, an unsubstituted amine of 1 to 24 carbon atoms, an unsubstituted silyl of 1 to 24 carbon atoms, an unsubstituted germanium of 1 to 24 carbon atoms, a nitro, a cyano, and a halogen,
R₂₁ to R₃₁ may have at least one deuterium atom instead of a hydrogen atom,
adjacent radicals among R₂₁ to R₃₀ may be connected to each other to additionally form an aliphatic or aromatic mono- or polycyclic ring,
when at least one of Z₁ to Z₁₀ is CR₃₁, at least one of R₂₁, R₂₅, R₂₆, and R₃₀ may be connected to the corresponding CR₃₁ to additionally form an aliphatic or aromatic mono- or polycyclic ring,
Y₁ to Y₃, which are same or different, are each independently as defined in Chemical Formulas 3 and 4 or a linker represented by the following Structural Formula A,
   with a proviso that at least one of Y₁ and Y₂ is a linker represented by the following Structural Formula A:

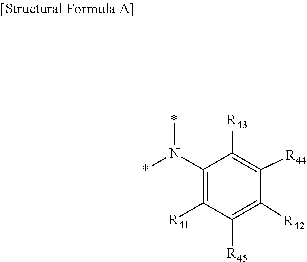

wherein,
R₄₁ to R₄₅, which are same or different, are each independently any one selected from a substituted or unsubstituted alkyl of 1 to 30 carbon atoms, a substituted or unsubstituted alkynyl of 2 to 30 carbon atoms, a substituted or unsubstituted alkenyl of 2 to 30 carbon atoms, a substituted or unsubstituted aryl of 6 to 50 carbon atoms, a substituted or unsubstituted cycloalkyl of 3 to 30 carbon atoms, a substituted or unsubstituted cycloalkenyl of 3 to 30 carbon atoms, a substituted or unsubstituted heterocycloalkyl of 1 to 30 carbon atoms, a substituted or unsubstituted heteroaryl of 2 to 50 carbon atoms, a substituted or unsubstituted aromatic hydrocarbon ring-fused cycloalkyl of 7 to 30 carbon atoms, a substituted or unsubstituted heteroaromatic ring-fused cycloalkyl of 5 to 30 carbon atoms, a substituted or unsubstituted aromatic hydrocarbon ring-fused heterocycloalkyl of 6 to 30 carbon atoms, a substituted or unsubstituted aliphatic hydrocarbon ring-fused aryl of 7 to 30 carbon atoms, a substituted or unsubstituted aliphatic hydrocarbon ring-fused heteroaryl of 5 to 30 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted amine of 0 to 30 carbon atoms, a substituted or unsubstituted silyl of 0 to 30 carbon atoms, a substituted or unsubstituted germanium of 0 to 30 carbon atoms, a nitro, a cyano, and a halogen,
at least one hydrogen atom in R₄₁ to R₄₅ is substitutable with a deuterium atom,
R₄₁ to R₄₅ may be connected to an adjacent substituent to additionally form an aliphatic or aromatic mono- or polycyclic ring;

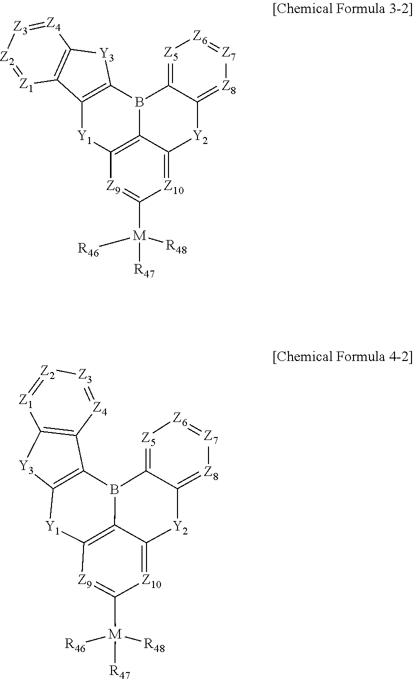

Y₁ to Y₃, which are same or different, are each independently as defined in Chemical Formulas 3 and 4,
Z₁ to Z₁₀, which are same or different, are each independently CR₃₁ or N,
when at least two of Z₁ to Z₁₀ are each CR₃₁, the corresponding CR₃₁'s are same or different,
when at least two of Z₁ to Z₈ are each CR₃₁, the corresponding R₃₁'s may be connected to each other to additionally form an aliphatic or aromatic mono- or polycyclic ring,
M is Si or Ge,
R₃₁ and R₄₆ to R₄₈, which are same or different, are each independently any one selected from a hydrogen atom, a deuterium atom, a substituted or unsubstituted alkyl of 1 to 30 carbon atoms, a substituted or unsubstituted alkynyl of 2 to 30 carbon atoms, a substituted or unsubstituted alkenyl of 2 to 30 carbon atoms, a substituted or unsubstituted aryl of 6 to 50 carbon atoms, a substituted or unsubstituted cycloalkyl of 3 to 30 carbon atoms, a substituted or unsubstituted cycloalkenyl of 3 to 30 carbon atoms, a substituted or unsubstituted heterocycloalkyl of 1 to 30 carbon atoms, a substituted or unsubstituted heteroaryl of 2 to 50 carbon atoms, a substituted or unsubstituted aromatic hydrocarbon ring-fused cycloalkyl of 7 to 30 carbon atoms, a substituted or unsubstituted heteroaromatic ring-fused cycloalkyl of 5 to 30 carbon atoms, a substituted or unsubstituted aromatic hydrocarbon ring-fused heterocycloalkyl of 6 to 30 carbon atoms, a substituted or unsubstituted aliphatic hydrocarbon ring-fused aryl of 7 to 30 carbon atoms, a substituted or unsubstituted aliphatic hydrocarbon ring-fused heteroaryl of 5 to 30 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted amine of 0 to 30 carbon atoms, a substituted or unsubstituted silyl of 0 to 30 carbon atoms, a substituted or unsubstituted germanium of 0 to 30 carbon atoms, a nitro, a cyano, and a halogen
at least one hydrogen atom in R₄₆ to R₄₀ is substitutable with a deuterium atom, and
R₄₆ to R₄₀ may each be connected to additionally form an aliphatic or aromatic mono- or polycyclic ring;

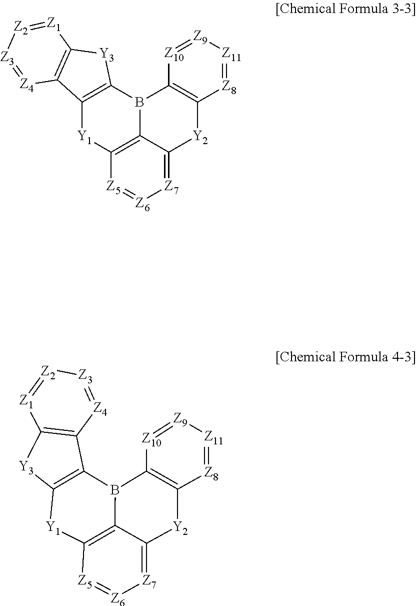

wherein,
Z₁ to Z₁₁, which are same or different, are each independently CR₃₁ or N,
when at least two of Z₁ to Z₁₁ are each CR₃₁, the corresponding CR₃₁'s are same or different,
when at least two of Z₁ to Z₁₁ are each CR₃₁, the corresponding R₃₁'s may be connected to each other to additionally form an aliphatic or aromatic mono- or polycyclic ring,
Y₁ to Y₃, which are same or different, are each independently as defined in Chemical Formulas 3 and 4, or a linker represented by the following Structural Formula B,
with a proviso that at least one of Y₁ and Y₂ is a linker represented by the following Structural Formula B:

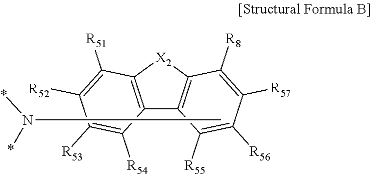

in Structural Formula B and Chemical Formulas 3-3 and 4-3,
X₂ is O or S,
R₃₁ and R₅₁ to R₅₈, which are same or different, are each independently any one selected from a hydrogen atom, a deuterium atom, a substituted or unsubstituted alkyl of 1 to 30 carbon atoms, a substituted or unsubstituted alkynyl of 2 to 30 carbon atoms, a substituted or unsubstituted alkenyl of 2 to 30 carbon atoms, a substituted or unsubstituted aryl of 6 to 50 carbon atoms, a substituted or unsubstituted cycloalkyl of 3 to 30 carbon atoms, a substituted or unsubstituted cycloalkenyl of 3 to 30 carbon atoms, a substituted or unsubstituted heterocycloalkyl of 1 to 30 carbon atoms, a substituted or unsubstituted heteroaryl of 2 to 50 carbon atoms, a substituted or unsubstituted aromatic hydrocarbon ring-fused cycloalkyl of 7 to 30 carbon atoms, a substituted or unsubstituted heteroaromatic ring-fused cycloalkyl of 5 to 30 carbon atoms, a substituted or unsubstituted aromatic hydrocarbon ring-fused heterocycloalkyl of 6 to 30 carbon atoms, a substituted or unsubstituted aliphatic hydrocarbon ring-fused aryl of 7 to 30 carbon atoms, a substituted or unsubstituted aliphatic hydrocarbon ring-fused heteroaryl of 5 to 30 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted amine of 0 to 30 carbon atoms, a substituted or unsubstituted silyl of 0 to 30 carbon atoms, a substituted or unsubstituted germanium of 0 to 30 carbon atoms, a nitro, a cyano, and a halogen,
at least one hydrogen atom in R₅₁ to R₅₈ is substitutable with a deuterium atom,
any one of R₅₁ to R₅₈ is a single bond linking to the nitrogen atom,
adjacent radicals among R₅₁ to R₅₈, except for the single bond, may be connected to each other to additionally form an aliphatic or aromatic mono- or polycyclic ring,
   wherein the term “substituted” in the expression “substituted or unsubstituted” used for compounds of Chemical Formulas A and 3-1 to 4-3 means having at least one substituent selected from the group consisting of a deuterium, a cyano, a halogen, a hydroxy, a nitro, alkyl of 1 to 30 carbon atoms, a halogenated alkyl of 1 to 30 carbon atoms, an alkenyl of 2 to 30 carbon atoms, an alkynyl of 2 to 30 carbon atoms, a cycloalkyl of 3 to 30 carbon atoms, a heteroalkyl of 1 to 30 carbon atoms, an aryl of 6 to 30 carbon atoms, an arylalkyl of 7 to 30 carbon atoms, an alkylaryl of 7 to 30 carbon atoms, a heteroaryl of 2 to 30 carbon atoms, a heteroarylalkyl of 3 to 30 carbon atoms, an alkylheteroaryl of 3 to 30 carbon atoms, an alkoxy of 1 to 30 carbon atoms, an aromatic hydrocarbon ring-fused cycloalkyl of 7 to 30 carbon atoms, heteroaromatic ring-fused cycloalkyl of 5 to 30 carbon atoms, aromatic hydrocarbon ring-fused heterocycloalkyl of 6 to 30 carbon atoms, an aliphatic hydrocarbon ring-fused aryl of 7 to 30 carbon atoms, an aliphatic hydrocarbon ring-fused heteroaryl of 5 to 30 carbon atoms, a substituted or unsubstituted heteroaliphatic ring-fused aryl of 6 to 30 carbon atoms, a substituted or unsubstituted heteroaliphatic ring-fused heteroaryl of 5 to 30 carbon atoms, an amine of 1 to 40 carbon atoms, a silyl of 1 to 40 carbon atoms, a germanium of 1 to 40 carbon atoms aryloxy of 6 to 30 carbon atoms, and an arylthionyl of 6 to 30 carbon atoms, with at least one hydrogen atom in each substituent being substitutable with a deuterium atom.

### Advantageous Effects

With a combination of the host and dopant materials in the light-emitting layer, the organic light-emitting diode according to the present disclosure exhibits higher efficiency and longevity compared to conventional organic light-emitting diodes.

### DESCRIPTION OF DRAWINGS

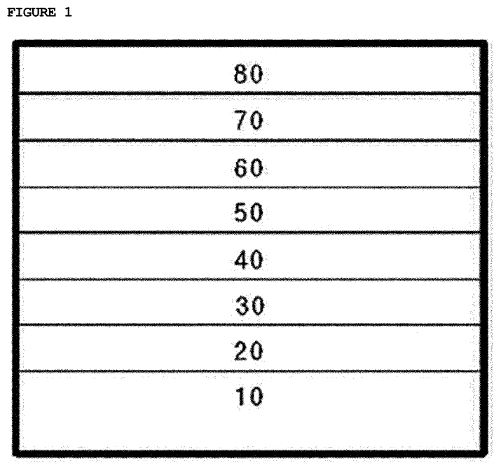

The FIG. 1 is a schematic diagram of an OLED according to some embodiments of the present disclosure.

### MODE FOR INVENTION

Below, a detailed description will be given of the present disclosure. In each drawing of the present disclosure, sizes or scales of components may be enlarged or reduced from their actual sizes or scales for better illustration, and known components may not be depicted therein to clearly show features of the present disclosure. Therefore, the present disclosure is not limited to the drawings. When describing the principle of the embodiments of the present disclosure in detail, details of well-known functions and features may be omitted to avoid unnecessarily obscuring the presented embodiments.

In the drawing, for convenience of description, sizes of components may be exaggerated for clarity. For example, since sizes and thicknesses of components in drawings are arbitrarily shown for convenience of description, the sizes and thicknesses are not limited thereto. Furthermore, throughout the description, the terms “on” and “over” are used to refer to the relative positioning, and mean not only that one component or layer is directly disposed on another component or layer but also that one component or layer is indirectly disposed on another component or layer with a further component or layer being interposed therebetween. Also, spatially relative terms, such as “below”, “beneath”, “lower”, and “between” may be used herein for ease of description to refer to the relative positioning.

Throughout the specification, when a portion may “include” a certain constituent element, unless explicitly described to the contrary, it may not be construed to exclude another constituent element but may be construed to further include other constituent elements. Further, throughout the specification, the word “on” means positioning on or below the object portion, but does not essentially mean positioning on the lower side of the object portion based on a gravity direction.

In another embodiment, the organic light-emitting diode according to the present disclosure includes: a first electrode; a second electrode facing the first electrode; and a light-emitting layer interposed between the first electrode and the second electrode, wherein the light-emitting layer contains a host and a dopant, the host including at least one of the anthracene compounds represented by Chemical Formula A, the dopant including at least one of the polycyclic compounds represented Chemical Formula 3 or 4.

In another embodiment, the organic light-emitting diode according to the present disclosure includes: a first electrode; a second electrode facing the first electrode; and a light-emitting layer interposed between the first electrode and the second electrode, wherein the light-emitting layer contains a host and a dopant, the host including at least one of the anthracene compounds represented by Chemical Formula A, the dopant including at least one of the polycyclic compounds represented Chemical Formula 3-1 to Chemical Formula 4-3.

The expression indicating the number of carbon atoms, such as “a substituted or unsubstituted alkyl of 1 to 30 carbon atoms”, “a substituted or unsubstituted aryl of 5 to 50 carbon atoms”, etc. means the total number of carbon atoms of, for example, the alkyl or aryl radical or moiety alone, exclusive of the number of carbon atoms of substituents attached thereto. For instance, a phenyl group with a butyl at the para position falls within the scope of an aryl of 6 carbon atoms, even though it is substituted with a butyl radical of 4 carbon atoms

As used herein, the term “aryl” means an organic radical derived from an aromatic hydrocarbon by removing one hydrogen that is bonded to the aromatic hydrocarbon. The aromatic system may include a fused ring that is formed by adjacent substituents on the aryl radical.

Concrete examples of the aryl include phenyl, o-biphenyl, m-biphenyl, p-biphenyl, o-terphenyl, m-terphenyl, p-terphenyl, naphthyl, anthryl, phenanthryl, pyrenyl, indenyl, fluorenyl, tetrahydronaphthyl, perylenyl, chrysenyl, naphthacenyl, and fluoranthenyl. At least one hydrogen atom of the aryl may be substituted by a deuterium atom, a halogen atom, a hydroxy, a nitro, a cyano, a silyl, an amino (—NH₂, —NH(R), —N(R′) (R″) wherein R′ and R″ are each independently an alkyl of 1 to 10 carbon atoms, in this case, called “alkylamino”), an amidino, a hydrazine, a hydrazone, a carboxyl, a sulfonic acid, a phosphoric acid, an alkyl of 1 to 24 carbon atoms, a halogenated alkyl of 1 to 24 carbon atoms, an alkenyl of 2 to 24 carbon atoms, an alkynyl of 2 to 24 carbon atoms, a heteroalkyl of 1 to 24 carbon atoms, an aryl of 6 to 24 carbon atoms, an arylalkyl of 7 to 24 carbon atoms, an alkylaryl of 7 to 24 carbon atoms, a heteroaryl of 2 to 24 carbon atoms, a heteroarylalkyl of 3 to 24 carbon atoms, or an alkylheteroaryl of 3 to 24 carbon atoms.

As used herein, the term “aromatic hydrocarbon ring” refers to an aromatic ring composed of carbon and hydrogen atoms and the term “aliphatic hydrocarbon ring” refers to a hydrocarbon ring that is composed of carbon and hydrogen atoms, but does not belong to the aromatic hydrocarbon rings. Particularly, the aliphatic hydrocarbon ring may have a bonding structure of the sp³ orbital for at least 30% of the carbon atoms as ring members, with 0 to 3 double and/or triple bonds within the ring. More particularly, the aliphatic hydrocarbon ring may have a bonding structure of the sp³ orbital for at least 50% of the carbon atoms as ring members, with 0 to 2 double and/or triple bonds within the ring.

The term “aliphatic hydrocarbon ring-fused aryl”, as used herein, refers to a cyclic radical in which two adjacent carbon atoms as ring members of an aliphatic hydrocarbon ring and two adjacent carbon atoms as ring members of an aryl are fused with each other to share one double bond therebetween, with non-aromaticity across the molecule. Concrete examples include, but are not limited to, tetrahydronaphthyl, tetrahydrobenzocycloheptene, tetrahydrophenanthrene, tetrahydroanthracenyl, and octahydrotriphenylene.

The substituent “heteroaryl”, used in the compound of the present disclosure, means a hetero aromatic radical of 2 to 24 carbon atoms, bearing as ring member(s) one to three heteroatoms selected from among N, O, P, Si, S, Ge, Se, and Te. In the aromatic radical, two or more rings may be fused. One or more hydrogen atoms on the heteroaryl may be substituted by the same substituents as on the aryl.

In addition, the term “heteroaromatic ring”, as used herein, refers to an aromatic hydrocarbon ring bearing at least one heteroatom as aromatic ring member. In the heteroaromatic ring, one to three carbon atoms of the aromatic hydrocarbon may be substituted by at least one selected particularly from N, O, P, Si, S, Ge, Se, and Te.

The term “aliphatic hydrocarbon ring-fused heteroaryl” is same as “aliphatic hydrocarbon ring-fused aryl”, with the exception that the aryl is substituted by a heteroaryl. Concrete examples include tetrahydroindol, tetrahydrobenzofuranyl, tetrahydrobenzothiophene, tetrahydrocarbazole, tetrahydrodibenzofuranyl, tetrahydrodibenzothiophene, tetrahydroquinoline and tetrahydroquinoxaline.

In addition, the term “heteroaromatic ring”, as used herein, refers to an aromatic hydrocarbon ring bearing at least one heteroatom as an aromatic ring member. In the heteroaromatic ring, one to three carbon atoms of the aromatic hydrocarbon may be substituted by at least one selected particularly from N, O, P, Si, S, Ge, Se, and Te.

As used herein, the term “fused ring in which an aromatic hydrocarbon ring is fused with an aliphatic hydrocarbon ring” means a fused ring in which an aromatic hydrocarbon ring has two adjacent carbon atoms in common with an aliphatic hydrocarbon ring, as exemplified by a tetrahydronaphthalene ring in which the benzene ring shares two adjacent carbon atoms with the cyclohexane ring.

In the present disclosure, the term “fused ring having a heteroaromatic ring and an aliphatic hydrocarbon ring fused to each other” refer to a fused ring in which two adjacent carbon atoms as ring members of a heteroaromatic hydrocarbon ring and two adjacent carbon atoms as ring members of an aliphatic hydrocarbon ring are fused with each other to share two carbon atoms therebetween, as exemplified by hexahydrodibenzofuran ring in which a benzofuran ring and a cyclohexane ring are fused by sharing their respective two adjacent carbon atoms as ring members.

As used herein, the term “alkyl” refers to an alkane missing one hydrogen atom and includes linear or branched structures. Examples of the alkyl substituent useful in the present disclosure include methyl, ethyl, propyl, isopropyl, isobutyl, sec-butyl, tert-butyl, pentyl, iso-amyl, hexyl, and the like. At least one hydrogen atom of the alkyl may be substituted by the same substituent as in the aryl.

The term “cyclo” as used in substituents of the compounds of the present disclosure, such as cycloalkyl, cycloalkoxy, etc., refers to a structure responsible for a mono- or polycyclic ring of saturated hydrocarbons within an alkyl radical, an alkoxy radical, etc. Concrete examples of cycloalkyl radicals include cyclopropyl, cyclobutyl, cyclopentyl, cyclohexyl, methylcyclopentyl, methylcyclohexyl, ethylcyclopentyl, ethylcyclohexyl, adamantyl, dicyclopentadienyl, decahydronaphthyl, norbornyl, bornyl, isobornyl, and so on. One or more hydrogen atoms on the cycloalkyl may be substituted by the same substituents as on the aryl. This is true of the cycloalkoxy.

In the present disclosure, the term “heterocycloalkyl” refers to a cycloalkyl with at least one heteroatom substituted as a ring member for a carbon atom within the ring. Preferably, one to three carbon atoms within the ring may be substituted by at least one selected from N, O, P, S, Si, Ge, Se, and Te.

The aromatic hydrocarbon ring- or heteroaromatic ring-fused cycloalkyl refers to a cyclic radical in which two adjacent carbon atoms as ring members of an aromatic or heteroaromatic hydrocarbon ring and two adjacent carbon atoms as ring members of a cycloalkyl are fused with each other to share one double bond therebetween, with non-aromaticity across the molecule. Concrete examples include tetrahydronaphthyl, tetrahydrophenanthrene, tetrahydroquinoline, tetrahydroquinoxaline, and cyclopentabenzofurane.

The term “aromatic hydrocarbon ring-heterocyclic alkyl” is same as the aromatic hydrocarbon ring-fused cycloalkyl, with the exception that at least one carbon atom within the cycloalkyl ring is substituted by a heteroatom, with non-aromaticity across the molecule. Preferably, one to three carbon atoms within the cycloalkyl ring are substituted by at least one selected from N, O, P, S, Si, Ge, Se, and Te. Concrete examples of aromatic hydrocarbon ring-heterocyclic alkyl include hexahydrodibenzofuranyl, hexahydrocarbozole, hexahydrodibenzothiophene, and dihydrobenzodioxine.

As used herein, the “heteroaliphatic ring-fused aryl or heteroaryl” is same as the aliphatic hydrocarbon ring-fused aryl or heteroaryl, except for a heteroaliphatic ring substituted for the aliphatic hydrocarbon ring, with non-aromaticity across the molecule thereof. Concrete examples include cromane, dihydropyranopyridine, thiocromane, dihydrobenzodioxine, dihydrothiopyranopyridine, and dihydropyranopyrimidine.

The term “heteroaliphatic ring” refers to an aliphatic hydrocarbon bearing at least one heteroatom as a ring member. Preferably, one to three carbon atoms within an aliphatic hydrocarbon are substituted by at least one heteroatom selected from N, O, and S.

The term “alkoxy” as used in the compounds of the present disclosure refers to an alkyl or cycloalkyl singularly bonded to oxygen. Concrete examples of the alkoxy include methoxy, ethoxy, propoxy, isobutoxy, sec-butoxy, pentoxy, iso-amyloxy, hexyloxy, cyclobutyloxy, cyclopentyloxy, adamantyloxy, dicyclopentyloxy, bornyloxy, isobornyloxy, and the like. One or more hydrogen atoms on the alkoxy may be substituted by the same substituents as on the aryl.

Concrete examples of the arylalkyl used in the compounds of the present disclosure include phenylmethyl (benzyl), phenylethyl, phenylpropyl, naphthylmethyl, naphthylethyl, and the like. One or more hydrogen atoms on the arylalkyl may be substituted by the same substituents as on the aryl.

Concrete examples of the alkylaryl used in the compound of the present disclosure include tolyl, xylenyl, dimethylnaphthyl, t-butylphenyl, t-butylnaphthyl, and t-butylphenanthryl. One or more hydrogen atoms on the alkylaryl may be substituted by the same substituents as on the aryl.

Concrete examples of the silyl used in the compound of the present disclosure include trimethylsilyl, triethylsilyl, triphenylsilyl, trimethoxysilyl, dimethoxyphenylsilyl, diphenylmethylsilyl, diphenylvinylsilyl, methylcyclobutylsilyl, and dimethylfurylsilyl. One or more hydrogen atoms on the silyl may be substituted by the same substituents as on the aryl.

As used herein, the term “alkenyl” refers to an alkyl substituent containing a carbon-carbon double bond between two carbon atoms and the term “alkynyl” refers to an alkyl substituent containing a carbon-carbon triple bond between two carbon atoms.

As used herein, the term “alkylene” refers to an organic radical regarded as derived from an alkane by removal two hydrogen atoms from one carbon atom for methylene or different carbon atoms for ethylene or higher, such as methylene, ethylene, propylene, isopropylene, isobutylene, sec-butylene, tert-butylene, pentylene, iso-amylene, hexylene, and the like. One or more hydrogen atoms on the alkylene may be substituted by the same substituents as on the aryl.

In the present disclosure, the term “diarylamino” refers to an amine with two identical or different aryl radicals bonded to the nitrogen atom thereof. Furthermore, the term “diheteroarylamino”, as used herein, refers to an amine with two identical or different diheteroaryl radicals bonded to the nitrogen atom thereof. The term “aryl(heteroaryl)amino” means an amine with an aryl radical and a heteroaryl radical both bonded to the nitrogen atom thereof.

Also, a preferable example of the substituted or unsubstituted aromatic hydrocarbon ring-fused cycloalkyl of 7 to 30 carbon atoms in the present disclosure may be a substituted or unsubstituted aromatic hydrocarbon ring-fused cycloalkyl of 9 to 20 carbon atoms.

In a preferred embodiment of the present disclosure, the substituted or unsubstituted heteroaromatic ring-fused cycloalkyl of 5 to 30 carbon atoms may be a substituted or unsubstituted heteroaromatic ring-fused cycloalkyl of 7 to 20 carbon atoms.

In a preferred embodiment of the present disclosure, the substituted or unsubstituted aromatic hydrocarbon ring-fused heterocycloalkyl of 6 to 30 carbon atoms may be a substituted or unsubstituted aromatic hydrocarbon ring-fused heterocycloalkyl of 9 to 20 carbon atoms.

In a preferred embodiment of the present disclosure, the substituted or unsubstituted aliphatic hydrocarbon ring-fused aryl of 7 to 30 carbon atoms may be a substituted or unsubstituted aliphatic hydrocarbon ring-fused aryl of 8 to 20 carbon atoms.

In a preferred embodiment of the present disclosure, the substituted or unsubstituted aliphatic hydrocarbon ring-fused heteroaryl of 5 to 30 carbon atoms may be a substituted or unsubstituted aliphatic hydrocarbon ring-fused heteroaryl of 7 to 20 carbon atoms.

In a preferred embodiment of the present disclosure, the substituted or unsubstituted heteroaliphatic ring-fused aryl of 6 to 30 carbon atoms may be a substituted or unsubstituted heteroaliphatic ring-fused aryl of 7 to 20 carbon atoms.

In a preferred embodiment of the present disclosure, the substituted or unsubstituted heteroaliphatic ring-fused heteroaryl of 5 to 30 carbon atoms may be a substituted or unsubstituted heteroaliphatic ring-fused heteroaryl of 6 to 20 carbon atoms.

Moreover, the expression “linkage may be made between R₁₂ and R₁₃, between R₁₄ and R₁₅, and/or R₁₆ and R₁₇ to additionally form an aliphatic or aromatic mono- or polycyclic ring” means that R₁₂ and R₁₃ may each be cleared of one hydrogen radical and connected to each other to additionally form a ring, R₁₄ and R₁₅ may each be cleared of one hydrogen radical and connected to each other to additionally form a ring, and/or R₁₆ and R₁₇ may each be cleared of one hydrogen radical and connected to each other to additionally form a ring.

Moreover, the expression “at least one of R₁₀ to R₁₇ may be connected to at least one of A₁ to A₃ rings to additionally form an aliphatic or aromatic mono- or polycyclic ring” means that a hydrogen atom is removed from at least one of A₁ to A₃ rings and at least one of R₁₀ to R₁₇ and the resulting radicals, each missing one hydrogen, are boned to each other to additionally form a ring. In addition, the expression “when at least two of Z₁ to Z₁₀ are each CR₃₁, the corresponding R₃₁'s are connected to each other to additionally form an aliphatic or aromatic mono- or polycyclic ring” can also be interpreted the same way. The meaning is true of all the expressions “ . . . connected to additionally form a ring”, which are to be given in the description and claims.

In addition, preferred examples of the substituent for the term “substituted” in the expression “substituted or unsubstituted” used for compounds of Chemical Formulas A, 3, 4, and 3-1 to 4-3 include a deuterium atom, a cyano, a halogen, a hydroxy, a nitro, an alkyl of 1 to 12 carbon atoms, a halogenated alkyl of 1 to 12 carbon atoms, an alkenyl of 2 to 12 carbon atoms, an alkynyl of 2 to 12 carbon atoms, a cycloalkyl of 3 to 12 carbon atoms, a heteroalkyl of 1 to 12 carbon atoms, an aryl of 6 to 18 carbon atoms, an arylakyl of 7 to 20 carbon atoms, an alkylaryl of 7 to 20 carbon atoms, a heteroaryl of 2 to 18 carbon atoms, a heteroarylalkyl of 3 to 18 carbon atoms, an alkylheteroaryl of 3 to 18 carbon atoms, an aromatic hydrocarbon ring-fused cycloalkyl of 9 to 20 carbon atoms, a heteroaromatic ring-fused cycloalkyl of 7 to 20 carbon atoms, an aromatic hydrocarbon ring-fused heterocycloalkyl of 9 to 20 carbon atoms, an aliphatic hydrocarbon ring-fused aryl of 8 to 20 carbon atoms, an aliphatic hydrocarbon ring-fused heteroaryl of 7 to 20 carbon atoms, an alkoxy of 1 to 12 carbon atoms, an amine of 1 to 30 carbon atoms, a silyl of 1 to 30 carbon atoms, a germanium of 1 to 30 carbon atoms, an aryloxy of 6 to 18 carbon atoms, and an arylthionyl of 6 to 18 carbon atoms, with at least one hydrogen atom of the substituent being substitutable with a deuterium atom.

In the present disclosure, the anthracene compound represented by Chemical Formula A is characterized by the structure in which a phenyl radical is directly bonded to the carbon atom at position 9 in an anthracene moiety and has two phenyl substituents at the ortho positions, the phenyl substituents containing R and R′, respectively, with the n number (0 to 60) of deuterium atoms (Do), instead of hydrogen atoms, being present across the anthracene compound represented by Chemical Formula.

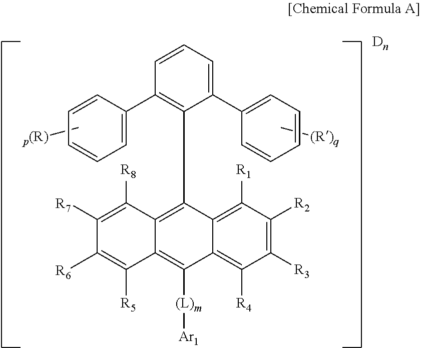

In an embodiment of the present disclosure, the anthracene compound represented by Chemical Formula A may be an anthracene compound represented by any one of the following Chemical Formulas A-1 to A-4:

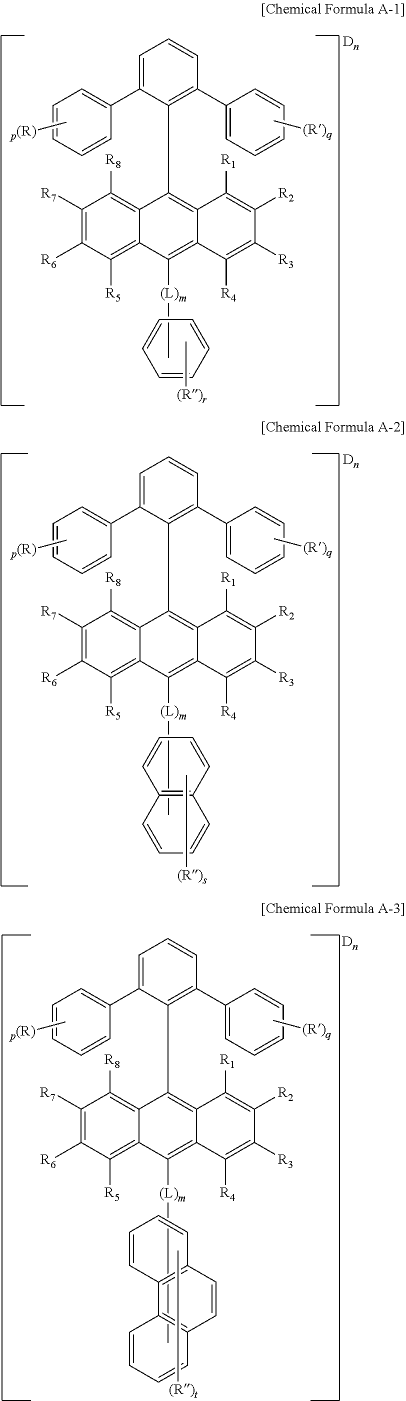

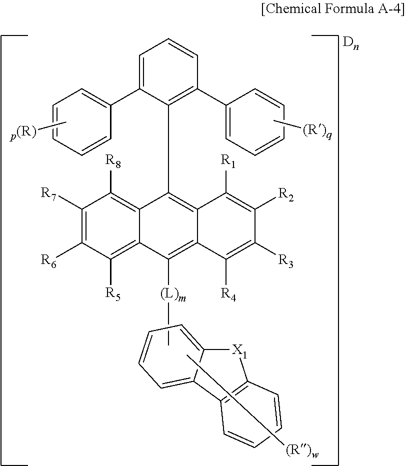

wherein
R, R′, R₁ to R₈, L, m, Dₙ, p, and q are as defined in Chemical Formula A,
R″ is any one selected from a hydrogen atom, a deuterium atom, a substituted or unsubstituted alkyl of 1 to 30 carbon atoms, a substituted or unsubstituted alkenyl of 2 to 30 carbon atoms, a substituted or unsubstituted aryl of 6 to 50 carbon atoms, a substituted or unsubstituted cycloalkyl of 3 to 30 carbon atoms, a substituted or unsubstituted heterocycloalkyl of 3 to 30 carbon atoms, a substituted or unsubstituted heteroaryl of 2 to 50 carbon atoms, a substituted or unsubstituted aromatic hydrocarbon ring-fused cycloalkyl of 7 to 30 carbon atoms, a substituted or unsubstituted heteroaromatic ring-fused cycloalkyl of 5 to 30 carbon atoms, a substituted or unsubstituted aromatic hydrocarbon ring-fused heterocycloalkyl of 6 to 30 carbon atoms, a substituted or unsubstituted aliphatic hydrocarbon ring-fused aryl of 7 to 30 carbon atoms, a substituted or unsubstituted aliphatic hydrocarbon ring-fused heteroaryl of 5 to 30 carbon atoms, a substituted or unsubstituted heteroaliphatic ring-fused aryl of 6 to 30 carbon atoms, a substituted or unsubstituted heteroaliphatic ring-fused heteroaryl of 5 to 30 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted amine of 0 to 30 carbon atoms, a substituted or unsubstituted silyl of 0 to 30 carbon atoms, a substituted or unsubstituted germanium of 0 to 30 carbon atoms, a nitro, a cyano, and a halogen,
r is an integer of 1 to 5, wherein when r is 2 or higher, the corresponding R″ radicals are same or different,
s is an integer of 1 to 7, wherein when s is 2 or higher, the corresponding R″ radicals are same or different,
t is an integer of 1 to 9, wherein when t is 2 or higher, the corresponding R″ radicals are same or different,
w is an integer of 1 to 7, wherein when w is 2 or higher, the corresponding R″ radicals are same or different,
in Chemical Formula A-4,
X₁ is a linker selected from O, S, CR₁₈R₁₉, and NR₂₀,
R₁₈ to R₂₀, which are same or different, are each independently any one selected from a substituted or unsubstituted alkyl of 1 to 30 carbon atoms and a substituted or unsubstituted aryl of 6 to 50 carbon atoms, and
R₁₈ and R₁₉ may be connected to each other to additionally form an aliphatic or aromatic mono- or polycyclic ring.

In an embodiment of the present disclosure, R and R′ in Chemical Formula A, which are same or different, may be a hydrogen atom or a deuterium atom.

In an embodiment of the present disclosure, L in Chemical Formula A may be a single bond or any one selected from the following Structural Formulas 1 to 5:

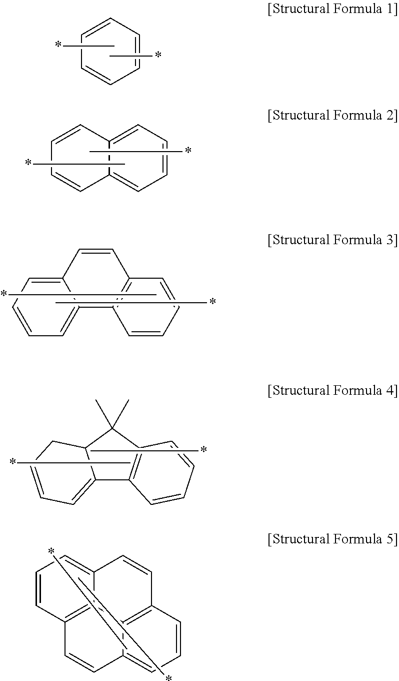

A hydrogen atom or a deuterium atom may be bonded to an aromatic carbon atom of Structural Formulas 1 to 5.

In an embodiment of the present disclosure, R, R′, and R₁ to Re, which are same or different, are each independently any one selected from a hydrogen atom, a deuterium atom, a substituted or unsubstituted alkyl of 1 to 15 carbon atoms, a substituted or unsubstituted aryl of 6 to 20 carbon atoms, a substituted or unsubstituted cycloalkyl of 3 to 15 carbon atoms, a substituted or unsubstituted heteroaryl of 2 to 20 carbon atoms, a cyano, and a halogen.

According to an embodiment of the present disclosure, n in Chemical Formula A may be an integer of 4 to 50, preferably an integer of 5 to 45, more preferably an integer of 6 to 40, much more an integer of 7 to 35, even much more an integer of 8 to 32, and most preferably at an integer of 9 to 30.

According to an embodiment of the present disclosure, at least four of R₁ to R₈ in Chemical Formula A may each be a deuterium atom and n is an integer of 4 to 50, preferably 5 to 45, more preferably 6 to 40, and even more preferably 7 to 35.

According to an embodiment of the present disclosure, Ar₁ in Chemical Formula A may be a substituted or unsubstituted aryl of 6 to 20 carbon atoms or a substituted or unsubstituted heteroaryl of 3 to 20 carbon atoms.

In an embodiment of the present disclosure, X₁ in Chemical Formula A-4 may be O or S.

In an embodiment of the present disclosure, R and R′ in Chemical Formulas A and B may be each a deuterium atom.

Furthermore, concrete examples of the anthracene compound represented by Chemical Formula A include, but are not limited to, the following [BH-1] to [BH-100]:

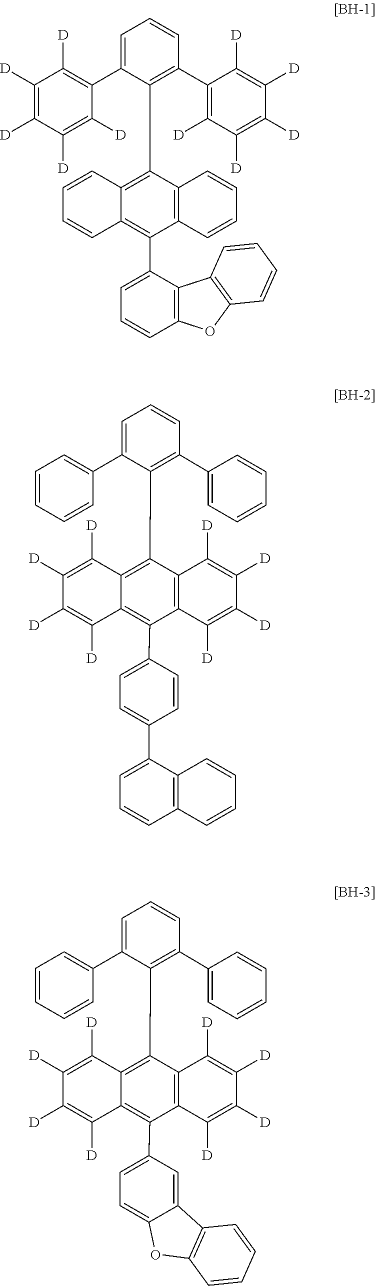 [SMILES: [2H]c1c([2H])c([2H])c(-c2cccc(-c3c([2H])c([2H])c([2H])c([2H])c3[2H])c2-c2c3ccccc3c(-c3cccc4oc5ccccc5c34)c3ccccc23)c([2H])c1[2H] ; [2H]c1c([2H])c([2H])c2c(-c3c(-c4ccccc4)cccc3-c3ccccc3)c3c([2H])c([2H])c([2H])c([2H])c3c(-c3ccc(-c4cccc5ccccc45)cc3)c2c1[2H] ; [2H]c1c([2H])c([2H])c2c(-c3c(-c4ccccc4)cccc3-c3ccccc3)c3c([2H])c([2H])c([2H])c([2H])c3c(-c3ccc4oc5ccccc5c4c3)c2c1[2H]]

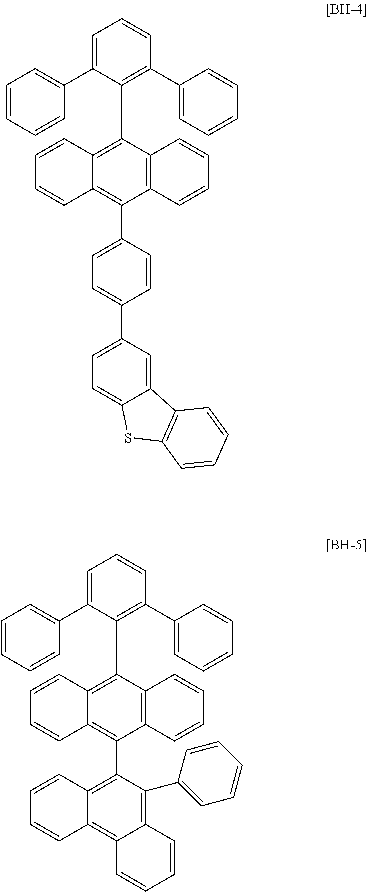

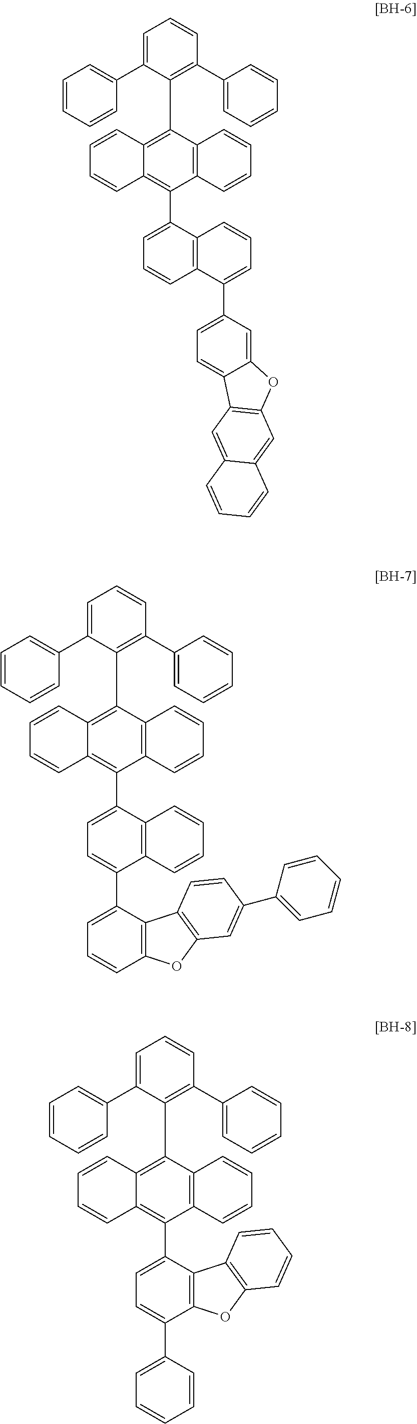 [SMILES: c1ccc(-c2cccc(-c3ccccc3)c2-c2c3ccccc3c(-c3cccc4c(-c5ccc6c(c5)oc5cc7ccccc7cc56)cccc34)c3ccccc23)cc1 ; c1ccc(-c2ccc3c(c2)oc2cccc(-c4ccc(-c5c6ccccc6c(-c6c(-c7ccccc7)cccc6-c6ccccc6)c6ccccc56)c5ccccc45)c23)cc1 ; c1ccc(-c2cccc(-c3ccccc3)c2-c2c3ccccc3c(-c3ccc(-c4ccccc4)c4oc5ccccc5c34)c3ccccc23)cc1]

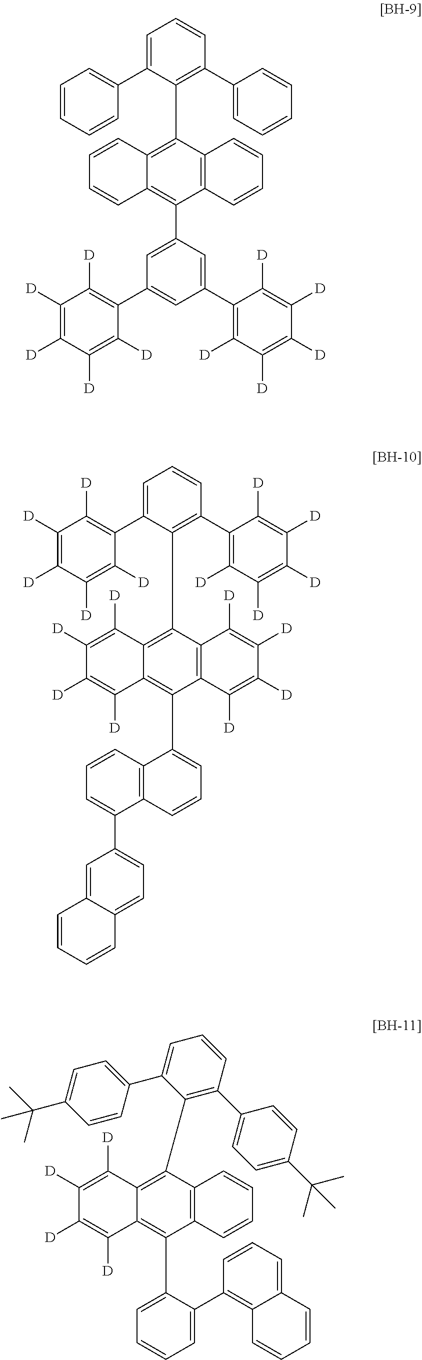 [SMILES: [2H]c1c([2H])c([2H])c(-c2cc(-c3c([2H])c([2H])c([2H])c([2H])c3[2H])cc(-c3c4ccccc4c(-c4c(-c5ccccc5)cccc4-c4ccccc4)c4ccccc34)c2)c([2H])c1[2H] ; [2H]c1c([2H])c([2H])c(-c2cccc(-c3c([2H])c([2H])c([2H])c([2H])c3[2H])c2-c2c3c([2H])c([2H])c([2H])c([2H])c3c(-c3cccc4c(-c5ccc6ccccc6c5)cccc34)c3c([2H])c([2H])c([2H])c([2H])c23)c([2H])c1[2H] ; [2H]c1c([2H])c([2H])c2c(-c3c(-c4ccc(C(C)(C)C)cc4)cccc3-c3ccc(C(C)(C)C)cc3)c3ccccc3c(-c3ccccc3-c3cccc4ccccc34)c2c1[2H]]

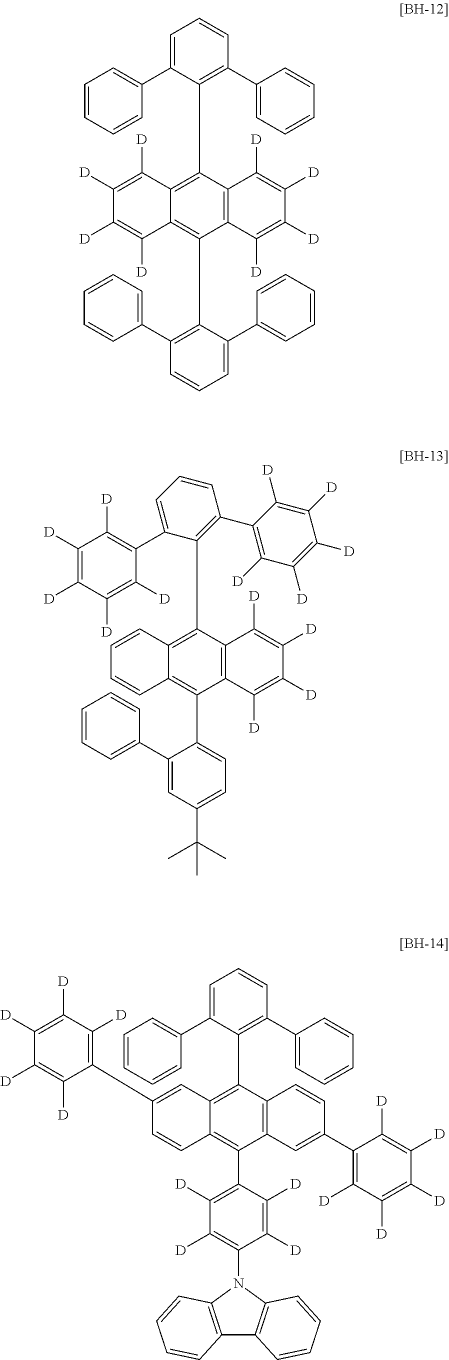 [SMILES: [2H]c1c([2H])c([2H])c2c(-c3c(-c4ccccc4)cccc3-c3ccccc3)c3c([2H])c([2H])c([2H])c([2H])c3c(-c3c(-c4ccccc4)cccc3-c3ccccc3)c2c1[2H] ; [2H]c1c([2H])c([2H])c(-c2cccc(-c3c([2H])c([2H])c([2H])c([2H])c3[2H])c2-c2c3ccccc3c(-c3ccc(C(C)(C)C)cc3-c3ccccc3)c3c([2H])c([2H])c([2H])c([2H])c23)c([2H])c1[2H] ; [2H]c1c([2H])c([2H])c(-c2ccc3c(-c4c([2H])c([2H])c(-n5c6ccccc6c6ccccc65)c([2H])c4[2H])c4cc(-c5c([2H])c([2H])c([2H])c([2H])c5[2H])ccc4c(-c4c(-c5ccccc5)cccc4-c4ccccc4)c3c2)c([2H])c1[2H]]

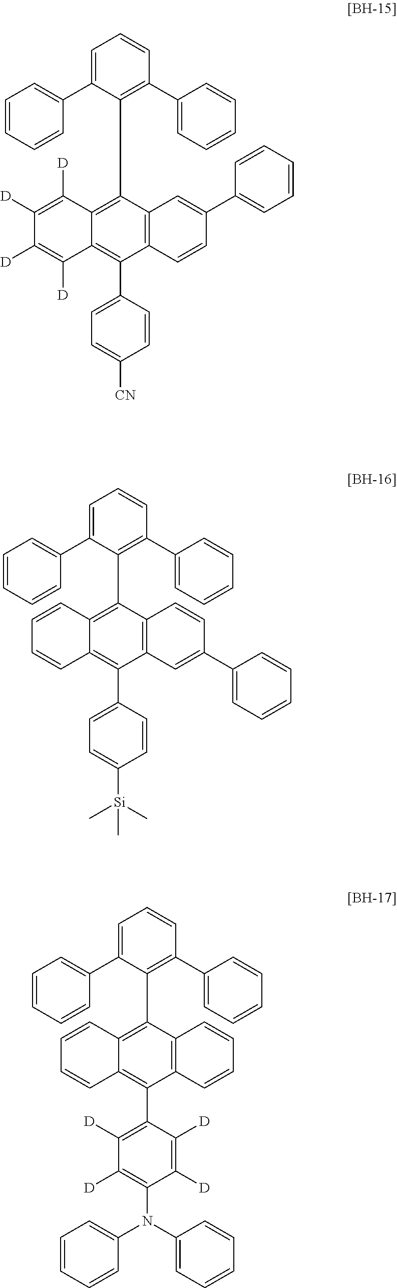 [SMILES: [2H]c1c([2H])c([2H])c2c(-c3c(-c4ccccc4)cccc3-c3ccccc3)c3cc(-c4ccccc4)ccc3c(-c3ccc(C#N)cc3)c2c1[2H] ; C[Si](C)(C)c1ccc(-c2c3ccccc3c(-c3c(-c4ccccc4)cccc3-c3ccccc3)c3ccc(-c4ccccc4)cc23)cc1 ; [2H]c1c([2H])c(N(c2ccccc2)c2ccccc2)c([2H])c([2H])c1-c1c2ccccc2c(-c2c(-c3ccccc3)cccc2-c2ccccc2)c2ccccc12]

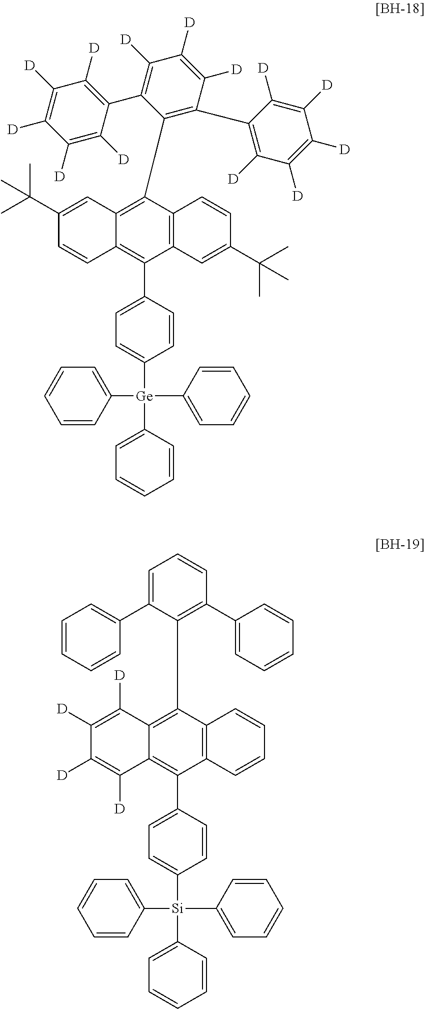

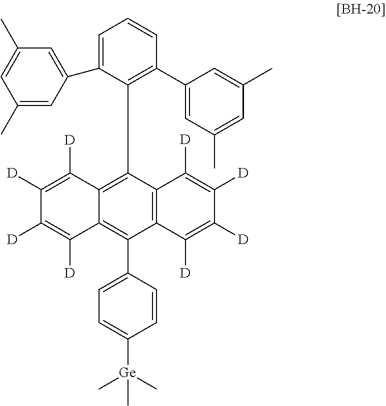

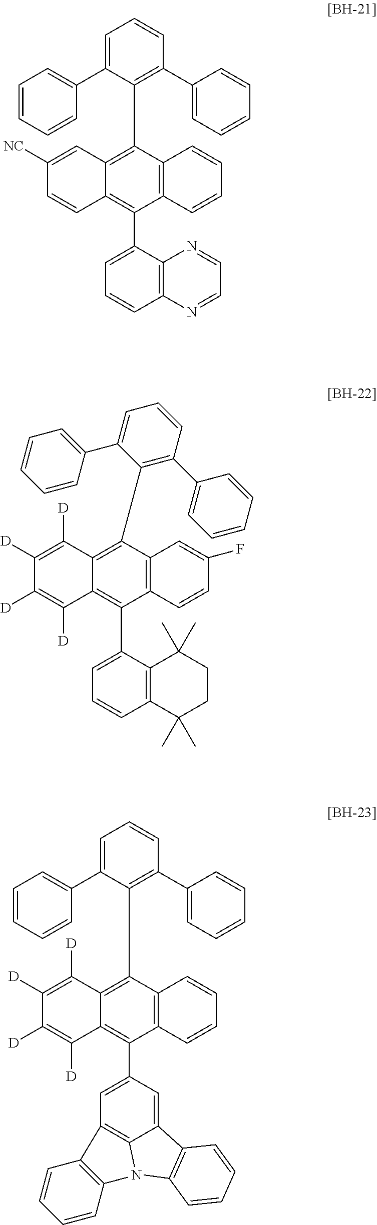 [SMILES: N#Cc1ccc2c(-c3cccc4nccnc34)c3ccccc3c(-c3c(-c4ccccc4)cccc3-c3ccccc3)c2c1 ; [2H]c1c([2H])c([2H])c2c(-c3c(-c4ccccc4)cccc3-c3ccccc3)c3cc(F)ccc3c(-c3cccc4c3C(C)(C)CCC4(C)C)c2c1[2H] ; [2H]c1c([2H])c([2H])c2c(-c3c(-c4ccccc4)cccc3-c3ccccc3)c3ccccc3c(-c3cc4c5ccccc5n5c6ccccc6c(c3)c45)c2c1[2H]]

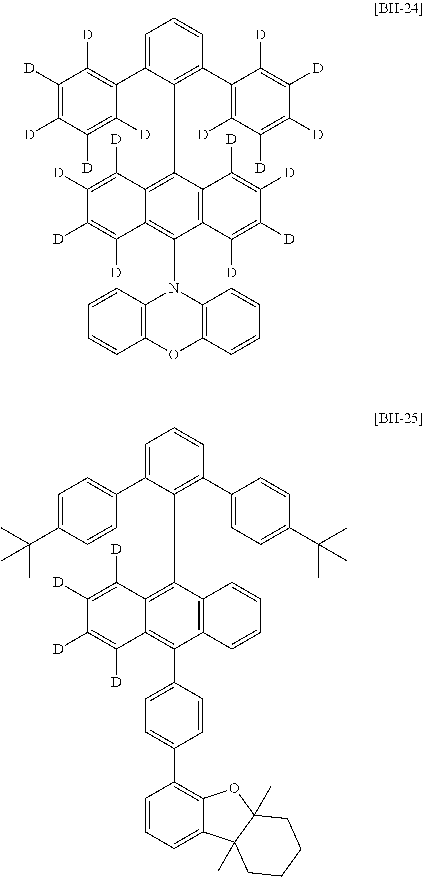 [SMILES: [2H]c1c([2H])c([2H])c(-c2cccc(-c3c([2H])c([2H])c([2H])c([2H])c3[2H])c2-c2c3c([2H])c([2H])c([2H])c([2H])c3c(N3c4ccccc4Oc4ccccc43)c3c([2H])c([2H])c([2H])c([2H])c23)c([2H])c1[2H] ; [2H]c1c([2H])c([2H])c2c(-c3c(-c4ccc(C(C)(C)C)cc4)cccc3-c3ccc(C(C)(C)C)cc3)c3ccccc3c(-c3ccc(-c4cccc5c4OC4(C)CCCCC54C)cc3)c2c1[2H]]

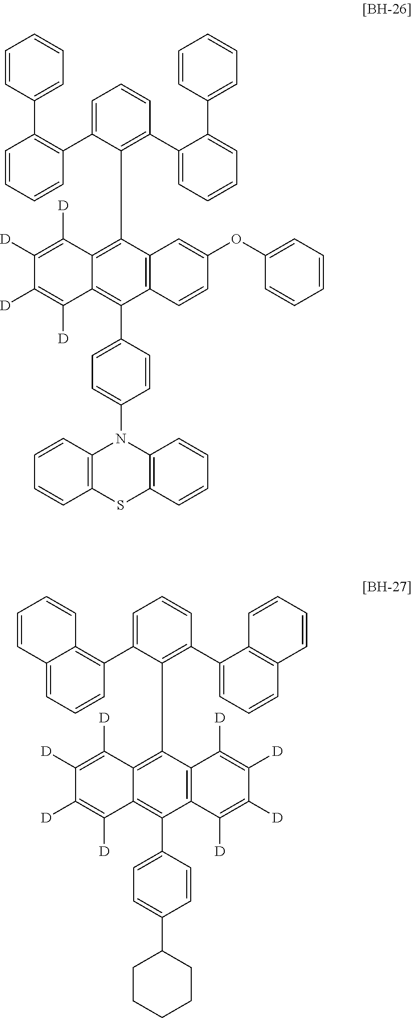 [SMILES: [2H]c1c([2H])c([2H])c2c(-c3c(-c4ccccc4-c4ccccc4)cccc3-c3ccccc3-c3ccccc3)c3cc(Oc4ccccc4)ccc3c(-c3ccc(N4c5ccccc5Sc5ccccc54)cc3)c2c1[2H] ; [2H]c1c([2H])c([2H])c2c(-c3c(-c4cccc5ccccc45)cccc3-c3cccc4ccccc34)c3c([2H])c([2H])c([2H])c([2H])c3c(-c3ccc(C4CCCCC4)cc3)c2c1[2H]]

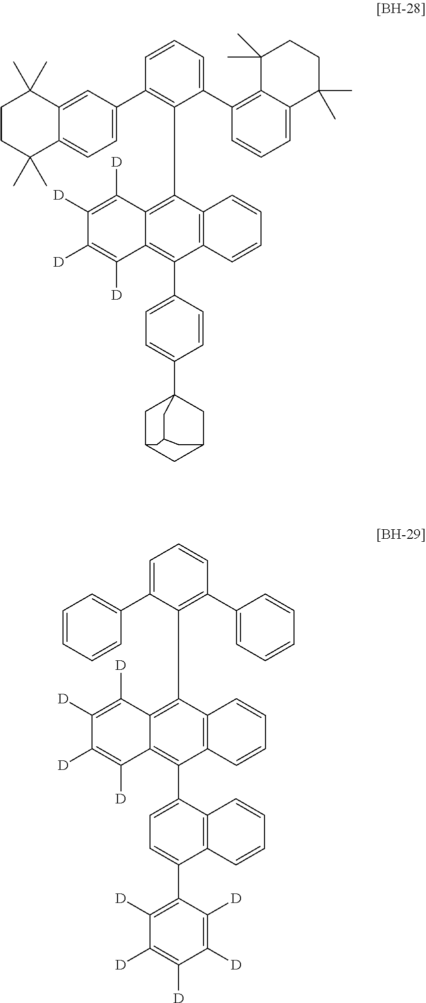 [SMILES: [2H]c1c([2H])c([2H])c2c(-c3c(-c4ccc5c(c4)C(C)(C)CCC5(C)C)cccc3-c3cccc4c3C(C)(C)CCC4(C)C)c3ccccc3c(-c3ccc(C45CC6CC(CC(C6)C4)C5)cc3)c2c1[2H] ; [2H]c1c([2H])c([2H])c(-c2ccc(-c3c4ccccc4c(-c4c(-c5ccccc5)cccc4-c4ccccc4)c4c([2H])c([2H])c([2H])c([2H])c34)c3ccccc23)c([2H])c1[2H]]

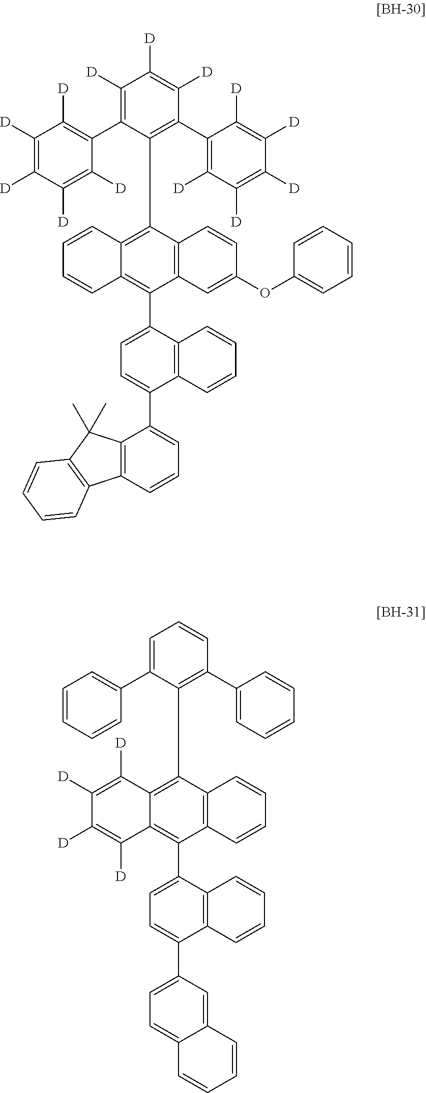 [SMILES: [2H]c1c([2H])c([2H])c(-c2c([2H])c([2H])c([2H])c(-c3c([2H])c([2H])c([2H])c([2H])c3[2H])c2-c2c3ccccc3c(-c3ccc(-c4cccc5c4C(C)(C)c4ccccc4-5)c4ccccc34)c3cc(Oc4ccccc4)ccc23)c([2H])c1[2H] ; [2H]c1c([2H])c([2H])c2c(-c3ccc(-c4ccc5ccccc5c4)c4ccccc34)c3ccccc3c(-c3c(-c4ccccc4)cccc3-c3ccccc3)c2c1[2H]]

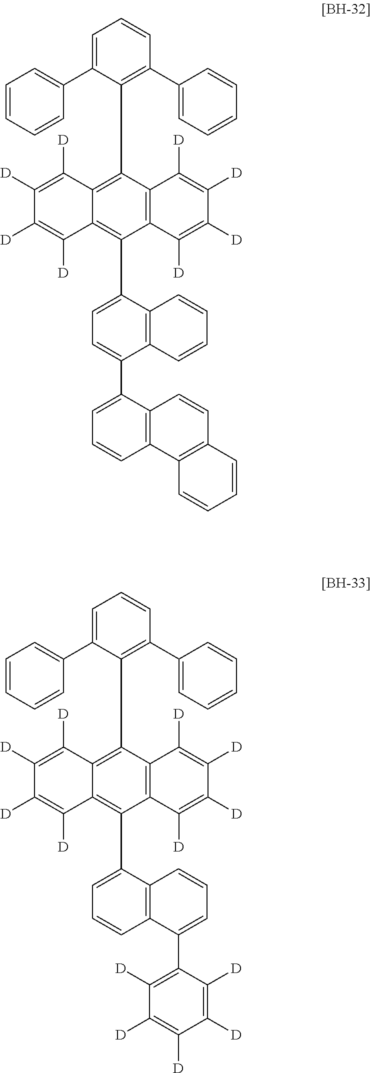 [SMILES: [2H]c1c([2H])c([2H])c2c(-c3ccc(-c4cccc5c4ccc4ccccc45)c4ccccc34)c3c([2H])c([2H])c([2H])c([2H])c3c(-c3c(-c4ccccc4)cccc3-c3ccccc3)c2c1[2H] ; [2H]c1c([2H])c([2H])c(-c2cccc3c(-c4c5c([2H])c([2H])c([2H])c([2H])c5c(-c5c(-c6ccccc6)cccc5-c5ccccc5)c5c([2H])c([2H])c([2H])c([2H])c45)cccc23)c([2H])c1[2H]]

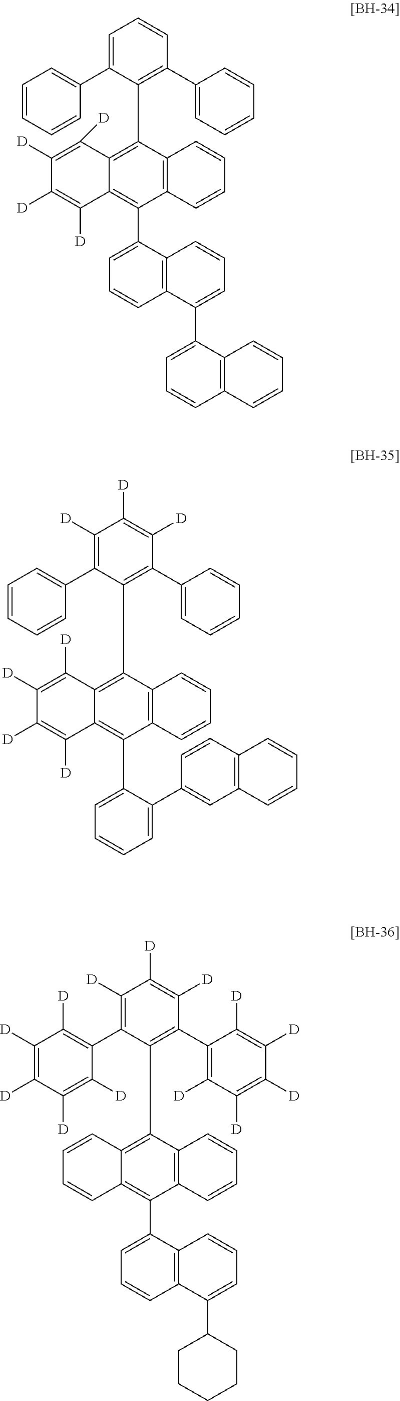 [SMILES: [2H]c1c([2H])c([2H])c2c(-c3cccc4c(-c5cccc6ccccc56)cccc34)c3ccccc3c(-c3c(-c4ccccc4)cccc3-c3ccccc3)c2c1[2H] ; [2H]c1c([2H])c(-c2ccccc2)c(-c2c3ccccc3c(-c3ccccc3-c3ccc4ccccc4c3)c3c([2H])c([2H])c([2H])c([2H])c23)c(-c2ccccc2)c1[2H] ; [2H]c1c([2H])c([2H])c(-c2c([2H])c([2H])c([2H])c(-c3c([2H])c([2H])c([2H])c([2H])c3[2H])c2-c2c3ccccc3c(-c3cccc4c(C5CCCCC5)cccc34)c3ccccc23)c([2H])c1[2H]]

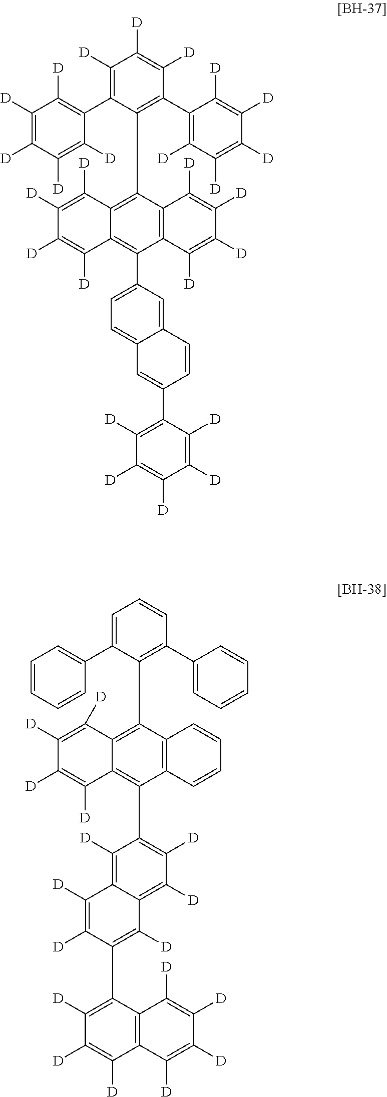 [SMILES: [2H]c1c([2H])c([2H])c(-c2ccc3cc(-c4c5c([2H])c([2H])c([2H])c([2H])c5c(-c5c(-c6c([2H])c([2H])c([2H])c([2H])c6[2H])c([2H])c([2H])c([2H])c5-c5c([2H])c([2H])c([2H])c([2H])c5[2H])c5c([2H])c([2H])c([2H])c([2H])c45)ccc3c2)c([2H])c1[2H] ; [2H]c1c(-c2c3ccccc3c(-c3c(-c4ccccc4)cccc3-c3ccccc3)c3c([2H])c([2H])c([2H])c([2H])c23)c([2H])c2c([2H])c([2H])c(-c3c([2H])c([2H])c([2H])c4c([2H])c([2H])c([2H])c([2H])c34)c([2H])c2c1[2H]]

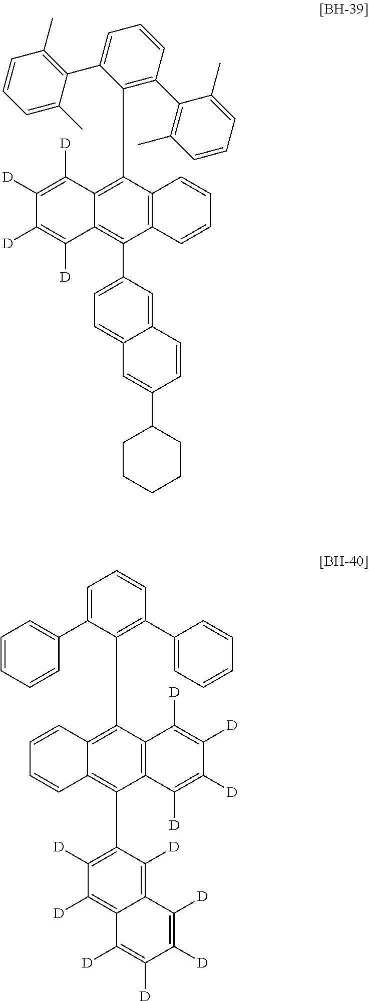 [SMILES: [2H]c1c([2H])c([2H])c2c(-c3c(-c4c(C)cccc4C)cccc3-c3c(C)cccc3C)c3ccccc3c(-c3ccc4cc(C5CCCCC5)ccc4c3)c2c1[2H] ; [2H]c1c([2H])c([2H])c2c([2H])c(-c3c4ccccc4c(-c4c(-c5ccccc5)cccc4-c4ccccc4)c4c([2H])c([2H])c([2H])c([2H])c34)c([2H])c([2H])c2c1[2H]]

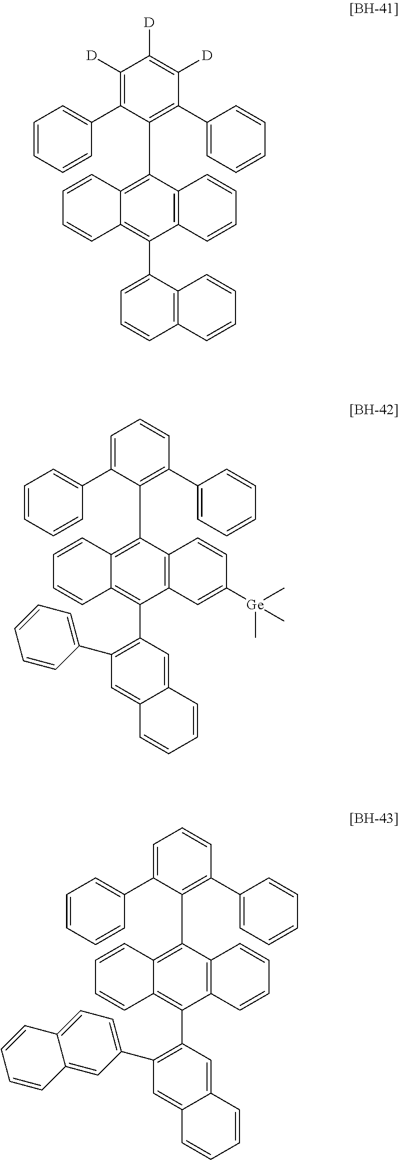

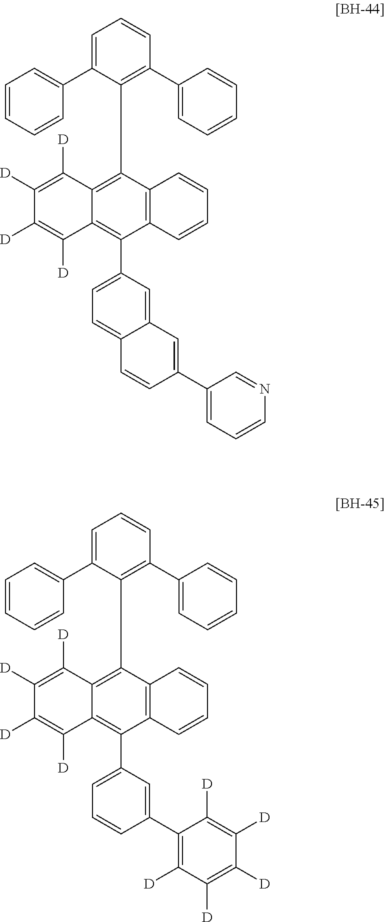 [SMILES: [2H]c1c([2H])c([2H])c2c(-c3c(-c4ccccc4)cccc3-c3ccccc3)c3ccccc3c(-c3ccc4ccc(-c5cccnc5)cc4c3)c2c1[2H] ; [2H]c1c([2H])c([2H])c(-c2cccc(-c3c4ccccc4c(-c4c(-c5ccccc5)cccc4-c4ccccc4)c4c([2H])c([2H])c([2H])c([2H])c34)c2)c([2H])c1[2H]]

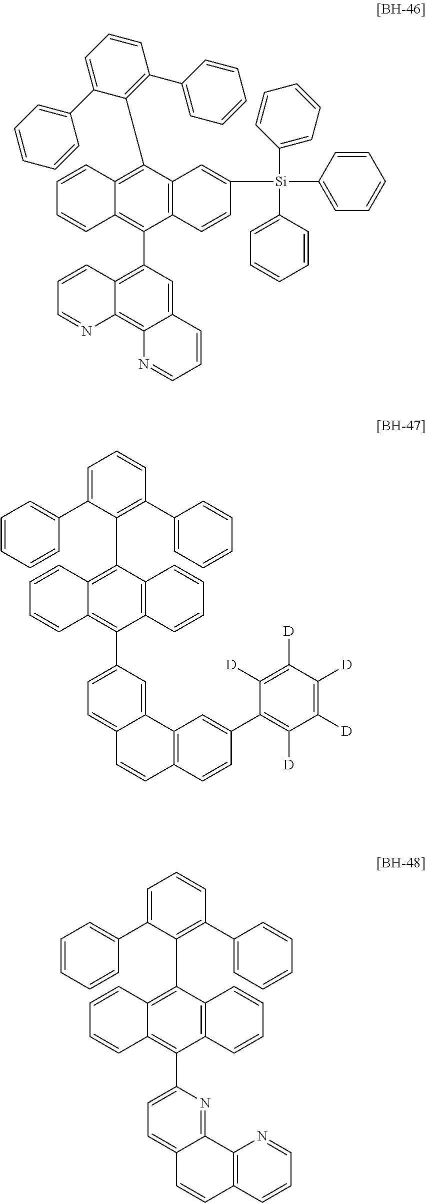 [SMILES: c1ccc(-c2cccc(-c3ccccc3)c2-c2c3ccccc3c(-c3cc4cccnc4c4ncccc34)c3ccc([Si](c4ccccc4)(c4ccccc4)c4ccccc4)cc23)cc1 ; [2H]c1c([2H])c([2H])c(-c2ccc3ccc4ccc(-c5c6ccccc6c(-c6c(-c7ccccc7)cccc6-c6ccccc6)c6ccccc56)cc4c3c2)c([2H])c1[2H] ; c1ccc(-c2cccc(-c3ccccc3)c2-c2c3ccccc3c(-c3ccc4ccc5cccnc5c4n3)c3ccccc23)cc1]

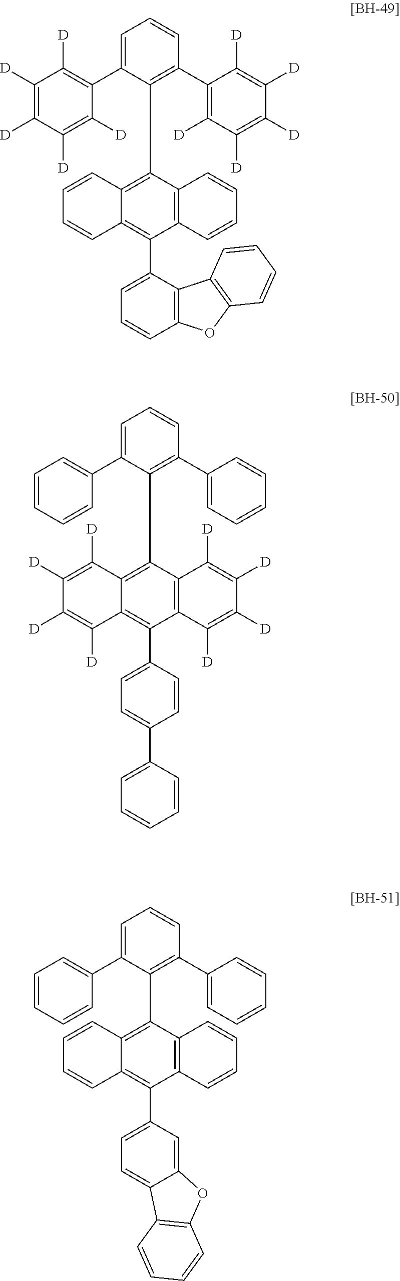 [SMILES: [2H]c1c([2H])c([2H])c(-c2cccc(-c3c([2H])c([2H])c([2H])c([2H])c3[2H])c2-c2c3ccccc3c(-c3cccc4oc5ccccc5c34)c3ccccc23)c([2H])c1[2H] ; [2H]c1c([2H])c([2H])c2c(-c3c(-c4ccccc4)cccc3-c3ccccc3)c3c([2H])c([2H])c([2H])c([2H])c3c(-c3ccc(-c4ccccc4)cc3)c2c1[2H] ; c1ccc(-c2cccc(-c3ccccc3)c2-c2c3ccccc3c(-c3ccc4c(c3)oc3ccccc34)c3ccccc23)cc1]

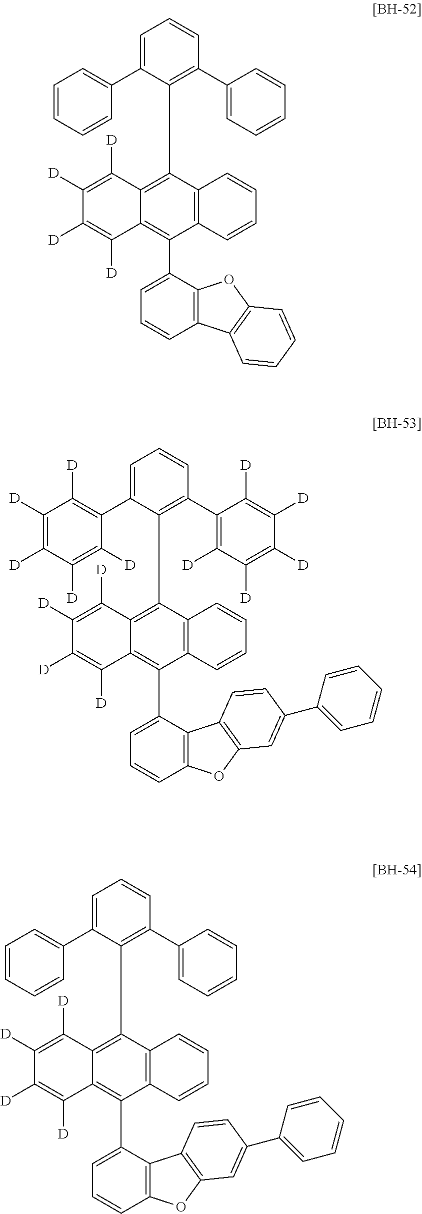 [SMILES: [2H]c1c([2H])c([2H])c2c(-c3cccc4c3oc3ccccc34)c3ccccc3c(-c3c(-c4ccccc4)cccc3-c3ccccc3)c2c1[2H] ; [2H]c1c([2H])c([2H])c(-c2cccc(-c3c([2H])c([2H])c([2H])c([2H])c3[2H])c2-c2c3ccccc3c(-c3cccc4oc5cc(-c6ccccc6)ccc5c34)c3c([2H])c([2H])c([2H])c([2H])c23)c([2H])c1[2H] ; [2H]c1c([2H])c([2H])c2c(-c3cccc4oc5cc(-c6ccccc6)ccc5c34)c3ccccc3c(-c3c(-c4ccccc4)cccc3-c3ccccc3)c2c1[2H]]

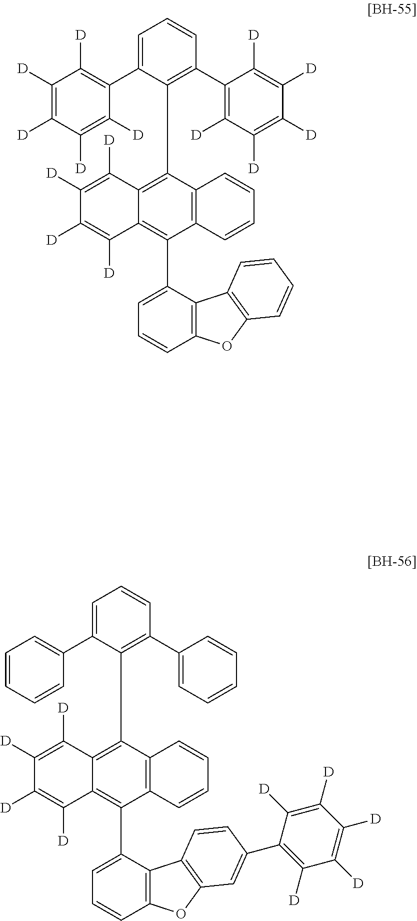 [SMILES: [2H]c1c([2H])c([2H])c(-c2cccc(-c3c([2H])c([2H])c([2H])c([2H])c3[2H])c2-c2c3ccccc3c(-c3cccc4oc5ccccc5c34)c3c([2H])c([2H])c([2H])c([2H])c23)c([2H])c1[2H] ; [2H]c1c([2H])c([2H])c(-c2ccc3c(c2)oc2cccc(-c4c5ccccc5c(-c5c(-c6ccccc6)cccc5-c5ccccc5)c5c([2H])c([2H])c([2H])c([2H])c45)c23)c([2H])c1[2H]]

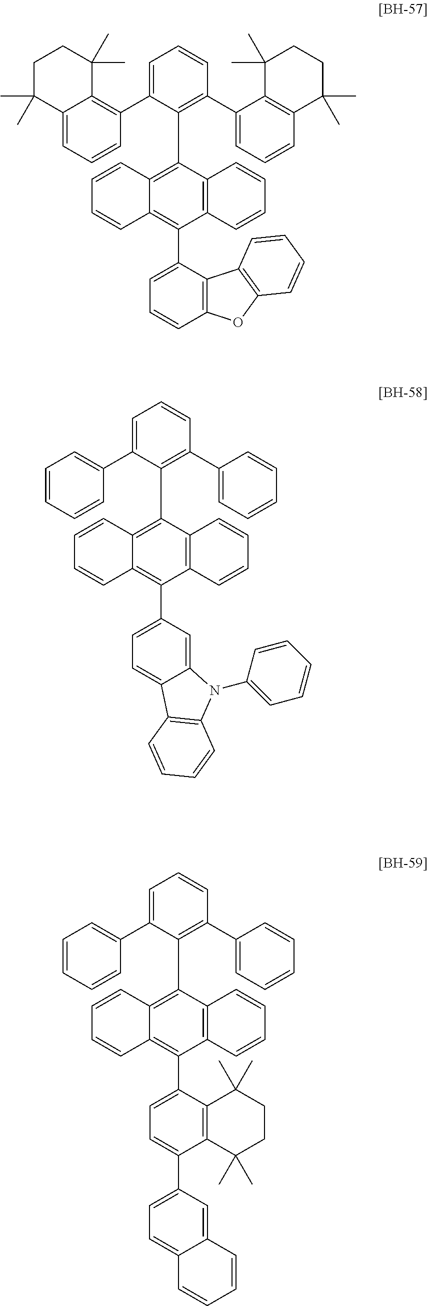

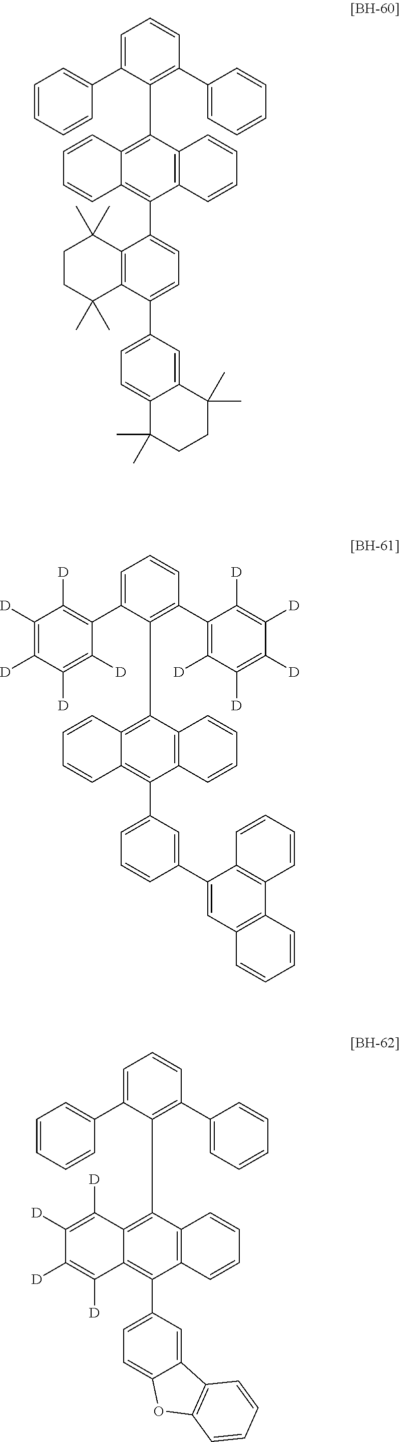

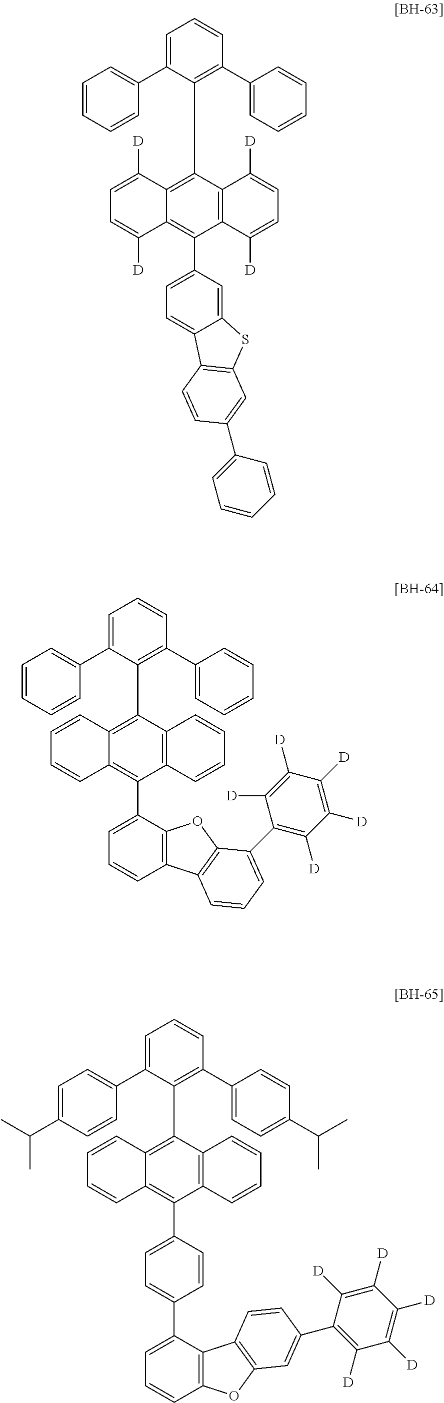 [SMILES: [2H]c1ccc([2H])c2c(-c3c(-c4ccccc4)cccc3-c3ccccc3)c3c([2H])ccc([2H])c3c(-c3ccc4c(c3)sc3cc(-c5ccccc5)ccc34)c12 ; [2H]c1c([2H])c([2H])c(-c2cccc3c2oc2c(-c4c5ccccc5c(-c5c(-c6ccccc6)cccc5-c5ccccc5)c5ccccc45)cccc23)c([2H])c1[2H] ; [2H]c1c([2H])c([2H])c(-c2ccc3c(c2)oc2cccc(-c4ccc(-c5c6ccccc6c(-c6c(-c7ccc(C(C)C)cc7)cccc6-c6ccc(C(C)C)cc6)c6ccccc56)cc4)c23)c([2H])c1[2H]]

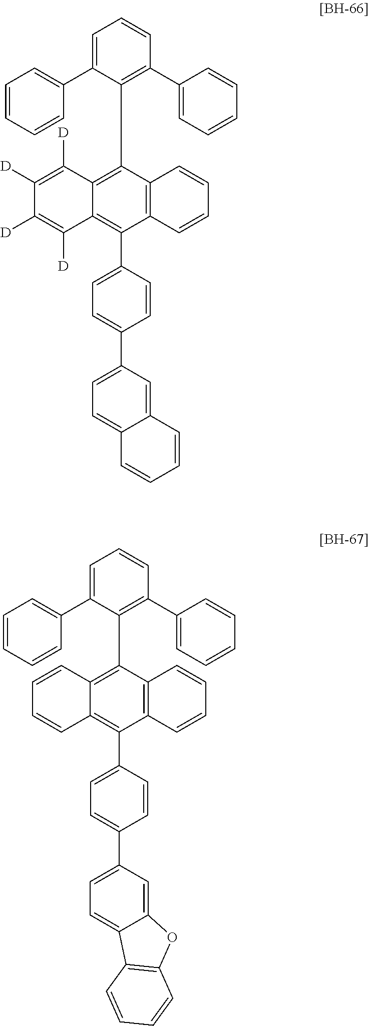 [SMILES: [2H]c1c([2H])c([2H])c2c(-c3c(-c4ccccc4)cccc3-c3ccccc3)c3ccccc3c(-c3ccc(-c4ccc5ccccc5c4)cc3)c2c1[2H] ; c1ccc(-c2cccc(-c3ccccc3)c2-c2c3ccccc3c(-c3ccc(-c4ccc5c(c4)oc4ccccc45)cc3)c3ccccc23)cc1]

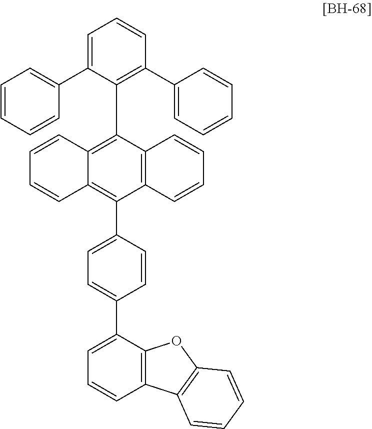 [SMILES: c1ccc(-c2cccc(-c3ccccc3)c2-c2c3ccccc3c(-c3ccc(-c4cccc5c4oc4ccccc45)cc3)c3ccccc23)cc1]

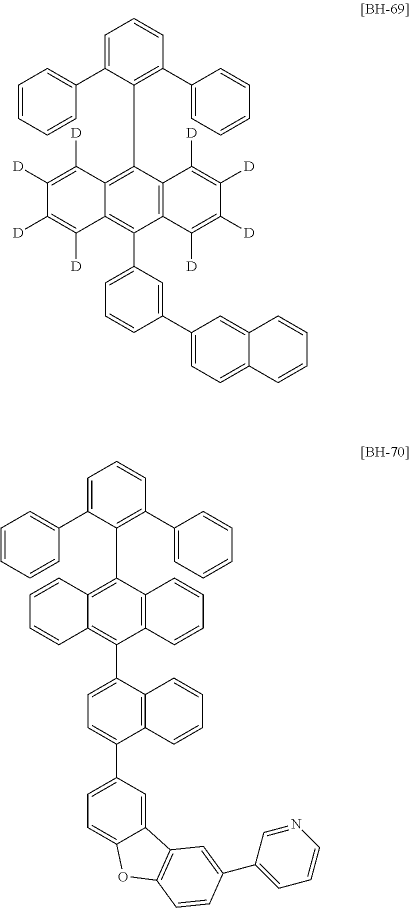 [SMILES: [2H]c1c([2H])c([2H])c2c(-c3c(-c4ccccc4)cccc3-c3ccccc3)c3c([2H])c([2H])c([2H])c([2H])c3c(-c3cccc(-c4ccc5ccccc5c4)c3)c2c1[2H] ; c1ccc(-c2cccc(-c3ccccc3)c2-c2c3ccccc3c(-c3ccc(-c4ccc5oc6ccc(-c7cccnc7)cc6c5c4)c4ccccc34)c3ccccc23)cc1]

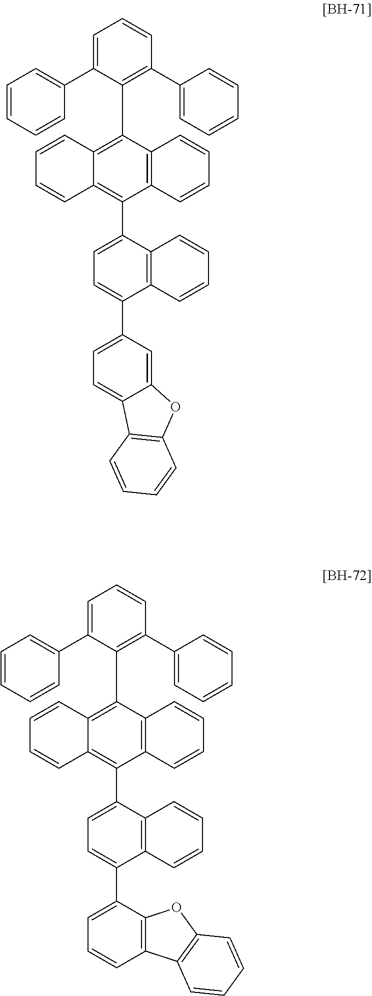 [SMILES: c1ccc(-c2cccc(-c3ccccc3)c2-c2c3ccccc3c(-c3ccc(-c4ccc5c(c4)oc4ccccc45)c4ccccc34)c3ccccc23)cc1 ; c1ccc(-c2cccc(-c3ccccc3)c2-c2c3ccccc3c(-c3ccc(-c4cccc5c4oc4ccccc45)c4ccccc34)c3ccccc23)cc1]

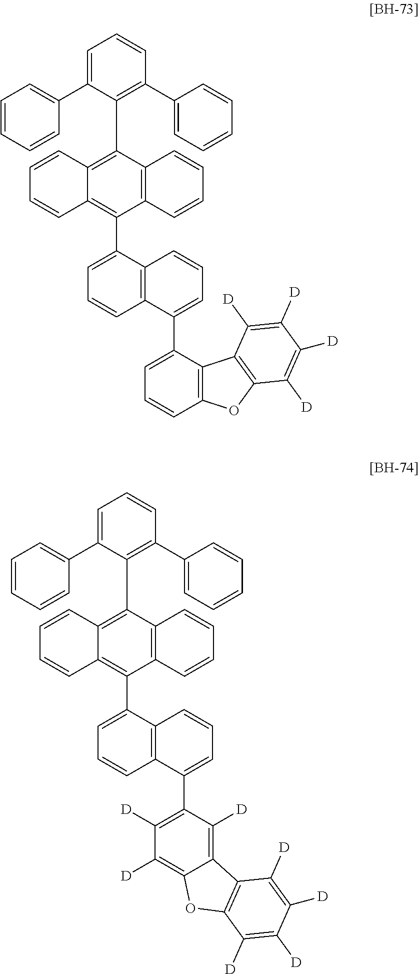 [SMILES: [2H]c1c([2H])c([2H])c2c(oc3cccc(-c4cccc5c(-c6c7ccccc7c(-c7c(-c8ccccc8)cccc7-c7ccccc7)c7ccccc67)cccc45)c32)c1[2H] ; [2H]c1c([2H])c([2H])c2c(oc3c([2H])c([2H])c(-c4cccc5c(-c6c7ccccc7c(-c7c(-c8ccccc8)cccc7-c7ccccc7)c7ccccc67)cccc45)c([2H])c32)c1[2H]]

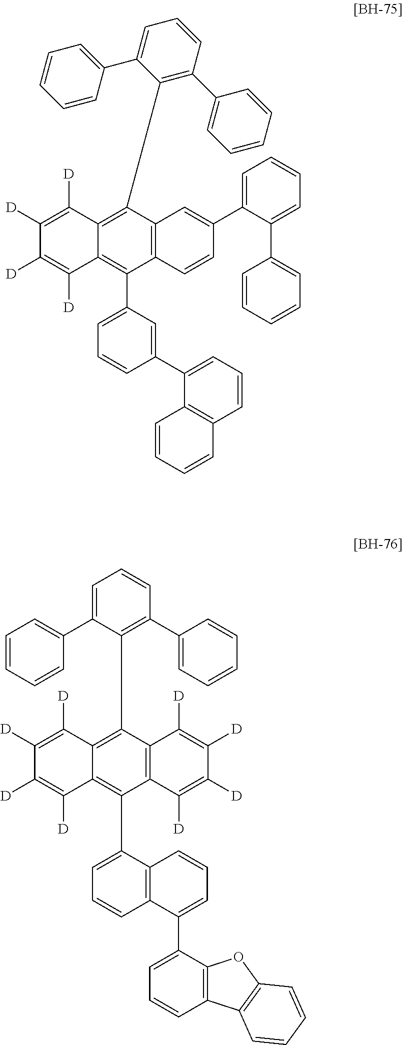 [SMILES: [2H]c1c([2H])c([2H])c2c(-c3c(-c4ccccc4)cccc3-c3ccccc3)c3cc(-c4ccccc4-c4ccccc4)ccc3c(-c3cccc(-c4cccc5ccccc45)c3)c2c1[2H] ; [2H]c1c([2H])c([2H])c2c(-c3cccc4c(-c5cccc6c5oc5ccccc56)cccc34)c3c([2H])c([2H])c([2H])c([2H])c3c(-c3c(-c4ccccc4)cccc3-c3ccccc3)c2c1[2H]]

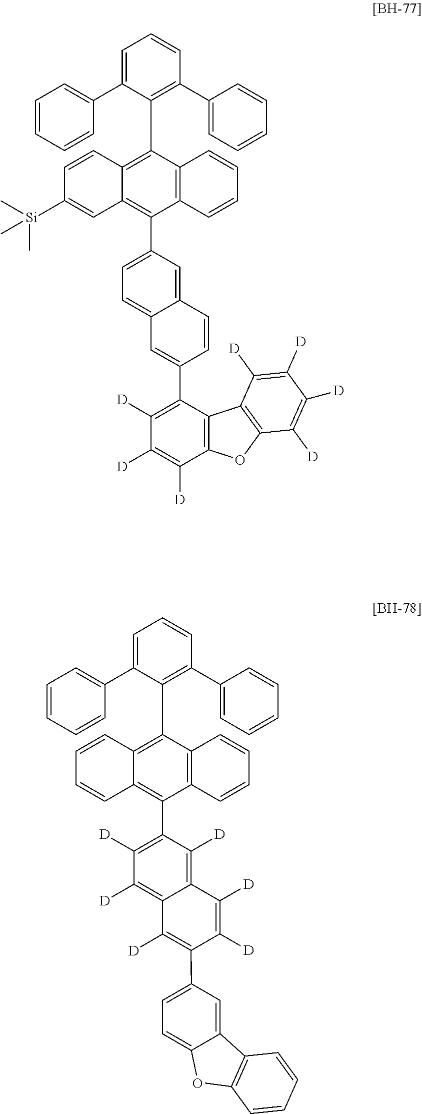 [SMILES: [2H]c1c([2H])c([2H])c2c(oc3c([2H])c([2H])c([2H])c(-c4ccc5cc(-c6c7ccccc7c(-c7c(-c8ccccc8)cccc7-c7ccccc7)c7ccc([Si](C)(C)C)cc67)ccc5c4)c32)c1[2H] ; [2H]c1c(-c2c3ccccc3c(-c3c(-c4ccccc4)cccc3-c3ccccc3)c3ccccc23)c([2H])c2c([2H])c([2H])c(-c3ccc4oc5ccccc5c4c3)c([2H])c2c1[2H]]

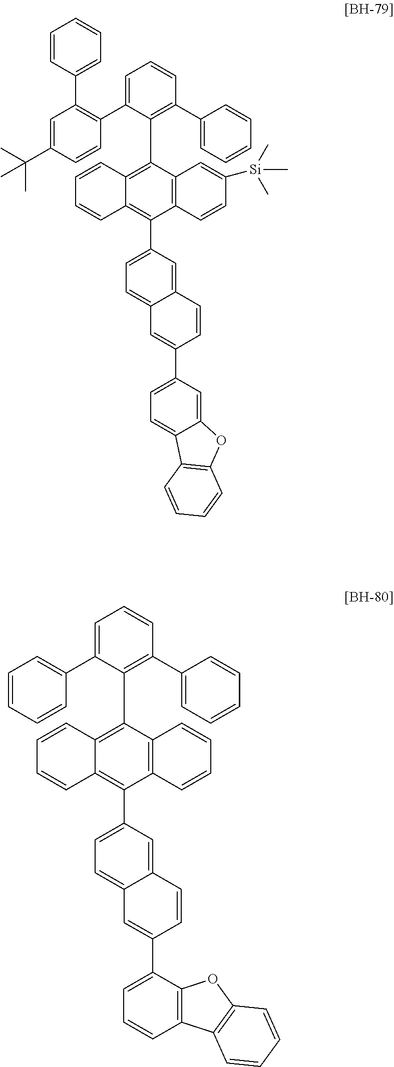 [SMILES: CC(C)(C)c1ccc(-c2cccc(-c3ccccc3)c2-c2c3ccccc3c(-c3ccc4cc(-c5ccc6c(c5)oc5ccccc56)ccc4c3)c3ccc([Si](C)(C)C)cc23)c(-c2ccccc2)c1 ; c1ccc(-c2cccc(-c3ccccc3)c2-c2c3ccccc3c(-c3ccc4cc(-c5cccc6c5oc5ccccc56)ccc4c3)c3ccccc23)cc1]

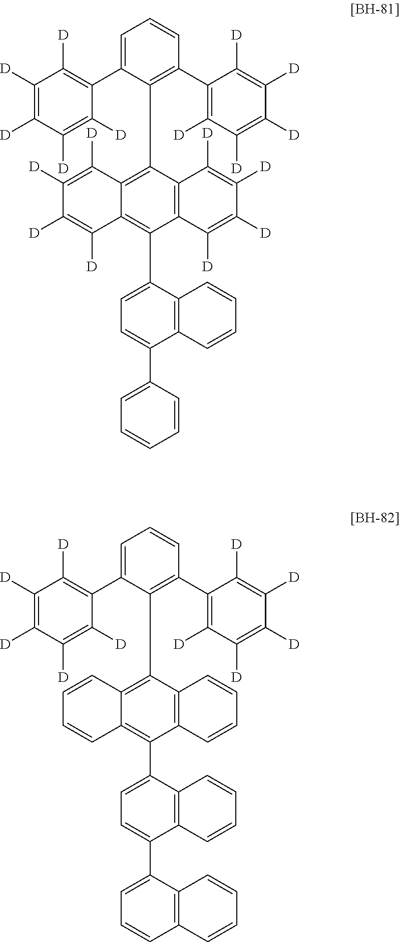 [SMILES: [2H]c1c([2H])c([2H])c(-c2cccc(-c3c([2H])c([2H])c([2H])c([2H])c3[2H])c2-c2c3c([2H])c([2H])c([2H])c([2H])c3c(-c3ccc(-c4ccccc4)c4ccccc34)c3c([2H])c([2H])c([2H])c([2H])c23)c([2H])c1[2H] ; [2H]c1c([2H])c([2H])c(-c2cccc(-c3c([2H])c([2H])c([2H])c([2H])c3[2H])c2-c2c3ccccc3c(-c3ccc(-c4cccc5ccccc45)c4ccccc34)c3ccccc23)c([2H])c1[2H]]

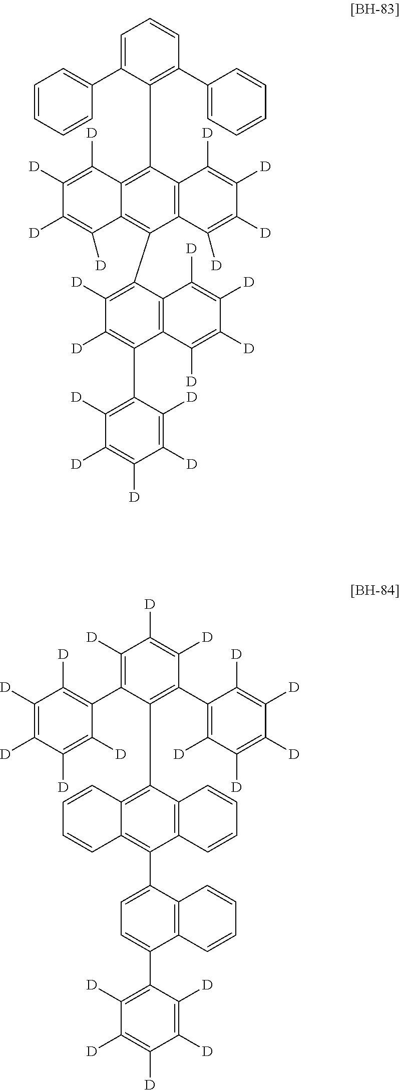 [SMILES: [2H]c1c([2H])c([2H])c(-c2c([2H])c([2H])c(-c3c4c([2H])c([2H])c([2H])c([2H])c4c(-c4c(-c5ccccc5)cccc4-c4ccccc4)c4c([2H])c([2H])c([2H])c([2H])c34)c3c([2H])c([2H])c([2H])c([2H])c23)c([2H])c1[2H] ; [2H]c1c([2H])c([2H])c(-c2c([2H])c([2H])c([2H])c(-c3c([2H])c([2H])c([2H])c([2H])c3[2H])c2-c2c3ccccc3c(-c3ccc(-c4c([2H])c([2H])c([2H])c([2H])c4[2H])c4ccccc34)c3ccccc23)c([2H])c1[2H]]

An organic light-emitting diode according to the present disclosure employs a compound represented by Chemical Formula A as a dopant and a compound represented by any one of Chemical Formulas 3, 4, and 3-1 to 4-3 as a dopant, demonstrating high efficiency and longevity.

Throughout the description of the present disclosure, the phrase “(organic layer) includes at least one organic compound” may be construed to mean that “(organic layer) may include a single organic compound species or two or more different species of organic compounds falling within the scope of the present disclosure”.

Moreover, the organic light-emitting diode of the present disclosure may include at least one of a hole injection layer, a hole transport layer, a functional layer capable of both hole injection and hole transport, an electron transport layer, and an electron injection layer, in addition to the light-emitting layer.

In the present disclosure, as plural neighboring substituents within the same category (e.g., among R, R′, R₁ to R₈, between R₁₂ and R₁₃, between R₁₄ and R₁₅, and between R₁₆ and R₁₇, etc. in Chemical Formulas A, 3, 4, 3-1 to 4-3 or between R₂₁ or R₂₅ and R₂₆ in Chemical Formulas 3-1 and 4-1) are connected to each other, the aliphatic or aromatic mono- or polycyclic ring thus formed may bear a heteroatom such as N, NR′″, O, S, Si, Ge, etc., as a ring member, wherein R′″ are as defined for R₁₀ to R₁₇, with at least one hydrogen atom in the ring being substitutable with a deuterium atom.

Concrete examples of the dopant compound useful for the light-emitting layer in the organic light-emitting diode according to the present disclosure include, but are not limited to, the following compounds [BD-1] to [BD-48]:

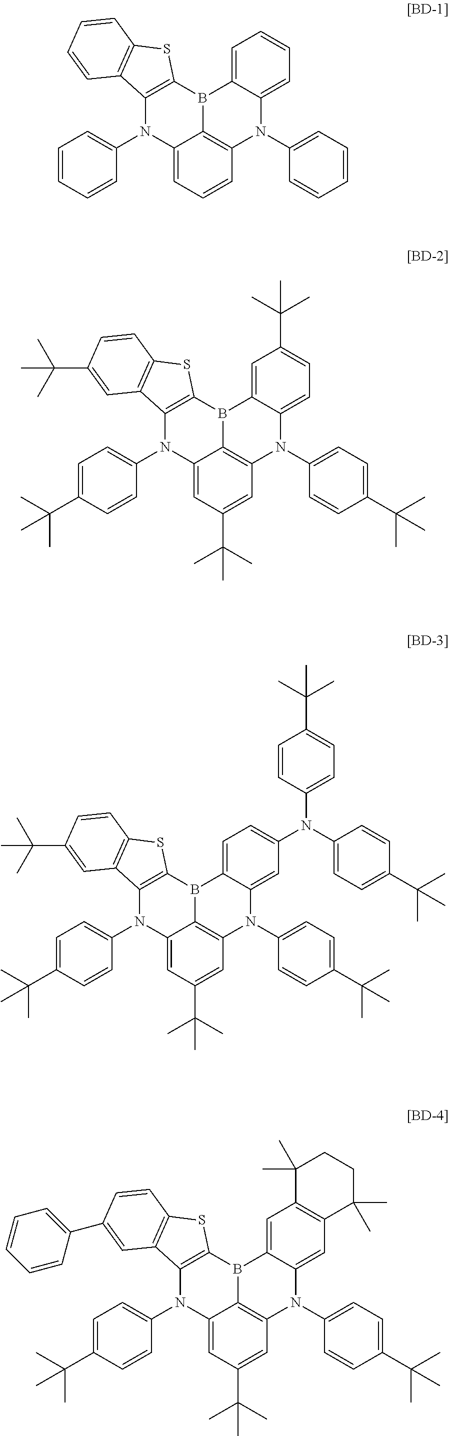

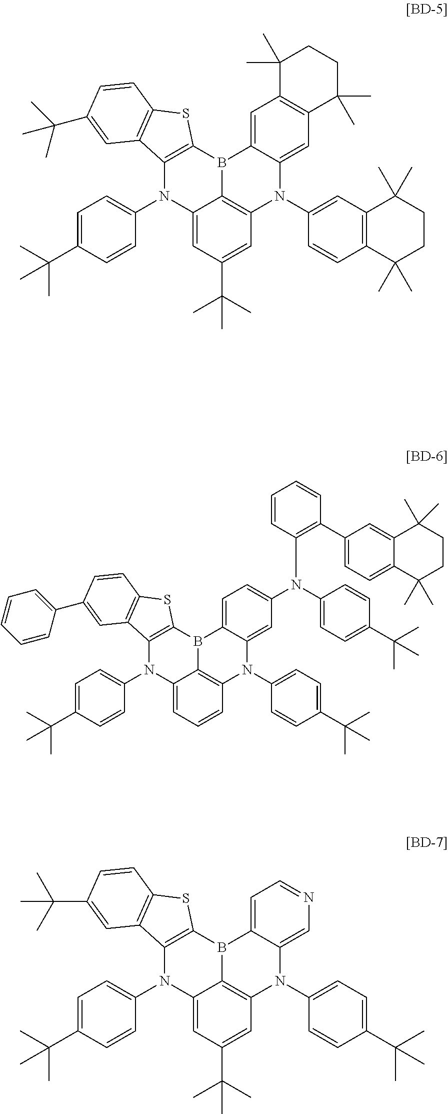 [SMILES: CC(C)(C)c1ccc(N2c3cc(C(C)(C)C)cc4c3B(c3cc5c(cc3N4c3ccc4c(c3)C(C)(C)CCC4(C)C)C(C)(C)CCC5(C)C)c3sc4ccc(C(C)(C)C)cc4c32)cc1 ; CC(C)(C)c1ccc(N(c2ccc3c(c2)N(c2ccc(C(C)(C)C)cc2)c2cccc4c2B3c2sc3ccc(-c5ccccc5)cc3c2N4c2ccc(C(C)(C)C)cc2)c2ccccc2-c2ccc3c(c2)C(C)(C)CCC3(C)C)cc1 ; CC(C)(C)c1ccc(N2c3cnccc3B3c4sc5ccc(C(C)(C)C)cc5c4N(c4ccc(C(C)(C)C)cc4)c4cc(C(C)(C)C)cc2c43)cc1]

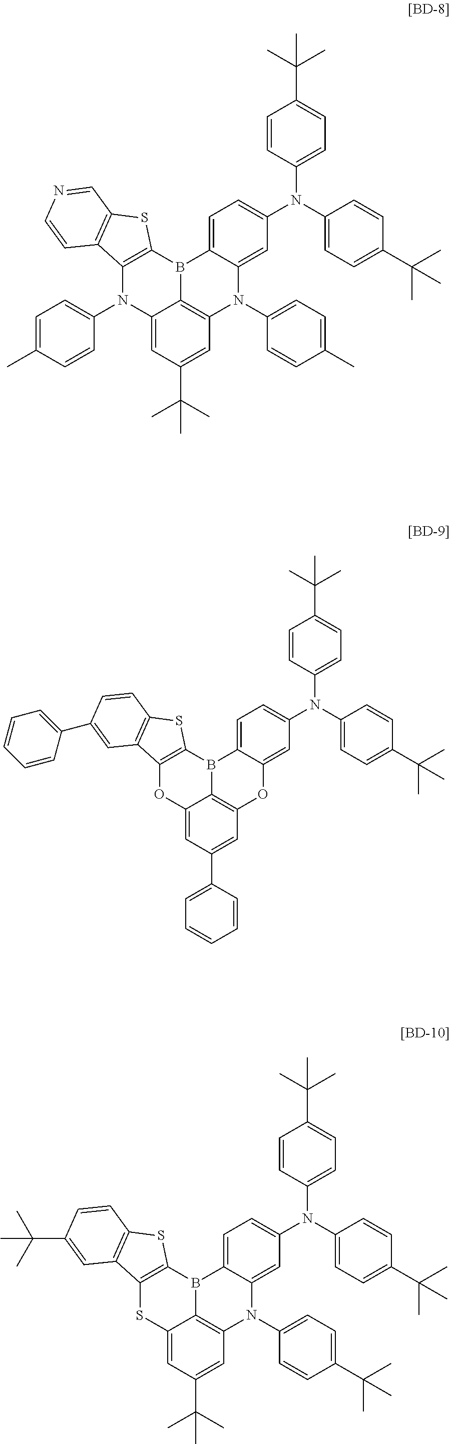

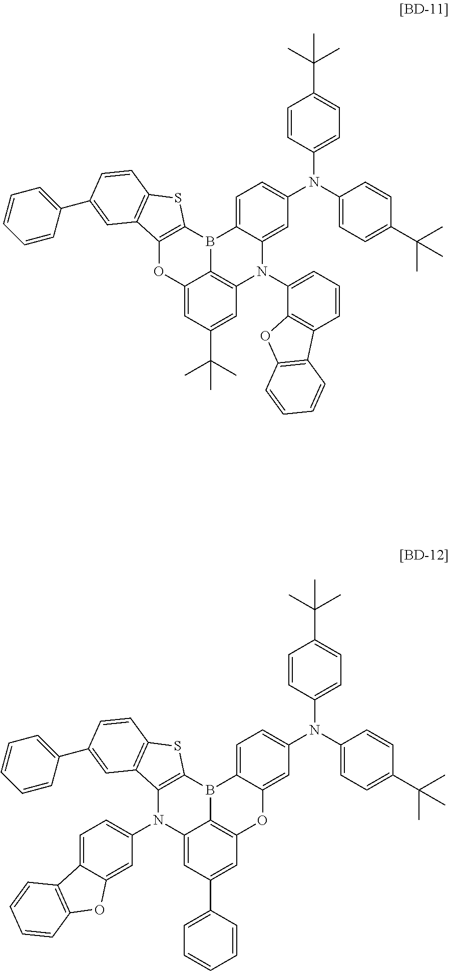 [SMILES: CC(C)(C)c1ccc(N(c2ccc(C(C)(C)C)cc2)c2ccc3c(c2)N(c2cccc4c2oc2ccccc24)c2cc(C(C)(C)C)cc4c2B3c2sc3ccc(-c5ccccc5)cc3c2O4)cc1 ; CC(C)(C)c1ccc(N(c2ccc(C(C)(C)C)cc2)c2ccc3c(c2)Oc2cc(-c4ccccc4)cc4c2B3c2sc3ccc(-c5ccccc5)cc3c2N4c2ccc3c(c2)oc2ccccc23)cc1]

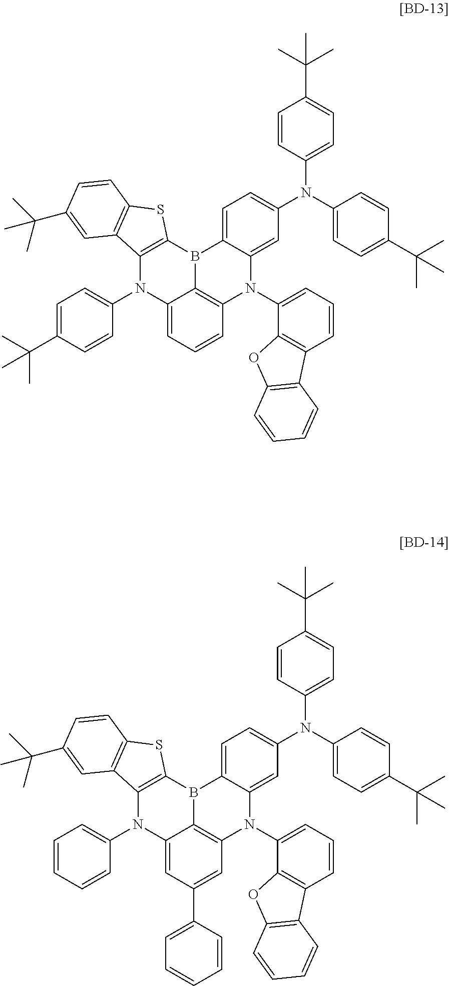

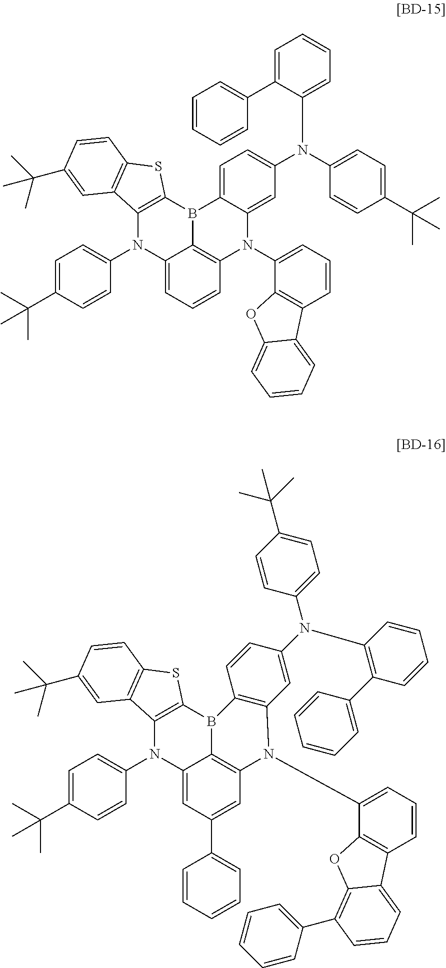

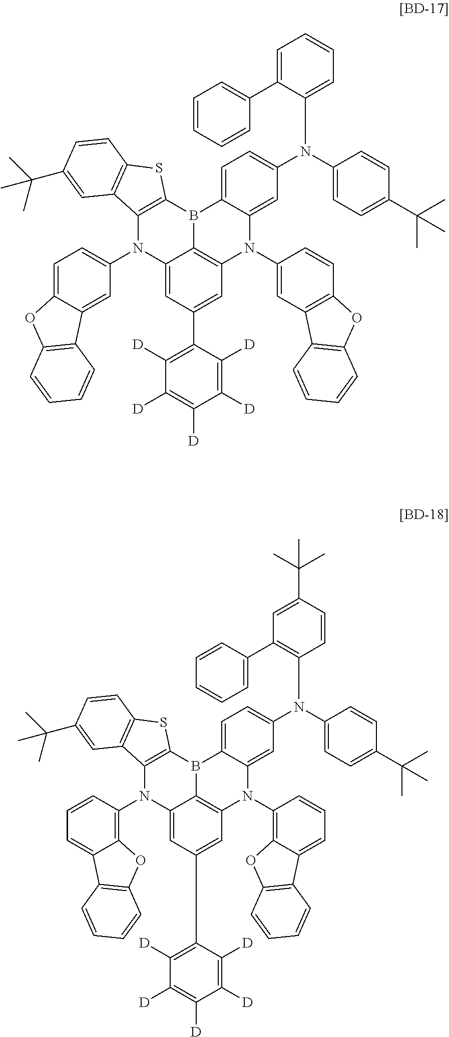

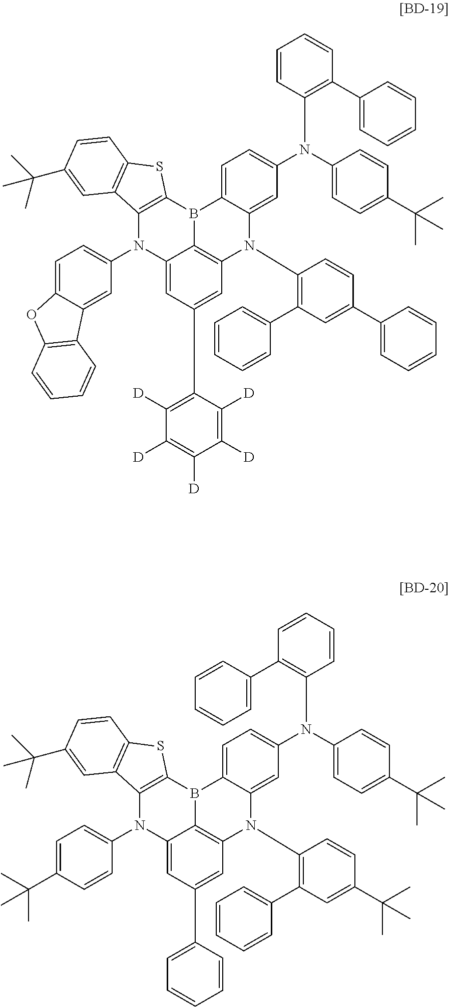

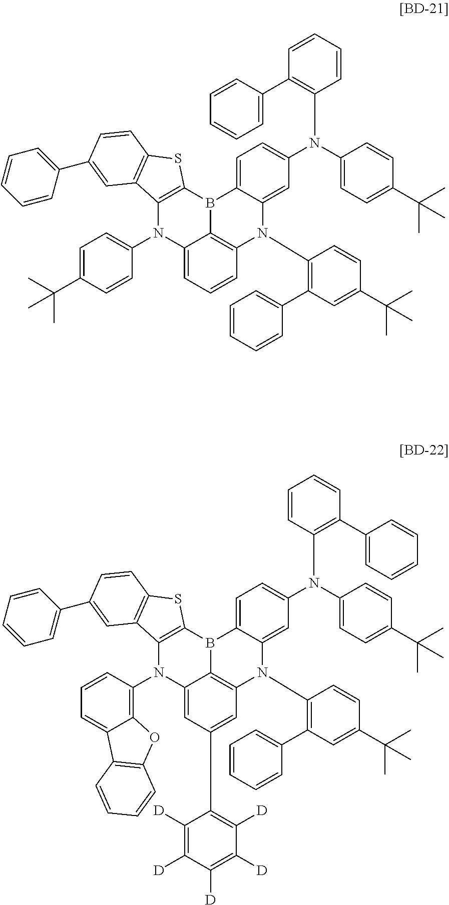 [SMILES: CC(C)(C)c1ccc(N(c2ccc3c(c2)N(c2ccc(C(C)(C)C)cc2-c2ccccc2)c2cccc4c2B3c2sc3ccc(-c5ccccc5)cc3c2N4c2ccc(C(C)(C)C)cc2)c2ccccc2-c2ccccc2)cc1 ; [2H]c1c([2H])c([2H])c(-c2cc3c4c(c2)N(c2cccc5c2oc2ccccc25)c2c(sc5ccc(-c6ccccc6)cc25)B4c2ccc(N(c4ccc(C(C)(C)C)cc4)c4ccccc4-c4ccccc4)cc2N3c2ccc(C(C)(C)C)cc2-c2ccccc2)c([2H])c1[2H]]

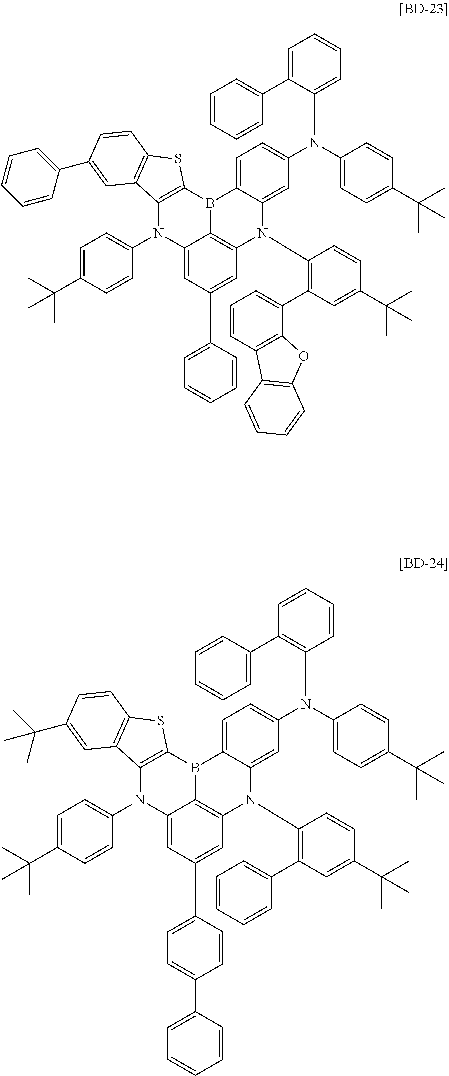

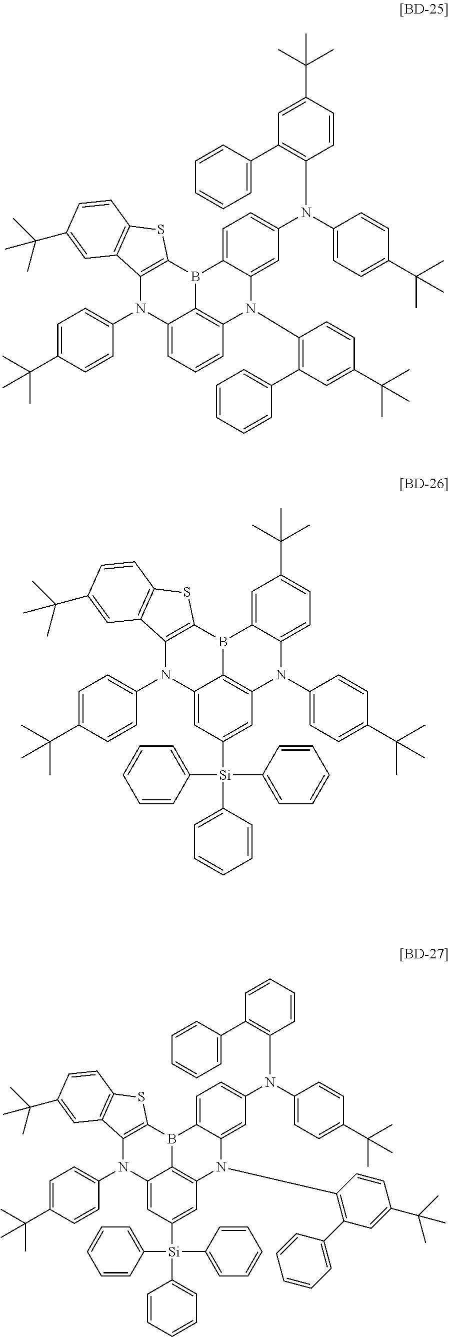

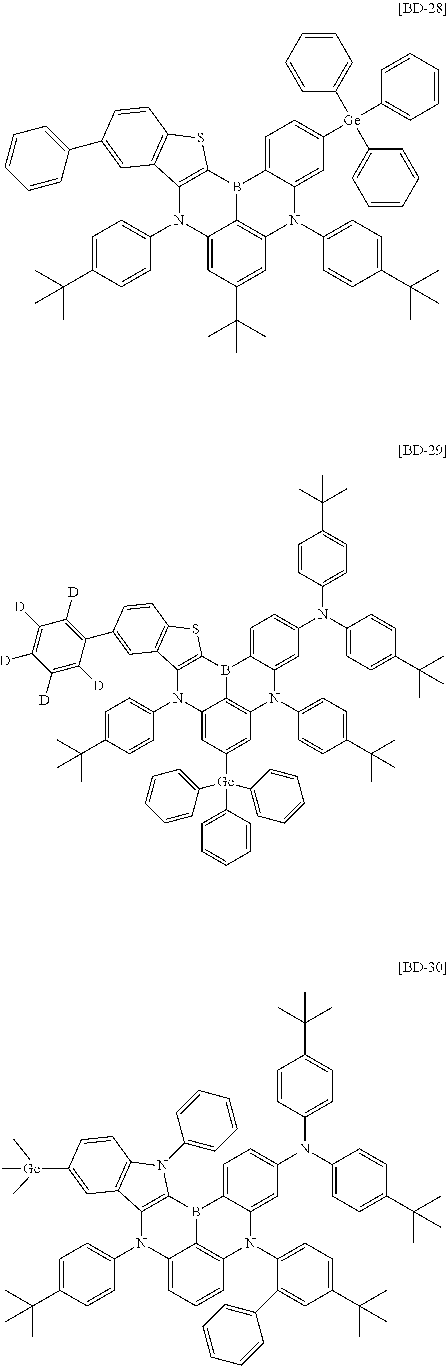

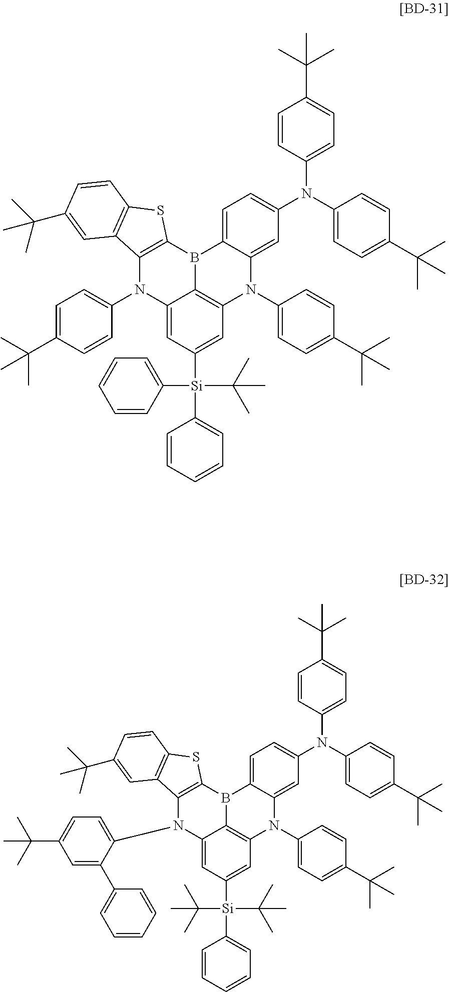

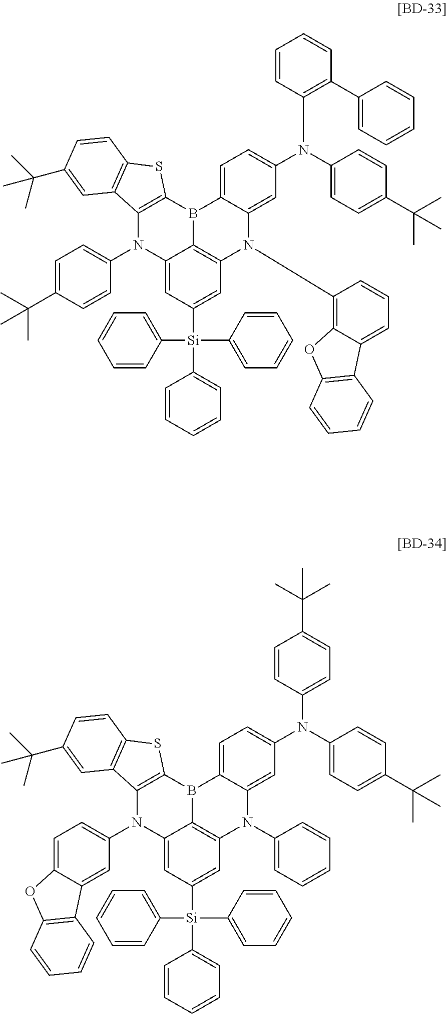

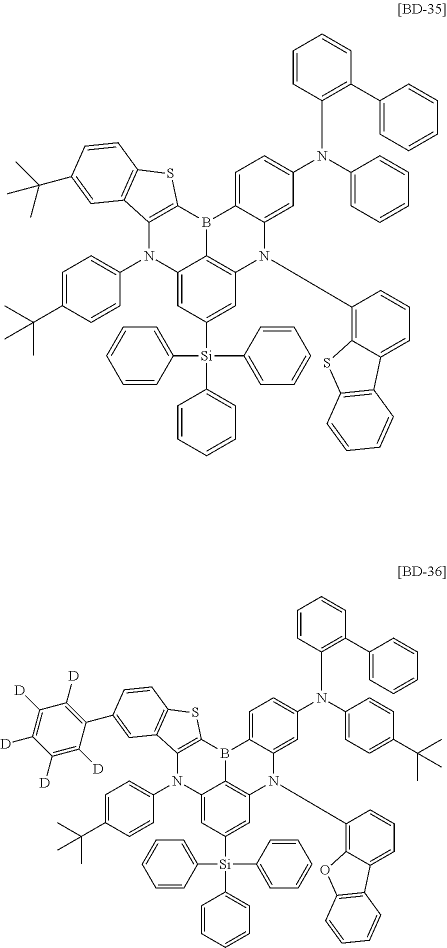 [SMILES: CC(C)(C)c1ccc(N2c3cc([Si](c4ccccc4)(c4ccccc4)c4ccccc4)cc4c3B(c3ccc(N(c5ccccc5)c5ccccc5-c5ccccc5)cc3N4c3cccc4c3sc3ccccc34)c3sc4ccc(C(C)(C)C)cc4c32)cc1 ; [2H]c1c([2H])c([2H])c(-c2ccc3sc4c(c3c2)N(c2ccc(C(C)(C)C)cc2)c2cc([Si](c3ccccc3)(c3ccccc3)c3ccccc3)cc3c2B4c2ccc(N(c4ccc(C(C)(C)C)cc4)c4ccccc4-c4ccccc4)cc2N3c2cccc3c2oc2ccccc23)c([2H])c1[2H]]

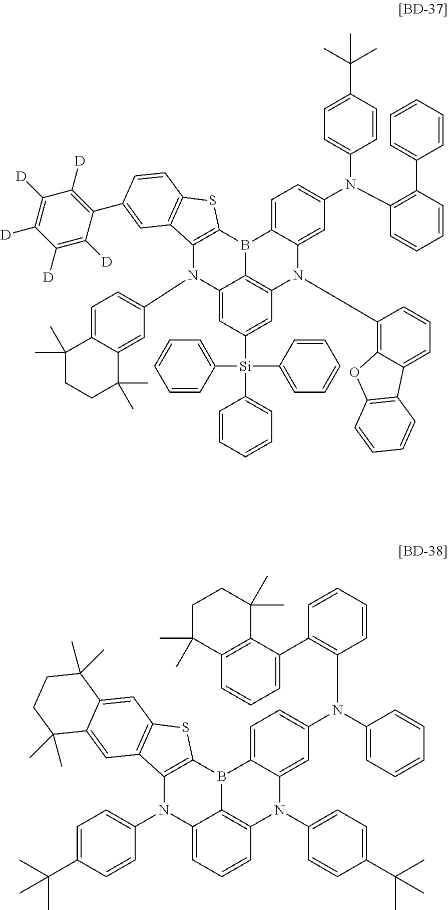 [SMILES: [2H]c1c([2H])c([2H])c(-c2ccc3sc4c(c3c2)N(c2ccc3c(c2)C(C)(C)CCC3(C)C)c2cc([Si](c3ccccc3)(c3ccccc3)c3ccccc3)cc3c2B4c2ccc(N(c4ccc(C(C)(C)C)cc4)c4ccccc4-c4ccccc4)cc2N3c2cccc3c2oc2ccccc23)c([2H])c1[2H] ; CC(C)(C)c1ccc(N2c3cc(N(c4ccccc4)c4ccccc4-c4cccc5c4C(C)(C)CCC5(C)C)ccc3B3c4sc5cc6c(cc5c4N(c4ccc(C(C)(C)C)cc4)c4cccc2c43)C(C)(C)CCC6(C)C)cc1]

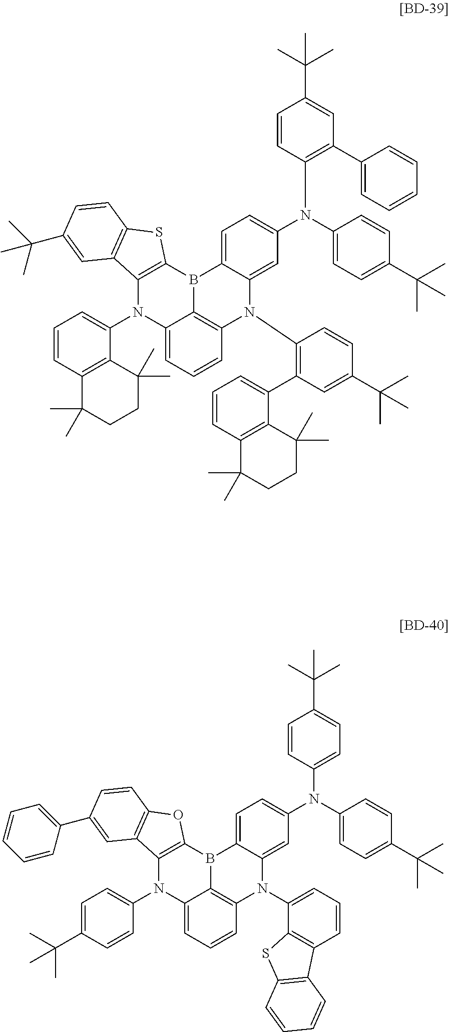 [SMILES: CC(C)(C)c1ccc(N(c2ccc3c(c2)N(c2ccc(C(C)(C)C)cc2-c2cccc4c2C(C)(C)CCC4(C)C)c2cccc4c2B3c2sc3ccc(C(C)(C)C)cc3c2N4c2cccc3c2C(C)(C)CCC3(C)C)c2ccc(C(C)(C)C)cc2-c2ccccc2)cc1 ; CC(C)(C)c1ccc(N(c2ccc(C(C)(C)C)cc2)c2ccc3c(c2)N(c2cccc4c2sc2ccccc24)c2cccc4c2B3c2oc3ccc(-c5ccccc5)cc3c2N4c2ccc(C(C)(C)C)cc2)cc1]

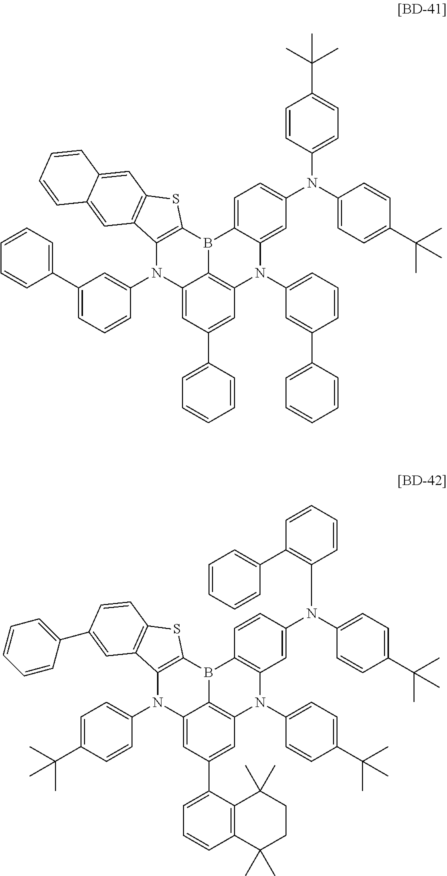 [SMILES: CC(C)(C)c1ccc(N(c2ccc(C(C)(C)C)cc2)c2ccc3c(c2)N(c2cccc(-c4ccccc4)c2)c2cc(-c4ccccc4)cc4c2B3c2sc3cc5ccccc5cc3c2N4c2cccc(-c3ccccc3)c2)cc1 ; CC(C)(C)c1ccc(N(c2ccc3c(c2)N(c2ccc(C(C)(C)C)cc2)c2cc(-c4cccc5c4C(C)(C)CCC5(C)C)cc4c2B3c2sc3ccc(-c5ccccc5)cc3c2N4c2ccc(C(C)(C)C)cc2)c2ccccc2-c2ccccc2)cc1]

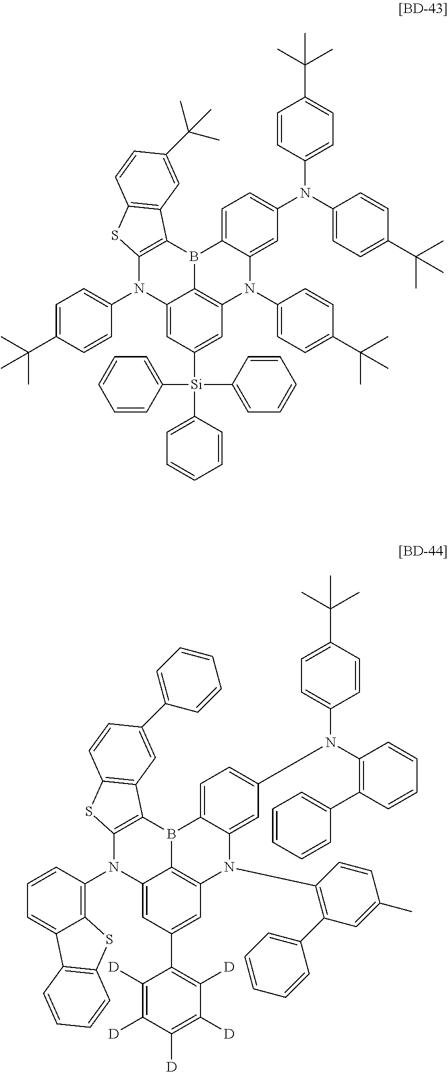 [SMILES: CC(C)(C)c1ccc(N(c2ccc(C(C)(C)C)cc2)c2ccc3c(c2)N(c2ccc(C(C)(C)C)cc2)c2cc([Si](c4ccccc4)(c4ccccc4)c4ccccc4)cc4c2B3c2c(sc3ccc(C(C)(C)C)cc23)N4c2ccc(C(C)(C)C)cc2)cc1 ; [2H]c1c([2H])c([2H])c(-c2cc3c4c(c2)N(c2cccc5c2sc2ccccc25)c2sc5ccc(-c6ccccc6)cc5c2B4c2ccc(N(c4ccc(C(C)(C)C)cc4)c4ccccc4-c4ccccc4)cc2N3c2ccc(C)cc2-c2ccccc2)c([2H])c1[2H]]

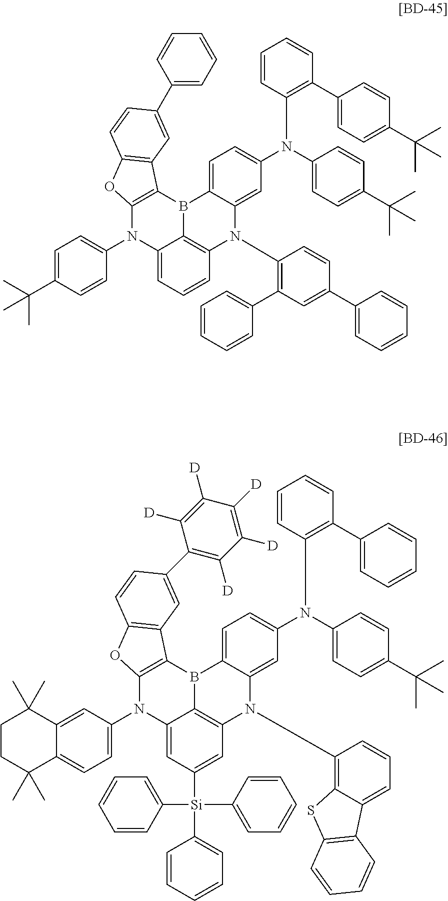 [SMILES: CC(C)(C)c1ccc(-c2ccccc2N(c2ccc(C(C)(C)C)cc2)c2ccc3c(c2)N(c2ccc(-c4ccccc4)cc2-c2ccccc2)c2cccc4c2B3c2c(oc3ccc(-c5ccccc5)cc23)N4c2ccc(C(C)(C)C)cc2)cc1 ; [2H]c1c([2H])c([2H])c(-c2ccc3oc4c(c3c2)B2c3ccc(N(c5ccc(C(C)(C)C)cc5)c5ccccc5-c5ccccc5)cc3N(c3cccc5c3sc3ccccc35)c3cc([Si](c5ccccc5)(c5ccccc5)c5ccccc5)cc(c32)N4c2ccc3c(c2)C(C)(C)CCC3(C)C)c([2H])c1[2H]]

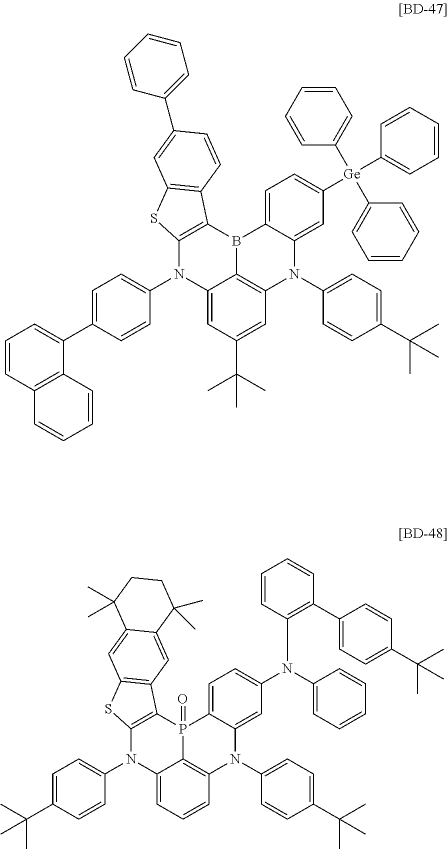

The content of the dopant in the light-emitting diode may range from about 0.01 to about 20 parts by weight, based on 100 parts by weight of the host, but with no limitations thereto.

Furthermore, the light-emitting layer may additionally contain various hosts and dopant materials in addition to the dopant and host.

Below, the organic light-emitting diode of the present disclosure is explained with reference to the drawing.

The FIG. 1s a schematic cross-sectional view of the structure of an organic light-emitting diode according to an embodiment of the present disclosure.

As shown in The FIGURE, the organic light-emitting diode according to an embodiment of the present disclosure comprises an anode (20), a hole transport layer (40), an organic light-emitting layer (50) containing a host and a dopant, an electron transport layer (60), and a cathode (80), wherein the anode and the cathode serve as a first electrode and a second electrode, respectively, with the interposition of the hole transport layer between the anode and the light-emitting layer, and the electron transport layer between the light-emitting layer and the cathode.

Furthermore, the organic light-emitting diode according to an embodiment of the present disclosure may include a hole injection layer (30) between the anode (20) and the hole transport layer (40), and an electron injection layer (70) between the electron transport layer (60) and the cathode (80).

Reference is made to The FIGURE with regard to the organic light emitting diode of the present disclosure and the fabrication method therefor.

First, a substrate (10) is coated with an anode electrode material to form an anode (20). So long as it is used in a typical organic electroluminescence device, any substrate may be used as the substrate (10). Preferable is an organic substrate or transparent plastic substrate that exhibits excellent transparency, surface smoothness, ease of handling, and waterproofness. As the anode electrode material, indium tin oxide (ITO), indium zinc oxide (IZO), tin oxide (SnO₂), or zinc oxide (ZnO), which are transparent and superior in terms of conductivity, may be used.

A hole injection layer material is applied on the anode (20) by thermal deposition in a vacuum or by spin coating to form a hole injection layer (30). Subsequently, thermal deposition in a vacuum or by spin coating may also be conducted to form a hole transport layer (40) with a hole transport layer material on the hole injection layer (30).

So long as it is typically used in the art, any material may be selected for the hole injection layer without particular limitations thereto. Examples include, but are not limited to, 2-TNATA [4,4′,4″-tris(2-naphthylphenyl-phenylamino)-triphenylamine], NPD [N,N′-di(1-naphthyl)-N,N′-diphenylbenzidine)], TPD [N,N′-diphenyl-N,N′-bis(3-methylphenyl)-1,1′-biphenyl-4,4′-diamine], and DNTPD [N,N′-diphenyl-N,N′-bis-[4-(phenyl-m-tolyl-amino)-phenyl]-biphenyl-4,4′-diamine].

Any material that is typically used in the art may be selected for the hole transport layer without particular limitations thereto. Examples include, but are not limited to, N,N′-bis(3-methylphenyl)-N,N′-diphenyl-[1,1-biphenyl]-4,4′-diamine (TPD) and N,N′-di(naphthalen-1-yl)-N,N′-diphenylbenzidine (a-NPD).

In an embodiment of the present disclosure, an electron blocking layer (electron barrier layer) may be additionally disposed on the hole transport layer. Functioning to prevent the electrons injected from the electron injection layer from entering the hole transport layer from the light-emitting layer, the electron blocking layer is adapted to increase the life span and luminous efficiency of the diode. The electron blocking layer may be formed at a suitable position between the light emitting layer and the hole injection layer. Particularly, the electron blocking layer may be formed between the light emitting layer and the hole transport layer.

Next, the light-emitting layer (50) may be deposited on the hole transport layer (40) or the electron blocking layer by deposition in a vacuum or by spin coating.

Herein, the light-emitting layer may contain a host and a dopant and the materials are as described above.

In some embodiments of the present disclosure, the light-emitting layer particularly ranges in thickness from 50 to 2,000 Å.

In the present disclosure, two or more different host compounds including the anthracene compound represented by Chemical Formula A and at least one species of host compound other than the anthracene compound may be mixed or stacked in the light-emitting layer.

Moreover, two or more different dopant compounds including the compound represented by any one of Chemical Formulas 3, 4, and 3-1 to 4-3 and at least one species of dopant compound other than the dopant compounds represented by the chemical formulas may be mixed or stacked in the light-emitting layer.

Here, as an example of using stacked light-emitting layers, the present disclosure may employ a light-emitting layer in a bi- or multilayer structure including: a first light-emitting layer containing a host represented by Chemical Formula A and a dopant represented by any one of Chemical Formulas 3, 4, and 3-1 to 4-3; and a second light-emitting layer.

That is, the light emitting layer according to the present disclosure may be a stacked light emitting layer in a bi- or multilayer structure including: a first light emitting layer containing a host represented by Chemical Formula A and a dopant represented by any one of Chemical Formulas 3, 4, and 3-1 to 4-3; and a second light emitting layer.

Here, the second light emitting layer is preferably different from the first light emitting layer in that at least one of the host compound and dopant compound employed in the first light emitting layer is not applied in the second light emitting layer.

That is, the light emitting layer according to the present disclosure has a bi- or multilayer structure in which the first light emitting layer contains: the host represented by Chemical Formula A or B; and the dopant represented by any one of Chemical Formulas 3, 4, and 3-1 to 4-3 and the second light emitting layer may contain a compound, different from the compound represented by Chemical Formula A, as a host; and a compound, different from the compound represented by any one of Chemical Formulas 3, 4, and 3-1 to 4-3, as a dopant.

Also, the second light emitting layer of the present disclosure may employ as a host a compound represented by Chemical Formula A that is different from the compound used as a host in the first light emitting layer. Moreover, a compound represented by any one of Chemical Formulas 3 to 4 and 3-1 to 4-3, but different from the compound used as a dopant in the first light emitting layer, may be used as a dopant in a second light emitting layer. Thus, a second light emitting layer different from the first light emitting layer may be deposited.

For example, the first light emitting layer may contain compound BH-3 as a host and compound BD-36 as a dopant while the second light emitting layer may contain compound BH-6 as a host and compound BD-24 as a dopant. The first light emitting layer may contain compound BH-13 as a host and compound BD-14 as a dopant while the second light emitting layer may contain compound BH-6 as a host and a compound, different from that represented by any one of Chemical Formulas 3, 4, and 3-1 to 4-3, as a dopant. In another example, compound BH-6 according to the present disclosure is used as a host and BD-20 as a dopant in the first light emitting layer while the second light emitting layer employes a compound, different from that represented by Chemical Formula A, as a host and a compound represented by any one of Chemical Formulas 3, 4, and 3-1 to 4-3 as a dopant.

In the present disclosure, a hole barrier layer may be additionally formed above the light-emitting layer. Functioning to prevent the holes injected from the hole injection layer from passing through the light-emitting layer and entering the electron transport layer, the hole barrier layer is to enhance the life span and efficiency of the diode and may be formed at an appropriate part between the light-emitting layer and the electron transport layer.

On the light-emitting layer, an electron transport layer (60) is deposited using a vacuum deposition method or spin coating method.

A material for use in the electron transport layer functions to stably carry the electrons injected from the electron injection electrode (cathode), and may be an electron transport material known in the art. Examples of the electron transport material known in the art include quinoline derivatives, particularly, tris(8-quinolinolate)aluminum (Alq₃), Liq, TAZ, BAlq, beryllium bis(benzoquinolin-10-olate) (Bebq₂), Compound 201, Compound 202, BCP, and oxadiazole derivatives such as PBD, BMD, and BND, but are not limited thereto:

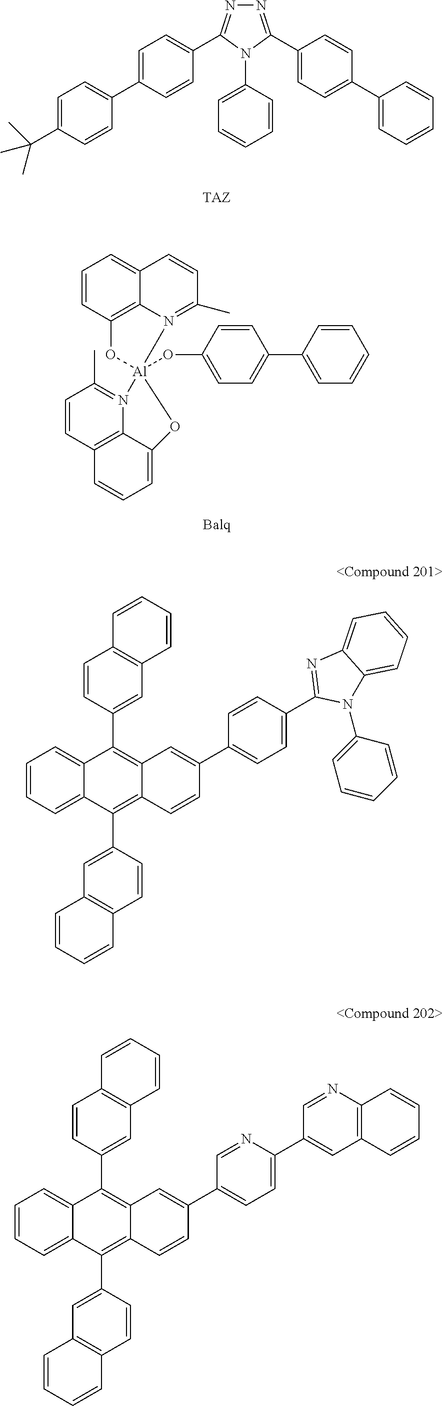

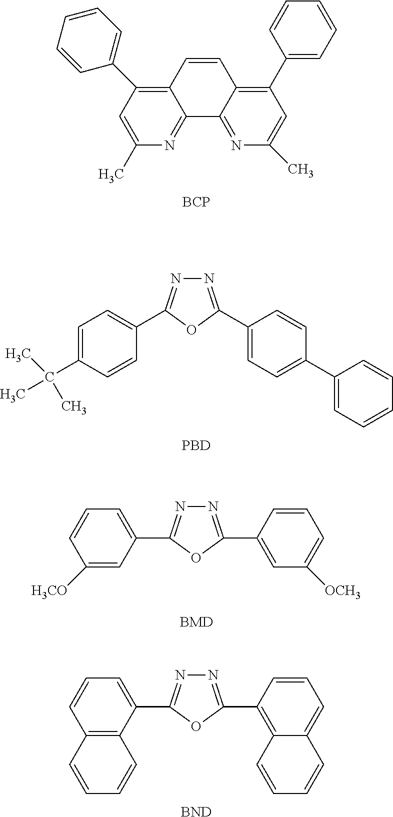

In the organic light emitting diode of the present disclosure, an electron injection layer (EIL) that functions to facilitate electron injection from the cathode may be deposited on the electron transport layer. The material for the EIL is not particularly limited.

Any material that is conventionally used in the art can be available for the electron injection layer without particular limitations. Examples include CsF, NaF, LiF, Li₂O, and BaO. Deposition conditions for the electron injection layer may vary, depending on compounds used, but may be generally selected from condition scopes that are almost the same as for the formation of hole injection layers.

The electron injection layer may range in thickness from about 1 Å to about 100 Å, and particularly from about 3 Å to about 90 Å. Given the thickness range for the electron injection layer, the diode can exhibit satisfactory electron injection properties without actually elevating a driving voltage.

In order to facilitate electron injection, the cathode may be made of a material having a small work function, such as metal or metal alloy such as lithium (Li), magnesium (Mg), calcium (Ca), an alloy aluminum (Al) thereof, aluminum-lithium (Al—Li), magnesium-indium (Mg—In), and magnesium-silver (Mg—Ag). Alternatively, ITO or IZO may be employed to form a transparent cathode for an organic light-emitting diode.

Moreover, the organic light-emitting diode of the present disclosure may further comprise a light-emitting layer containing a blue, green, or red luminescent material that emits radiations in the wavelength range of 380 nm to 800 nm. That is, the light-emitting layer in the present disclosure has a multi-layer structure wherein the blue, green, or red luminescent material may be a fluorescent material or a phosphorescent material.

Furthermore, at least one selected from among the layers may be deposited using a single-molecule deposition process or a solution process.

Here, the deposition process is a process by which a material is vaporized in a vacuum or at a low pressure and deposited to form a layer, and the solution process is a method in which a material is dissolved in a solvent and applied for the formation of a thin film by means of inkjet printing, roll-to-roll coating, screen printing, spray coating, dip coating, spin coating, etc.

Also, the organic light-emitting diode of the present disclosure may be applied to a device selected from among flat display devices, flexible display devices, monochrome or grayscale flat illumination devices, monochrome or grayscale flexible illumination devices, vehicle display devices, and display devices for virtual or augmented reality.

A better understanding of the present disclosure may be obtained through the following examples which are set forth to illustrate, but are not to be construed as limiting the present disclosure.

### EXAMPLES

### Synthesis Example 1. Synthesis of [BH-1]

### Synthesis Example 1-1: Synthesis of A-1

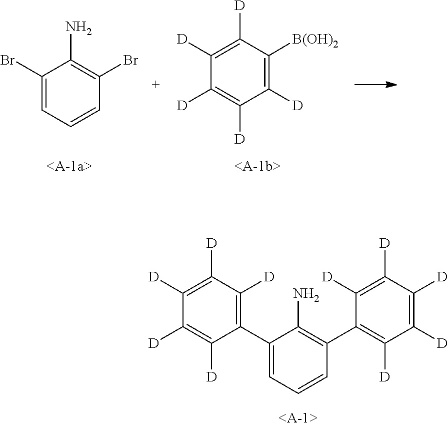 [SMILES: Nc1c(Br)cccc1Br ; [2H]c1c([2H])c([2H])c(B(O)O)c([2H])c1[2H] ; [2H]c1c([2H])c([2H])c(-c2cccc(-c3c([2H])c([2H])c([2H])c([2H])c3[2H])c2N)c([2H])c1[2H]]

In a reactor, <A-1a> (200 g), <A-1b> (222.6 g), tetrakis(triphenylphosphine)palladium(0) (18.4 g), potassium carbonate (220.3 g), toluene (1600 mL), ethanol (760 mL), and distilled water (600 mL) were stirred together overnight under reflux. After the reaction was completed as monitored by TLC, the reaction mixture was cooled to room temperature and then added with distilled water and ethyl acetate. The ethyl acetate layer was isolated, concentrated, purified by column chromatography, and recrystallized to afford <A-1>. (165 g, 81.1%)

### Synthesis Example 1-2 Synthesis of A-2

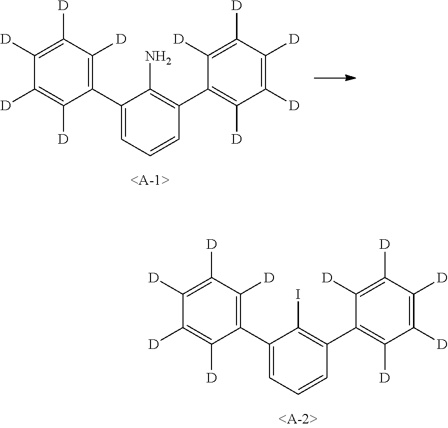 [SMILES: [2H]c1c([2H])c([2H])c(-c2cccc(-c3c([2H])c([2H])c([2H])c([2H])c3[2H])c2N)c([2H])c1[2H] ; [2H]c1c([2H])c([2H])c(-c2cccc(-c3c([2H])c([2H])c([2H])c([2H])c3[2H])c2I)c([2H])c1[2H]]

In a reactor, <A-1> (165 g) was dissolved in acetic acid (1260 mL) and toluene (380 mL), stirred, and maintained at 0° C. for 30 minutes. A solution of sodium nitrite (89.1 g) in distilled water was slowly added over one hour in a dropwise manner, after which a solution of potassium iodide (268.1 g) in distilled water was dropwise added over one hour. The mixture was maintained at 0° C. for 4 hours and then stirred at room temperature for 16 hours. Following slow addition of an aqueous sodium carbonate solution, extraction was conducted with ethyl acetate. The organic layer thus obtained was washed with sodium thiosulfate and dried over sodium sulfate. Purification by column chromatography afforded <A-2>. (118 g, 49.9%)

### Synthesis Example 1-3 Synthesis of A-3

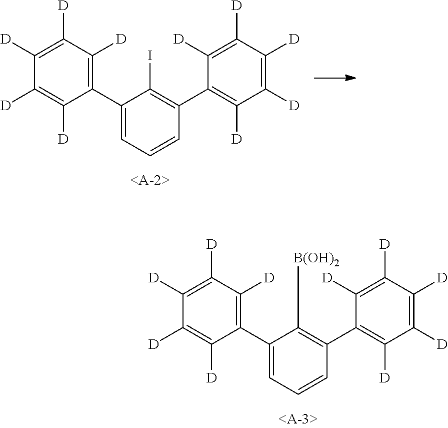 [SMILES: [2H]c1c([2H])c([2H])c(-c2cccc(-c3c([2H])c([2H])c([2H])c([2H])c3[2H])c2I)c([2H])c1[2H] ; [2H]c1c([2H])c([2H])c(-c2cccc(-c3c([2H])c([2H])c([2H])c([2H])c3[2H])c2B(O)O)c([2H])c1[2H]]

In a reactor, a mixture of <A-2> (118 g) and THF (1330 mL) was cooled to −78° C. and slowly added with drops of n-BuLi (241.6 g) while being stirred for 30 minutes. Thereafter, trimethyl borate (40.2 g) was dropwise added before the reaction mixture was elevated to room temperature. Drops of 2M HCl were added, followed by extraction with ethyl acetate. Crystallization in heptane and drying afforded <A-3>. (55 g, 60.1%)

### Synthesis Example 1-4 Synthesis of A-4

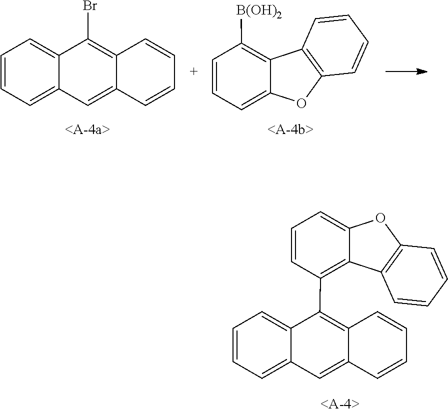

In a reactor, <A-4a> (100 g), <A-4b> (98.9 g), tetrakis(triphenylphosphine)palladium(0) (9 g), potassium carbonate (107.5 g), toluene (600 mL), ethanol (300 mL), and distilled water (300 mL) were stirred together under reflux and then reacted overnight. After the reaction was completed as monitored by TLC, the reaction mixture was cooled to room temperature. Extraction was conducted with distilled water and ethyl acetate. The ethyl acetate layer was concentrated and purified by column chromatography, followed by recrystallization to afford <A-4>. (117 g, 87.3%)

### Synthesis Example 1-5 Synthesis of A-5

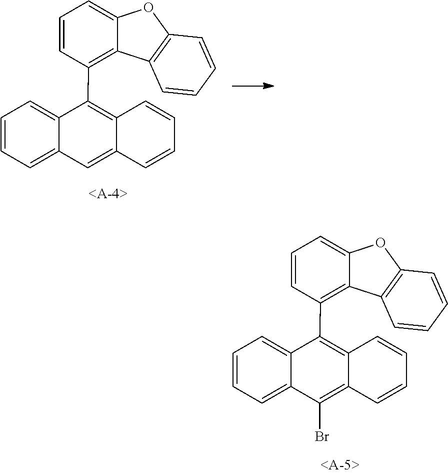 [SMILES: c1ccc2c(-c3cccc4oc5ccccc5c34)c3ccccc3cc2c1 ; Brc1c2ccccc2c(-c2cccc3oc4ccccc4c23)c2ccccc12]

In a reactor, drops of NBS (60.4 g) were slowly added to a mixture of <A-4> (117 g) and DMF (1170 mL), followed by stirring for 2 hours. Then, water was dropwise added.

The precipitate was filtered and dissolved in dichloromethane. After washing with distilled water, the dichloromethane layer was concentrated. Crystallization in heptane and subsequent drying afforded <A-5>. (128 g, 89%)

### Synthesis Example 1-6: Synthesis of [BH-1]

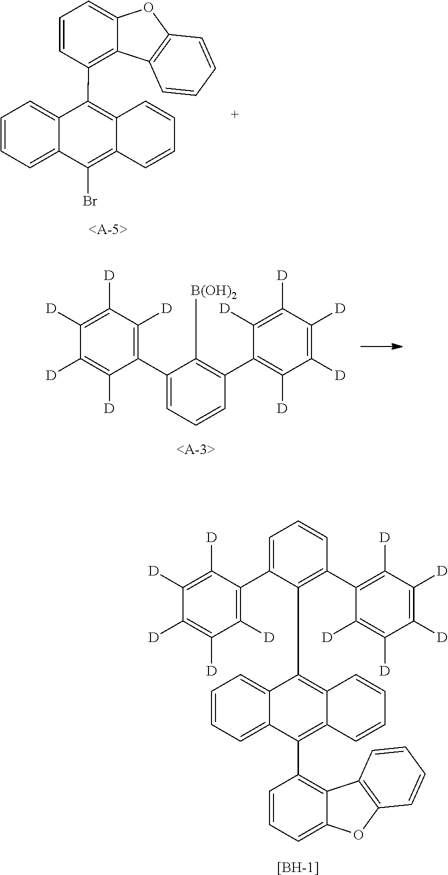

In a reactor, <A-5> (20 g), <A-3> (20.1 g), palladium (II) acetate (0.21 g), sodium tert-butoxide (13.6 g), BIDIME (0.62 g), and toluene (230 mL) were stirred together for 3 days under reflux. After completion of the reaction as monitored by TLC, the reaction mixture was cooled to room temperature and subjected to extraction with distilled water and ethyl acetate. The ethyl acetate layer was concentrated, and isolation by column chromatography and subsequent recrystallization afforded [BH-1]. (15.8 g, 57.4%)

MS (MALDI-TOF): m/z 582.28 [M⁺]

### Synthesis Example 2. Synthesis of [BH-2]

### Synthesis Example 2-1 Synthesis of B-1

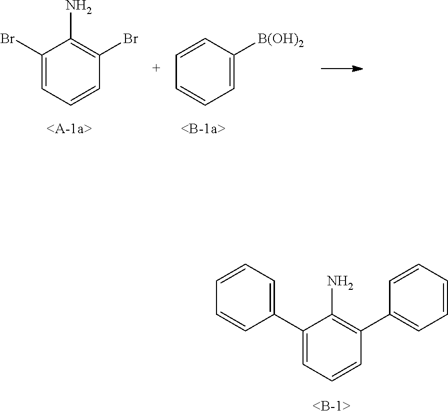 [SMILES: Nc1c(Br)cccc1Br ; OB(O)c1ccccc1 ; Nc1c(-c2ccccc2)cccc1-c1ccccc1]

The same procedure as in Synthesis Example 1-1 was carried out, with the exception of using <B-1a22 instead of <A-1b>, to afford <B-1> (yield 81.1%).

### Synthesis Example 2-2 Synthesis of B-2

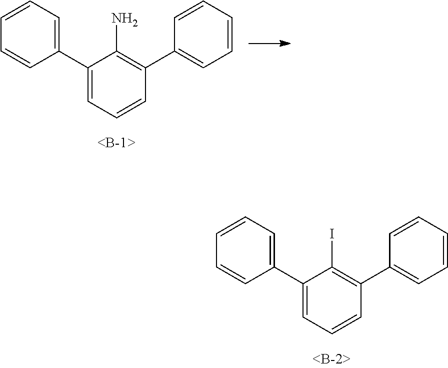 [SMILES: Nc1c(-c2ccccc2)cccc1-c1ccccc1 ; Ic1c(-c2ccccc2)cccc1-c1ccccc1]

The same procedure as in Synthesis Example 1-2 was carried out, with the exception of using <B-1> instead of <A-1>, to afford <B-2>. (yield 49.9%)

### Synthesis Example 2-3 Synthesis of B-3

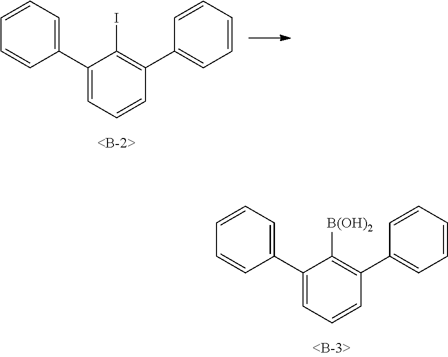 [SMILES: Ic1c(-c2ccccc2)cccc1-c1ccccc1 ; OB(O)c1c(-c2ccccc2)cccc1-c1ccccc1]

The same procedure as in Synthesis Example 1-3 was carried out, with the exception of using <B-2> instead of <A-2>, to afford <B-3>. (yield 60.1%)

### Synthesis Example 2-4 Synthesis of B-4

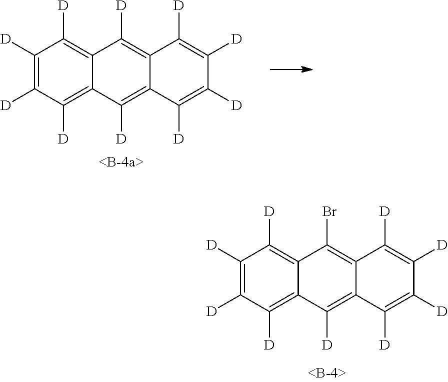 [SMILES: [2H]c1c([2H])c([2H])c2c([2H])c3c([2H])c([2H])c([2H])c([2H])c3c([2H])c2c1[2H] ; [2H]c1c([2H])c([2H])c2c(Br)c3c([2H])c([2H])c([2H])c([2H])c3c([2H])c2c1[2H]]

The same procedure as in Synthesis Example 1-5 was carried out, with the exception of using <B-4a> instead of <A-4>, to afford <B-4>. (yield 93.4%)

### Synthesis Example 2-5 Synthesis of B-5

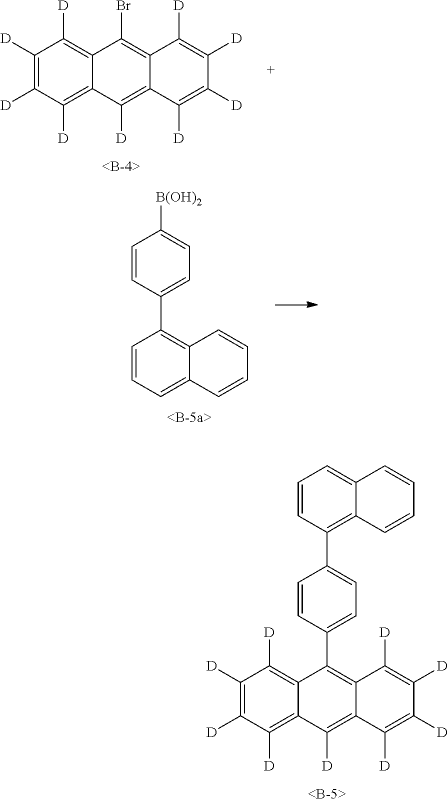 [SMILES: [2H]c1c([2H])c([2H])c2c(Br)c3c([2H])c([2H])c([2H])c([2H])c3c([2H])c2c1[2H] ; OB(O)c1ccc(-c2cccc3ccccc23)cc1 ; [2H]c1c([2H])c([2H])c2c(-c3ccc(-c4cccc5ccccc45)cc3)c3c([2H])c([2H])c([2H])c([2H])c3c([2H])c2c1[2H]]

The same procedure as in Synthesis Example 1-4 was carried out, with the exception of using <B-4> and <B-5a> instead of <A-4a> and <A-4b>, respectively, to afford <B-5>. (yield 88.5%)

### Synthesis Example 2-6 Synthesis of B-6

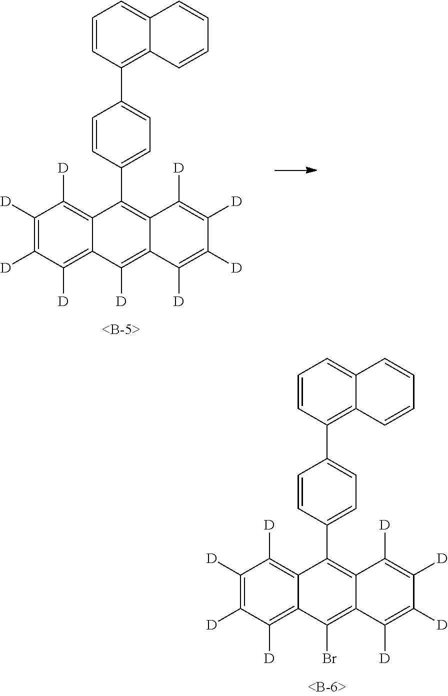 [SMILES: [2H]c1c([2H])c([2H])c2c(-c3ccc(-c4cccc5ccccc45)cc3)c3c([2H])c([2H])c([2H])c([2H])c3c([2H])c2c1[2H] ; [2H]c1c([2H])c([2H])c2c(-c3ccc(-c4cccc5ccccc45)cc3)c3c([2H])c([2H])c([2H])c([2H])c3c(Br)c2c1[2H]]

The same procedure as in Synthesis Example 1-5 was carried out, with the exception of using <B-5> instead of <A-4>, to afford <B-6>. (yield 85.4%)

### Synthesis Example 2-7: Synthesis of [BH-2]

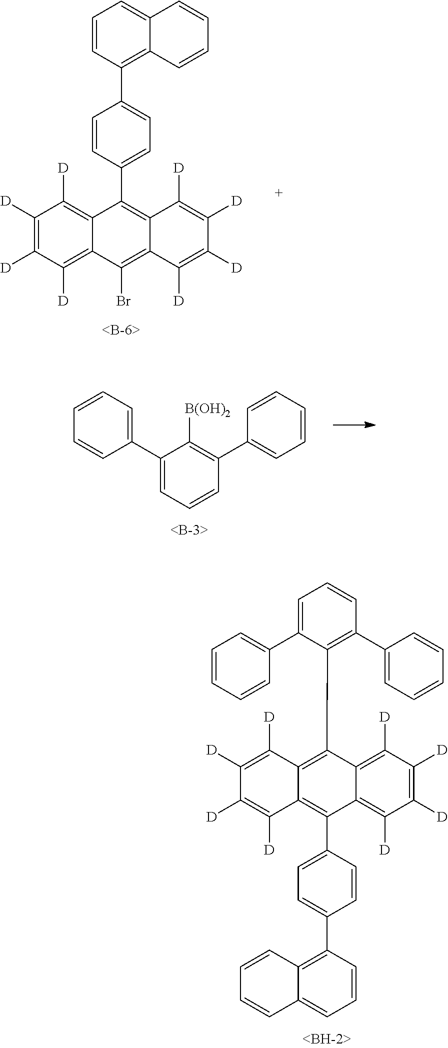 [SMILES: [2H]c1c([2H])c([2H])c2c(-c3ccc(-c4cccc5ccccc45)cc3)c3c([2H])c([2H])c([2H])c([2H])c3c(Br)c2c1[2H] ; OB(O)c1c(-c2ccccc2)cccc1-c1ccccc1 ; [2H]c1c([2H])c([2H])c2c(-c3c(-c4ccccc4)cccc3-c3ccccc3)c3c([2H])c([2H])c([2H])c([2H])c3c(-c3ccc(-c4cccc5ccccc45)cc3)c2c1[2H]]

The same procedure as in Synthesis Example 1-6 was carried out, with the exception of using <B-6> and <B-3> instead of <A-5> and <A-3>, respectively, to afford [BH-2](yield 58%)

MS (MALDI-TOF): m/z 616.30 [M⁺]

### Synthesis Example 3. Synthesis of [BH-3]

### Synthesis Example 3-1: Synthesis of C-1

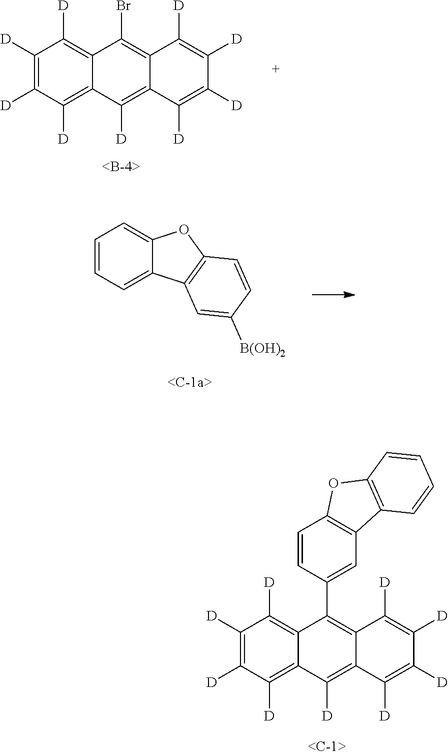 [SMILES: [2H]c1c([2H])c([2H])c2c(Br)c3c([2H])c([2H])c([2H])c([2H])c3c([2H])c2c1[2H] ; OB(O)c1ccc2oc3ccccc3c2c1 ; [2H]c1c([2H])c([2H])c2c(-c3ccc4oc5ccccc5c4c3)c3c([2H])c([2H])c([2H])c([2H])c3c([2H])c2c1[2H]]

The same procedure as in Synthesis Example 1-4 was carried out, with the exception of using <B-4> and <C-1a> instead of <A-4a> and <A-4b>, respectively, to afford <C-1>. (yield 79.8%)

### Synthesis Example 3-2 Synthesis of C-2

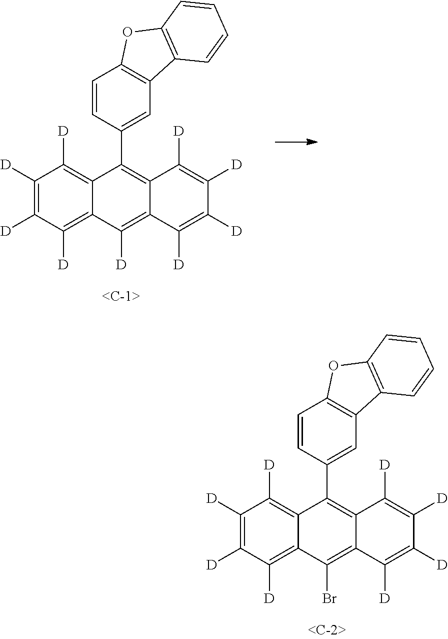 [SMILES: [2H]c1c([2H])c([2H])c2c(-c3ccc4oc5ccccc5c4c3)c3c([2H])c([2H])c([2H])c([2H])c3c([2H])c2c1[2H] ; [2H]c1c([2H])c([2H])c2c(-c3ccc4oc5ccccc5c4c3)c3c([2H])c([2H])c([2H])c([2H])c3c(Br)c2c1[2H]]

The same procedure as in Synthesis Example 1-5 was carried out, with the exception of using <C-1> instead of <A-4>, to afford <C-2>. (yield 83.7%)

### Synthesis Example 3-3 Synthesis of [BH-3]

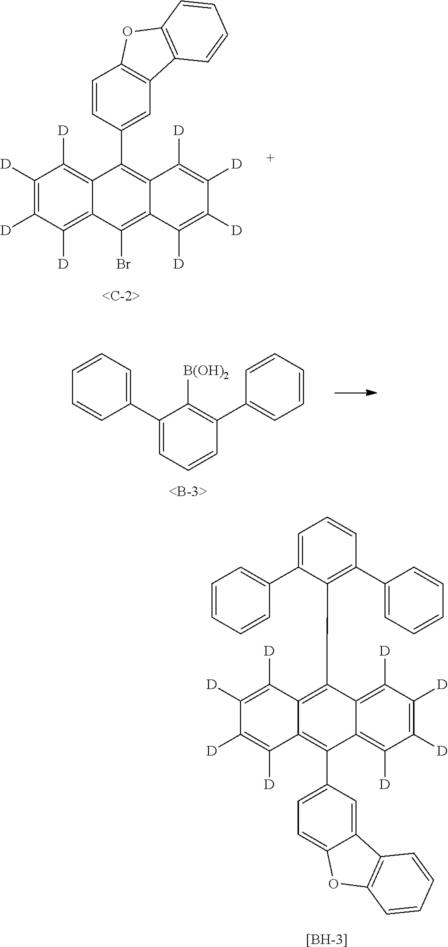

The same procedure as in Synthesis Example 1-6 was carried out, with the exception of using <C-2> and <B-3> instead of <A-5> and <A-3>, respectively, to afford [BH-3](yield 62%)

MS (MALDI-TOF): m/z 580.26 [M⁺]

### Synthesis Example 4. Synthesis of [BH-4]

### Synthesis Example 4-1 Synthesis of D-1

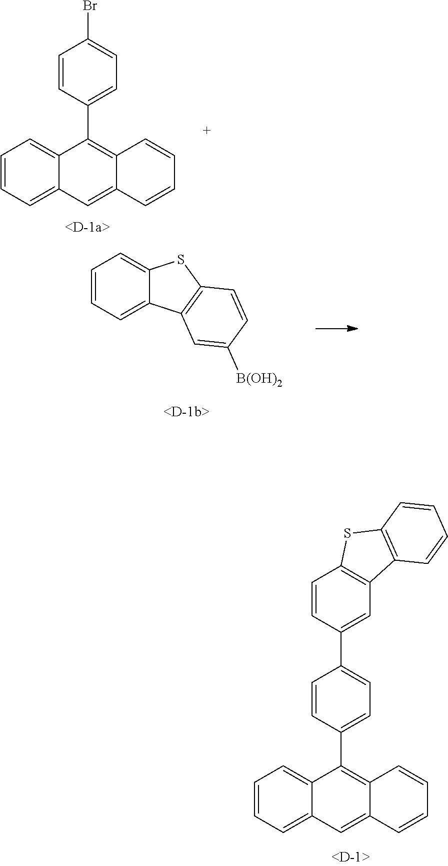 [SMILES: Brc1ccc(-c2c3ccccc3cc3ccccc23)cc1 ; OB(O)c1ccc2sc3ccccc3c2c1 ; c1ccc2c(-c3ccc(-c4ccc5sc6ccccc6c5c4)cc3)c3ccccc3cc2c1]

The same procedure as in Synthesis Example 1-4 was carried out, with the exception of using <D-1a> and <D-1b> instead of <A-4a> and <A-4b>, respectively, to afford <D-1>. (yield 85.4%)

### Synthesis Example 4-2 Synthesis of D-2

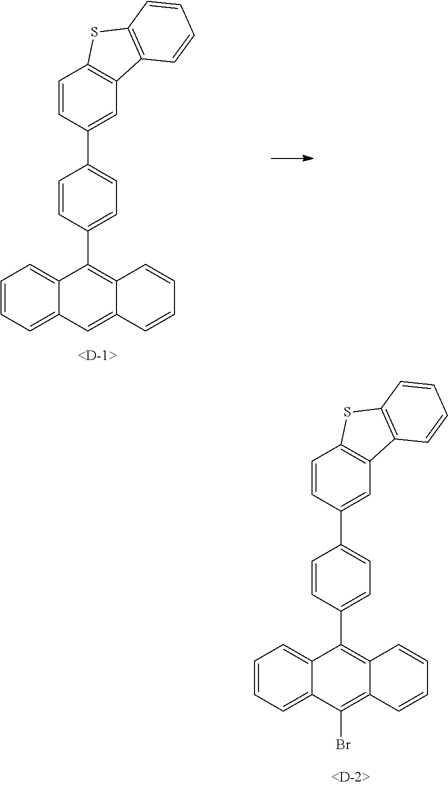 [SMILES: c1ccc2c(-c3ccc(-c4ccc5sc6ccccc6c5c4)cc3)c3ccccc3cc2c1 ; Brc1c2ccccc2c(-c2ccc(-c3ccc4sc5ccccc5c4c3)cc2)c2ccccc12]

The same procedure as in Synthesis Example 1-5 was carried out, with the exception of using <D-1> instead of <A-4>, to afford <D-2>. (yield 85.2%)

### Synthesis Example 4-3 Synthesis of [BH-4]

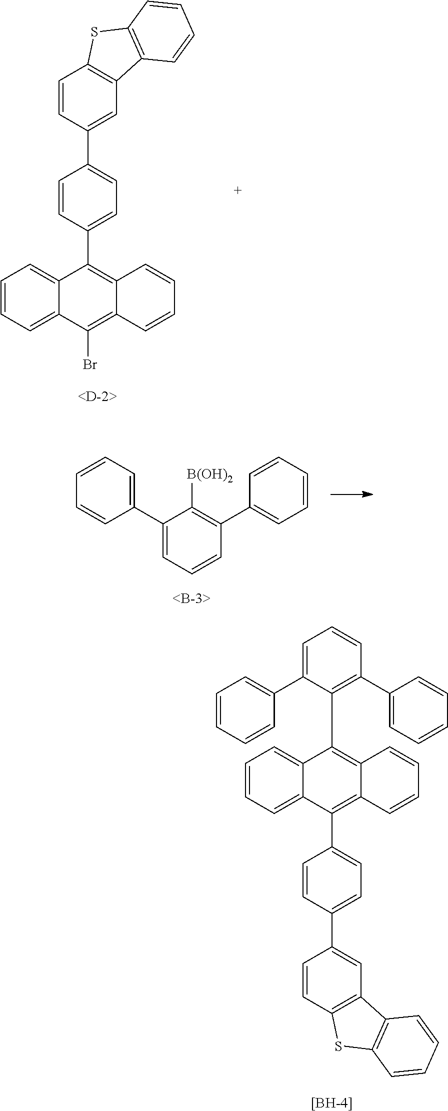

The same procedure as in Synthesis Example 1-6 was carried out, with the exception of using <D-2> and <B-3> instead of <A-5> and <A-3>, respectively, to afford [BH-4](yield 55.4%)

MS (MALDI-TOF): m/z 664.22 [M⁺]

### Synthesis Example 5. Synthesis of [BH-5]

### Synthesis Example 5-1: Synthesis of E-1

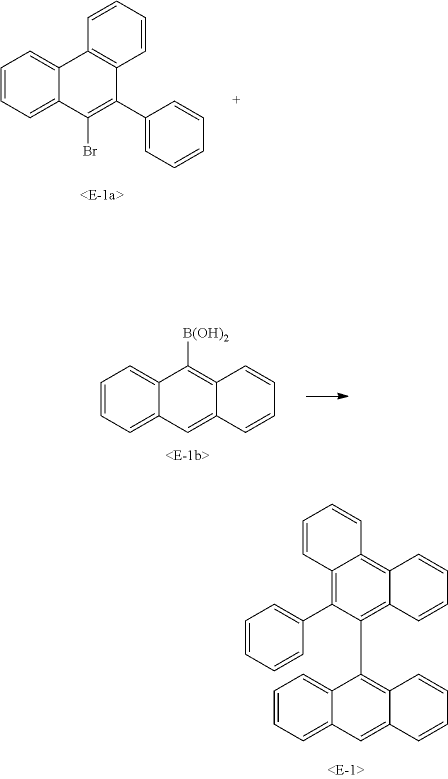

The same procedure as in Synthesis Example 1-4 was carried out, with the exception of using <E-1a> and <E-1b> instead of <A-4a> and <A-4b>, respectively, to afford <E-1>. (yield 81.9%)

### Synthesis Example 5-2: Synthesis of E-2

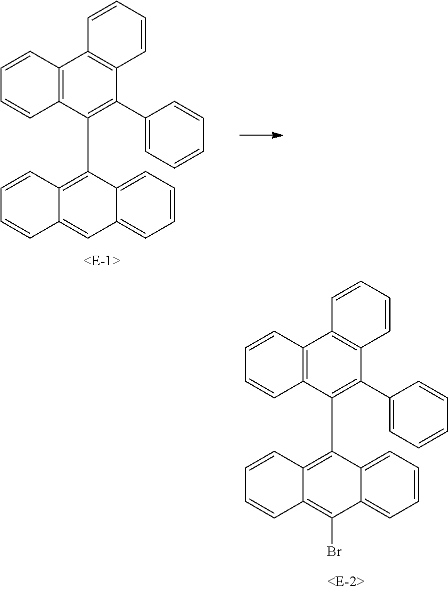

The same procedure as in Synthesis Example 1-5 was carried out, with the exception of using <E-1> instead of <A-4>, to afford <E-2>. (yield 82.3%)

### Synthesis Example 5-3 Synthesis of [BH-5]

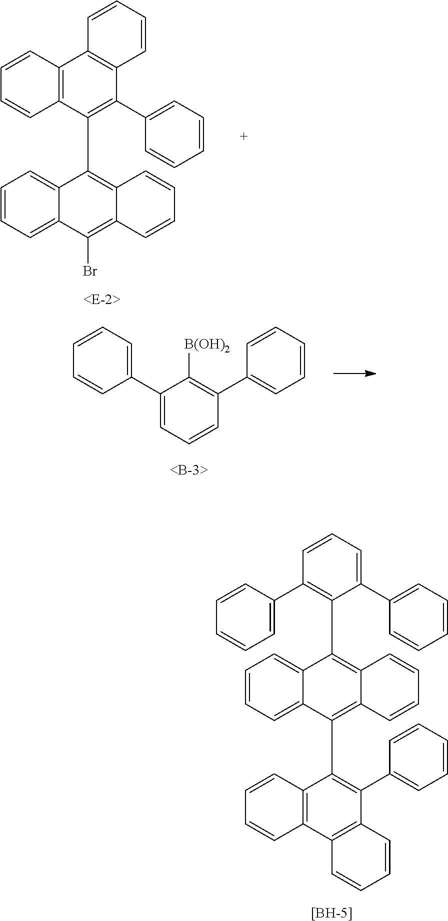

The same procedure as in Synthesis Example 1-6 was carried out, with the exception of using <E-2> and <B-3> instead of <A-5> and <A-3>, to afford [BH-5]. (yield 58.4%)

MS (MALDI-TOF): m/z 658.27 [M⁺]

### Synthesis Example 6. Synthesis of [BH-6]

### Synthesis Example 6-1: Synthesis of F-1

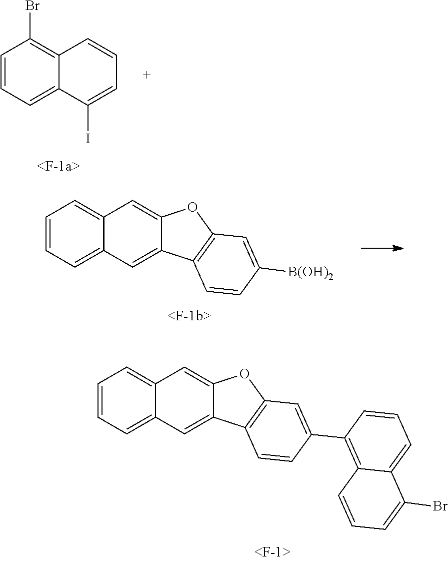 [SMILES: Brc1cccc2c(I)cccc12 ; OB(O)c1ccc2c(c1)oc1cc3ccccc3cc12 ; Brc1cccc2c(-c3ccc4c(c3)oc3cc5ccccc5cc34)cccc12]

The same procedure as in Synthesis Example 1-4 was carried out, with the exception of using <F-1a> and <F-1b> instead of <A-4a> and <A-4b>, respectively, to afford <F-1>. (yield 75.8%)

### Synthesis Example 6-2: Synthesis of F-2

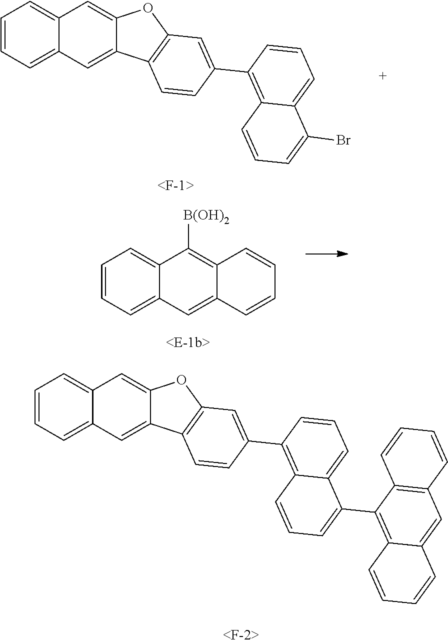 [SMILES: Brc1cccc2c(-c3ccc4c(c3)oc3cc5ccccc5cc34)cccc12 ; OB(O)c1c2ccccc2cc2ccccc12 ; c1ccc2cc3c(cc2c1)oc1cc(-c2cccc4c(-c5c6ccccc6cc6ccccc56)cccc24)ccc13]

The same procedure as in Synthesis Example 1-4 was carried out, with the exception of using <F-1> and <E-1b> instead of <A-4a> and <A-4b>, respectively, to afford <F-2>. (yield 82.4%)

### Synthesis Example 6-3 Synthesis of F-3

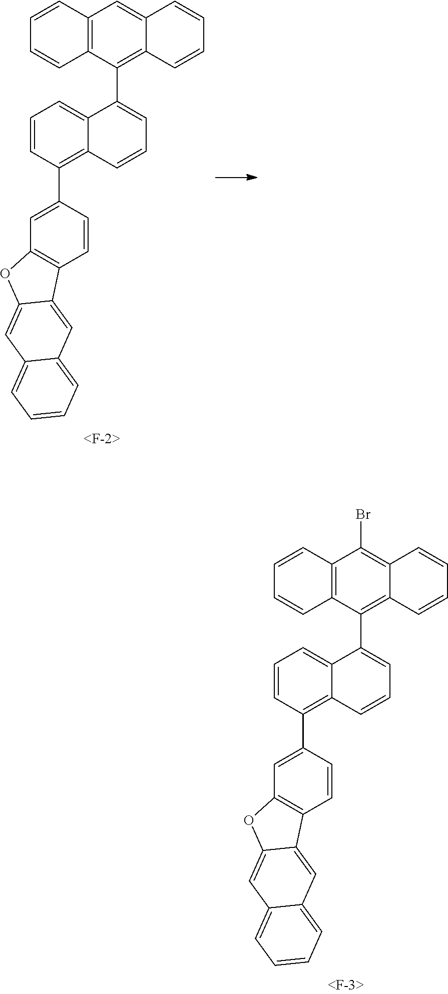 [SMILES: c1ccc2cc3c(cc2c1)oc1cc(-c2cccc4c(-c5c6ccccc6cc6ccccc56)cccc24)ccc13 ; Brc1c2ccccc2c(-c2cccc3c(-c4ccc5c(c4)oc4cc6ccccc6cc45)cccc23)c2ccccc12]

The same procedure as in Synthesis Example 1-5 was carried out, with the exception of using <F-2> instead of <A-4>, to afford <F-3>. (yield 78.9%)

### Synthesis Example 6-4 Synthesis of [BH-6]

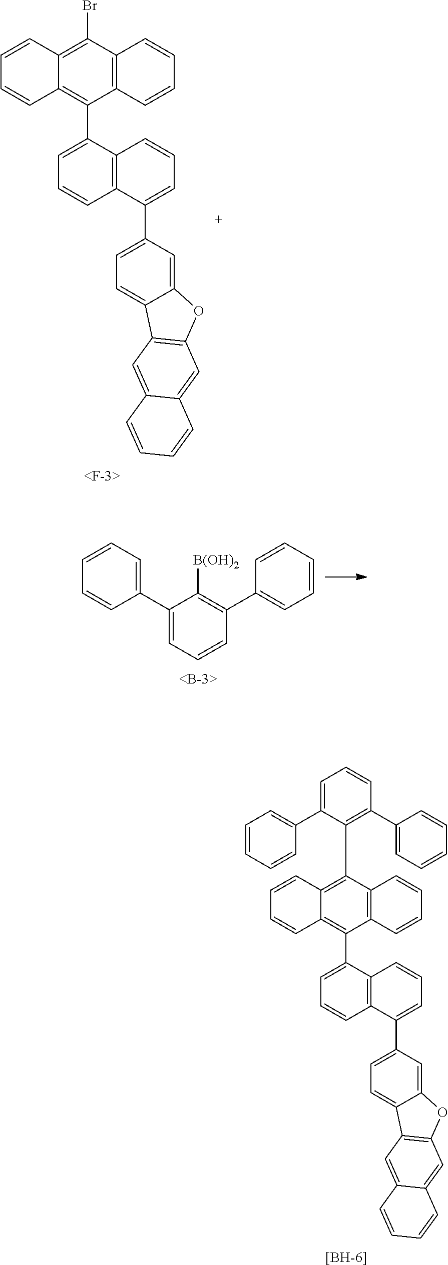

The same procedure as in Synthesis Example 1-6 was carried out, with the exception of using <F-3> and <B-3> instead of <A-5> and <A-3>, respectively, to afford [BH-6](yield 55.2%)

MS (MALDI-TOF): m/z 748.28 [M⁺]

### Synthesis Example 7. Synthesis of [BH-7]

### Synthesis Example 7-1 Synthesis of G-1

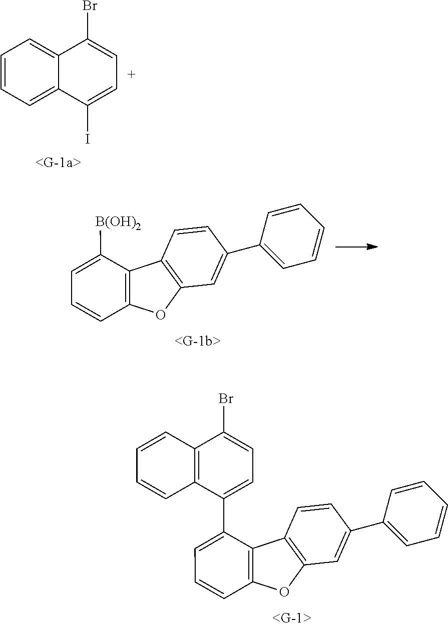 [SMILES: Brc1ccc(I)c2ccccc12 ; OB(O)c1cccc2oc3cc(-c4ccccc4)ccc3c12 ; Brc1ccc(-c2cccc3oc4cc(-c5ccccc5)ccc4c23)c2ccccc12]

The same procedure as in Synthesis Example 1-4 was carried out, with the exception of using <G-1a> and <G-1b> instead of <A-4a> and <A-4b>, respectively, to afford <G-1>. (yield 77.4%)

### Synthesis Example 7-2 Synthesis of G-2

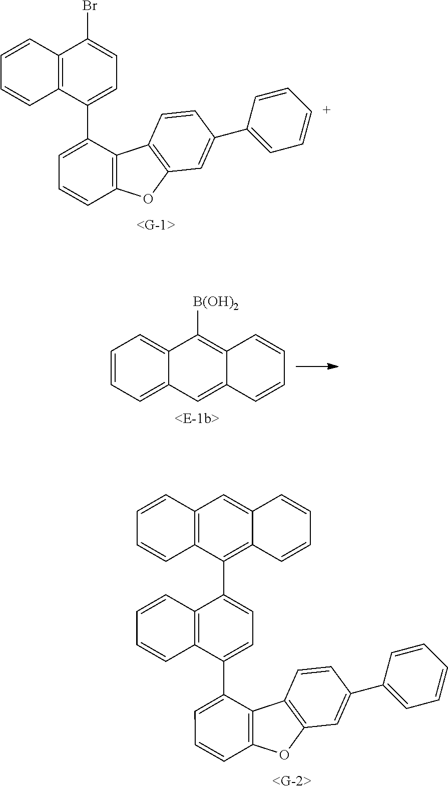 [SMILES: Brc1ccc(-c2cccc3oc4cc(C5=CC=[C+]C=C5)ccc4c23)c2ccccc12 ; OB(O)c1c2ccccc2cc2ccccc12 ; c1ccc(-c2ccc3c(c2)oc2cccc(-c4ccc(-c5c6ccccc6cc6ccccc56)c5ccccc45)c23)cc1]

The same procedure as in Synthesis Example 1-4 was carried out, with the exception of using <G-1> and <E-1b> instead of <A-4a> and <A-4b>, respectively, to afford <G-2>. (yield 87.5%)

### Synthesis Example 7-3 Synthesis of G-3

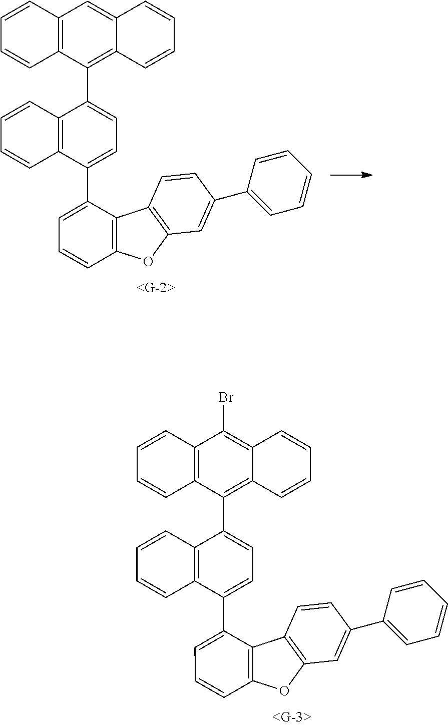 [SMILES: c1ccc(-c2ccc3c(c2)oc2cccc(-c4ccc(-c5c6ccccc6cc6ccccc56)c5ccccc45)c23)cc1 ; Brc1c2ccccc2c(-c2ccc(-c3cccc4oc5cc(-c6ccccc6)ccc5c34)c3ccccc23)c2ccccc12]

The same procedure as in Synthesis Example 1-5 was carried out, with the exception of using <G-2> instead of <A-4>, to afford <G-3>. (yield 76.3%)

### Synthesis Example 7-4 Synthesis of [BH-7]

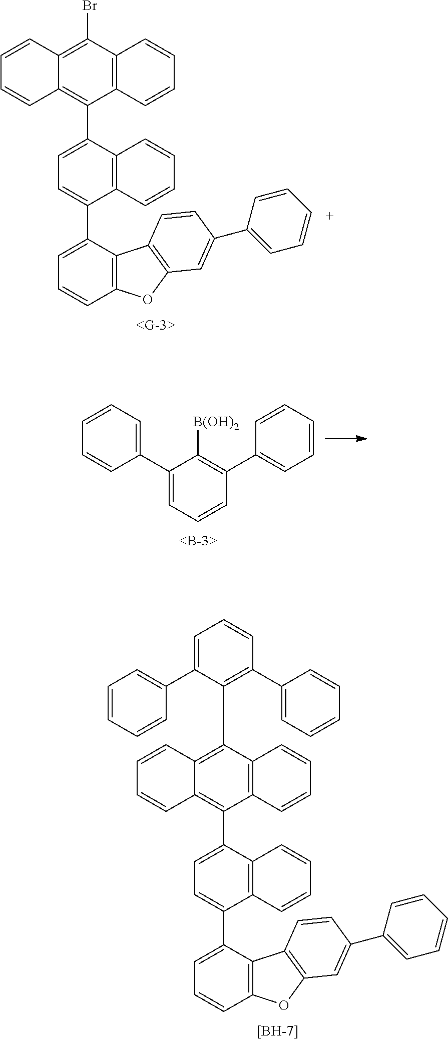

The same procedure as in Synthesis Example 1-6 was carried out, with the exception of using <G-3> and <B-3> instead of <A-5> and <A-3>, respectively, to afford [BH-7](yield 57.7%)

MS (MALDI-TOF): m/z 774.29 [M⁺]

### Synthesis Example 8. Synthesis of [BH-8]

### Synthesis Example 8-1 Synthesis of H-1

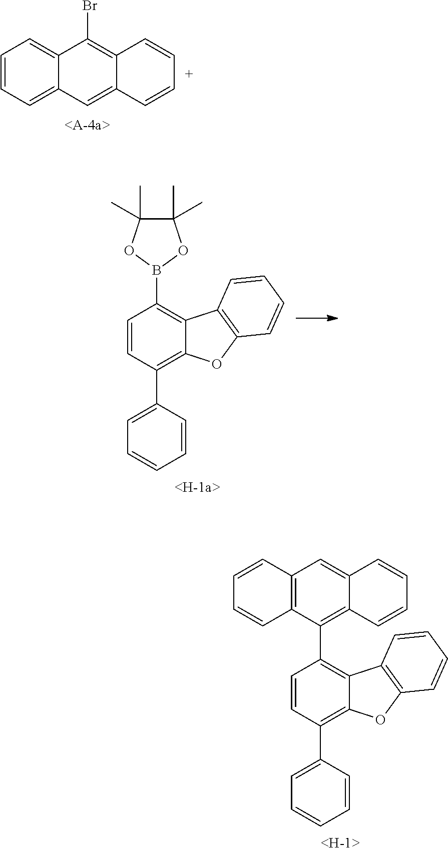 [SMILES: Brc1c2ccccc2cc2ccccc12 ; CC1(C)OB(c2ccc(-c3ccccc3)c3oc4ccccc4c23)OC1(C)C ; c1ccc(-c2ccc(-c3c4ccccc4cc4ccccc34)c3c2oc2ccccc23)cc1]

The same procedure as in Synthesis Example 1-4 was carried out, with the exception of using <H-1a> instead of <A-4b>, to afford <H-1>. (yield 83.5%)

### Synthesis Example 8-2 Synthesis of H-2

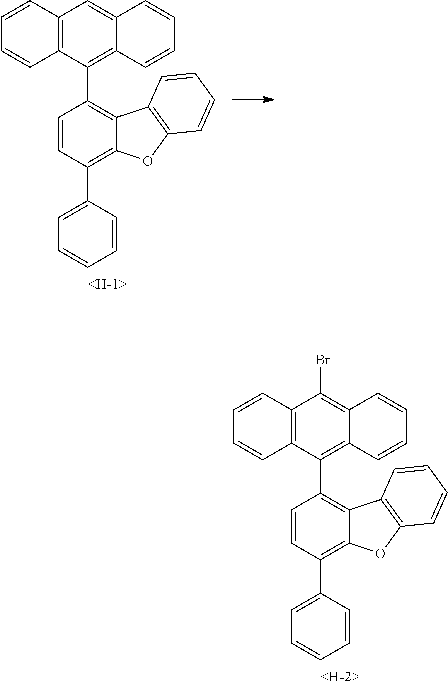 [SMILES: c1ccc(-c2ccc(-c3c4ccccc4cc4ccccc34)c3c2oc2ccccc23)cc1 ; Brc1c2ccccc2c(-c2ccc(-c3ccccc3)c3oc4ccccc4c23)c2ccccc12]

The same procedure as in Synthesis Example 1-5 was carried out, with the exception of using <H-1> instead of <A-4>, to afford <H-2>. (yield 85.3%)

### Synthesis Example 8-3 Synthesis of [BH-8]

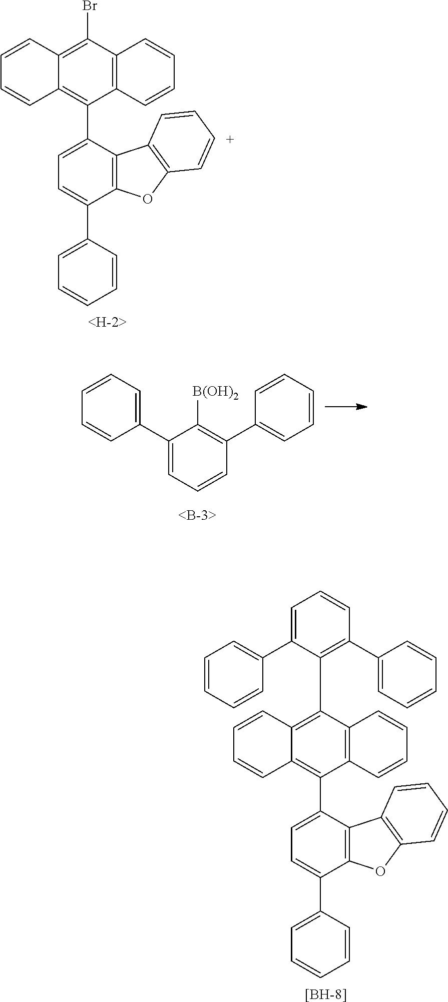

The same procedure as in Synthesis Example 1-6 was carried out, with the exception of using <H-2> and <B-3> instead of <A-5> and <A-3>, respectively, to afford [BH-8](yield 52%)

MS (MALDI-TOF): m/z 648.25 [M⁺]

### Synthesis Example 9. Synthesis of [BH-9]

### Synthesis Example 9-1: Synthesis of I-1

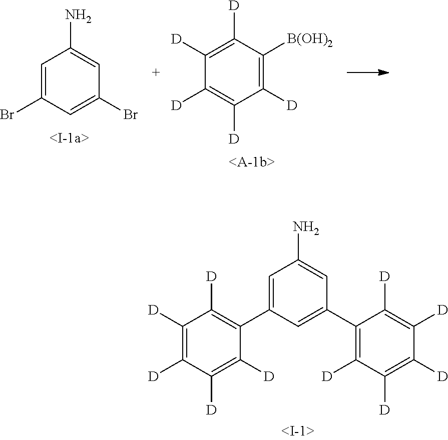 [SMILES: Nc1cc(Br)cc(Br)c1 ; [2H]c1c([2H])c([2H])c(B(O)O)c([2H])c1[2H] ; [2H]c1c([2H])c([2H])c(-c2cc(N)cc(-c3c([2H])c([2H])c([2H])c([2H])c3[2H])c2)c([2H])c1[2H]]

The same procedure as in Synthesis Example 1-1 was carried out, with the exception of using <I-1a> instead of <A-1a>, to afford <I-1>. (yield 79.2%)

### Synthesis Example 9-2 Synthesis of I-2

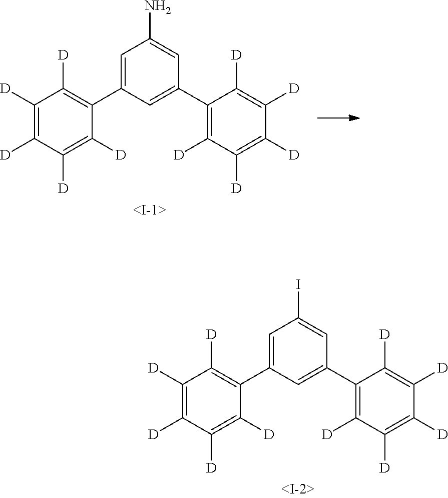 [SMILES: [2H]c1c([2H])c([2H])c(-c2cc(N)cc(-c3c([2H])c([2H])c([2H])c([2H])c3[2H])c2)c([2H])c1[2H] ; [2H]c1c([2H])c([2H])c(-c2cc(I)cc(-c3c([2H])c([2H])c([2H])c([2H])c3[2H])c2)c([2H])c1[2H]]

The same procedure as in Synthesis Example 1-2 was carried out, with the exception of using <I-1> instead of <A-1>, to afford <I-2>. (yield 50.2%)

### Synthesis Example 9-3 Synthesis of I-3

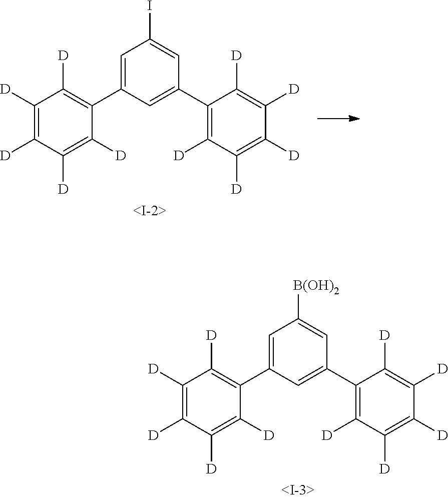 [SMILES: [2H]c1c([2H])c([2H])c(-c2cc(I)cc(-c3c([2H])c([2H])c([2H])c([2H])c3[2H])c2)c([2H])c1[2H] ; [2H]c1c([2H])c([2H])c(-c2cc(B(O)O)cc(-c3c([2H])c([2H])c([2H])c([2H])c3[2H])c2)c([2H])c1[2H]]

The same procedure as in Synthesis Example 1-3 was carried out, with the exception of using <I-2> instead of <A-2>, to afford <I-3>. (yield 58.7%)

### Synthesis Example 9-4 Synthesis of I-4

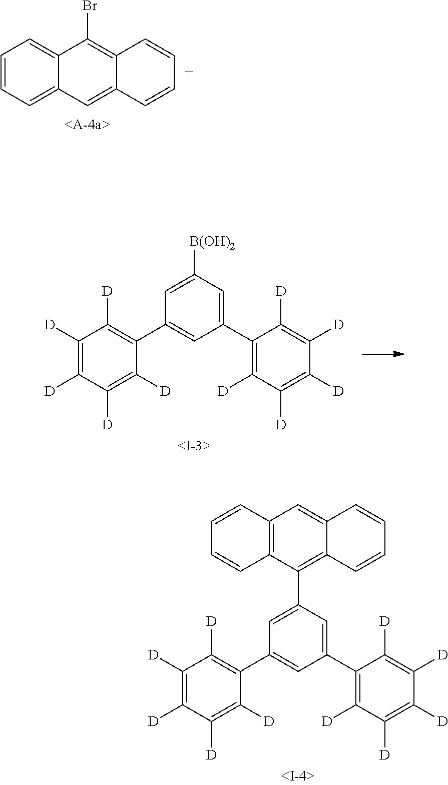 [SMILES: Brc1c2ccccc2cc2ccccc12 ; [2H]c1c([2H])c([2H])c(-c2cc(B(O)O)cc(-c3c([2H])c([2H])c([2H])c([2H])c3[2H])c2)c([2H])c1[2H] ; [2H]c1c([2H])c([2H])c(-c2cc(-c3c([2H])c([2H])c([2H])c([2H])c3[2H])cc(-c3c4ccccc4cc4ccccc34)c2)c([2H])c1[2H]]

The same procedure as in Synthesis Example 1-4 was carried out, with the exception of using <I-3> instead of <A-4b>, to afford <I-4>. (yield 77.4%)

### Synthesis Example 9-5 Synthesis of I-5

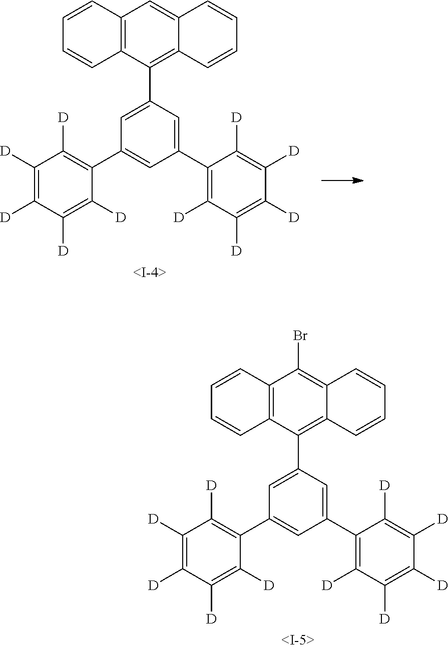 [SMILES: [2H]c1c([2H])c([2H])c(-c2cc(-c3c([2H])c([2H])c([2H])c([2H])c3[2H])cc(-c3c4ccccc4cc4ccccc34)c2)c([2H])c1[2H] ; [2H]c1c([2H])c([2H])c(-c2cc(-c3c([2H])c([2H])c([2H])c([2H])c3[2H])cc(-c3c4ccccc4c(Br)c4ccccc34)c2)c([2H])c1[2H]]

The same procedure as in Synthesis Example 1-5 was carried out, with the exception of using <I-4> instead of <A-4>, to afford <I-5>. (yield 84.9%)

### Synthesis Example 9-6 Synthesis of [BH-9]

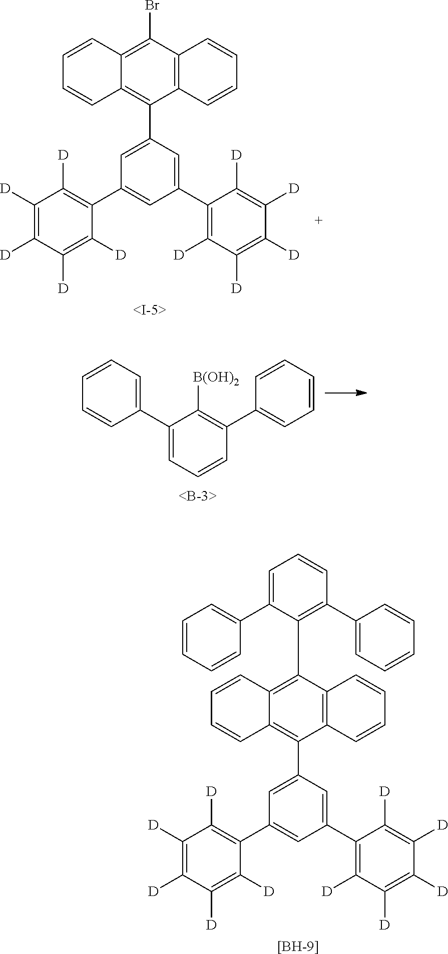

The same procedure as in Synthesis Example 1-6 was carried out, with the exception of using <I-5> and <B-3> instead of <A-5> and <A-3>, respectively, to afford [BH-9](yield 56.9%)

MS (MALDI-TOF): m/z 644.33 [M⁺]

### Synthesis Example 10. Synthesis of [BH-10]

### Synthesis Example 10-1 Synthesis of J-1

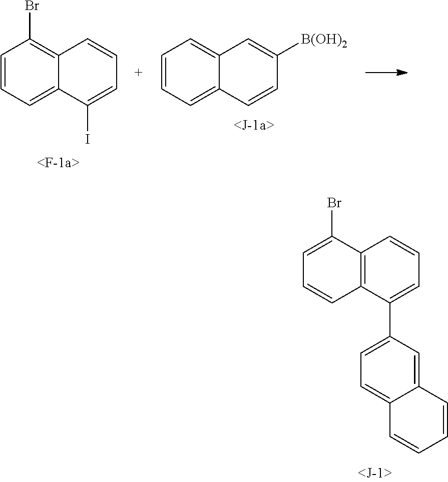

The same procedure as in Synthesis Example 1-4 was carried out, with the exception of using <F-1a> and <J-1a> instead of <A-4a> and <A-4b>, respectively, to afford <J-1>. (yield 79.2%)

### Synthesis Example 10-2 Synthesis of J-2

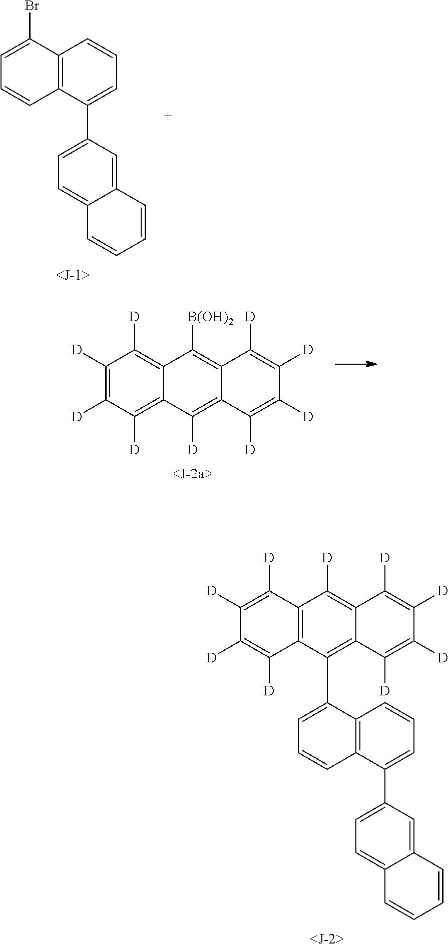 [SMILES: Brc1cccc2c(-c3ccc4ccccc4c3)cccc12 ; [2H]c1c([2H])c([2H])c2c(B(O)O)c3c([2H])c([2H])c([2H])c([2H])c3c([2H])c2c1[2H] ; [2H]c1c([2H])c([2H])c2c(-c3cccc4c(-c5ccc6ccccc6c5)cccc34)c3c([2H])c([2H])c([2H])c([2H])c3c([2H])c2c1[2H]]

The same procedure as in Synthesis Example 1-4 was carried out, with the exception of using <J-1> and <J-2a> instead of <A-4a> and <A-4b>, respectively, to afford <J-2>. (yield 84.1%)

### Synthesis Example 10-3 Synthesis of J-3

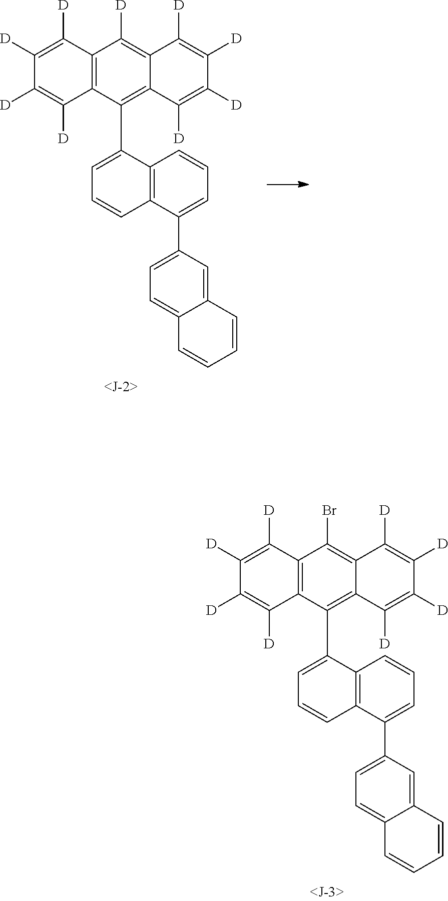 [SMILES: [2H]c1c([2H])c([2H])c2c(-c3cccc4c(-c5ccc6ccccc6c5)cccc34)c3c([2H])c([2H])c([2H])c([2H])c3c([2H])c2c1[2H] ; [2H]c1c([2H])c([2H])c2c(-c3cccc4c(-c5ccc6ccccc6c5)cccc34)c3c([2H])c([2H])c([2H])c([2H])c3c(Br)c2c1[2H]]

The same procedure as in Synthesis Example 1-5 was carried out, with the exception of using <J-2> instead of <A-4>, to afford <J-3>. (yield 84.3%)

### Synthesis Example 10-4 Synthesis of [BH-10]

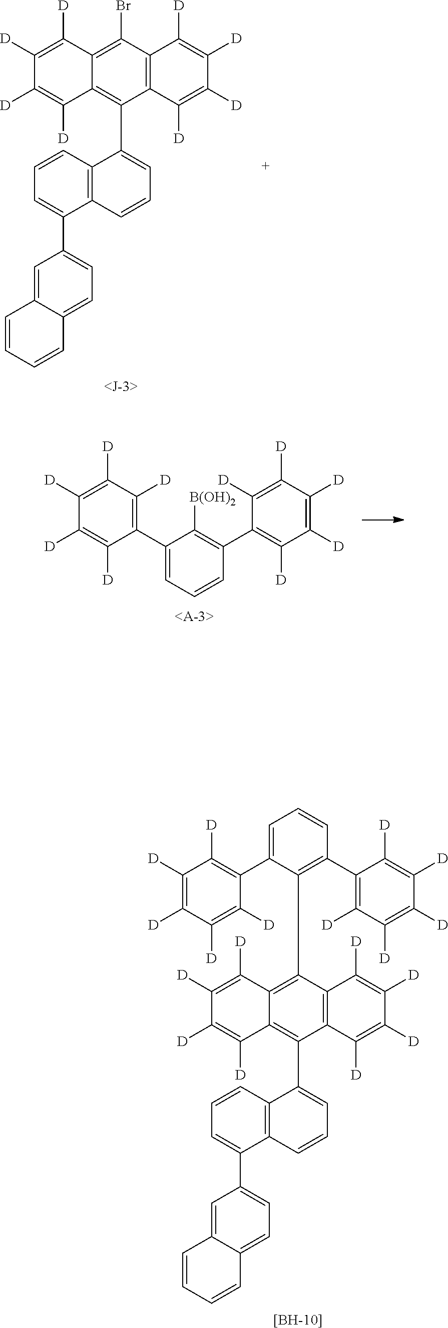

The same procedure as in Synthesis Example 1-6 was carried out, with the exception of using <J-3> instead of <A-5>, to afford [BH-10]. (yield 57%)

MS (MALDI-TOF): m/z 676.38 [M⁺]

### Synthesis of Dopant Materials

BD-1 and BD-2 were synthesized according to the method as described in Korean Patent No. 10-2148296, and concrete synthesis processes are illustrated in the following.

### Synthesis Example 11. Synthesis of [BD-3]

### Synthesis Example 11-1 Synthesis of K-1

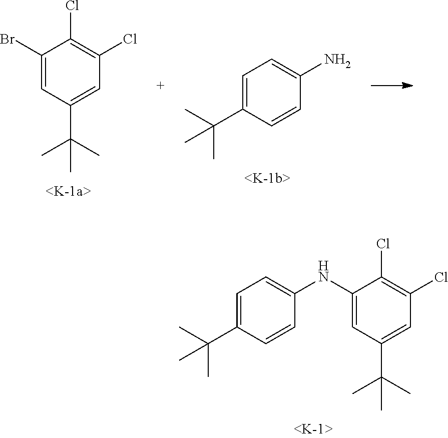 [SMILES: CC(C)(C)c1cc(Cl)c(Cl)c(Br)c1 ; CC(C)(C)c1ccc(N)cc1 ; CC(C)(C)c1ccc(Nc2cc(C(C)(C)C)cc(Cl)c2Cl)cc1]

In a 1-L reactor, <K-1a> (30 g), <K-1b> (20.6 g), palladium acetate (0.7 g), sodium tert-butoxide (20.5 g), bis(diphenylphosphino)-1,1′-binaphthyl (2 g), and toluene (300 mL) were stirred together for 24 hours under reflux. After completion of the reaction, filtration, concentration, and isolation by column chromatography afforded <K-1>. (31.5 g, 84.5%)

### Synthesis Example 11-2: Synthesis of K-2

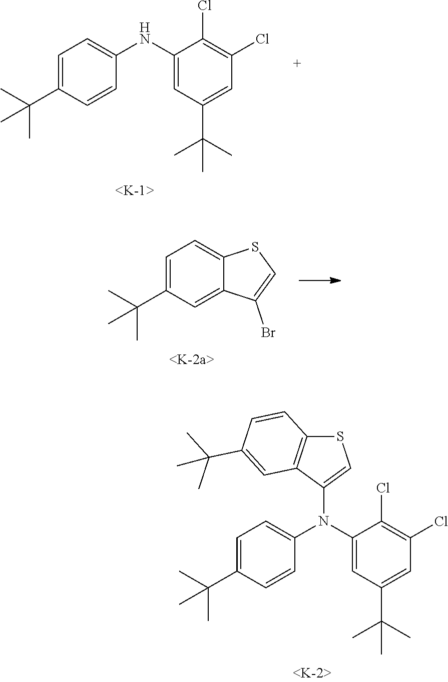 [SMILES: CC(C)(C)c1ccc(Nc2cc(C(C)(C)C)cc(Cl)c2Cl)cc1 ; CC(C)(C)c1ccc2scc(Br)c2c1 ; CC(C)(C)c1ccc(N(c2cc(C(C)(C)C)cc(Cl)c2Cl)c2csc3ccc(C(C)(C)C)cc23)cc1]

In a 250-mL reactor, <K-1> (20 g), <K-2a> (14.6 g), palladium acetate (0.32 g), sodium tert-butoxide (11 g), tri-tert-butylphosphine (0.46 g), and toluene (200 mL) were stirred together under reflux. After completion of the reaction, concentration and column chromatographic isolation afforded <K-2>. (22.7 g, 73.8%)

### Synthesis Example 11-3 Synthesis of K-3

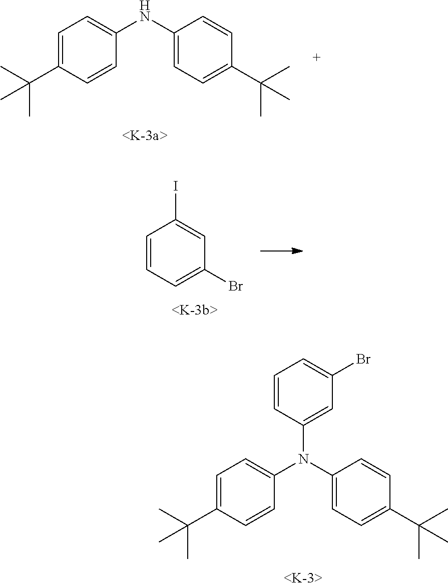

The same procedure as in Synthesis Example 11-2 was carried out, with the exception of using <<K-3a> and <K-3b> instead of and <K-2a>, respectively, to afford <K-3>. (yield 79.2%)

### Synthesis Example 11-4: Synthesis of K-4

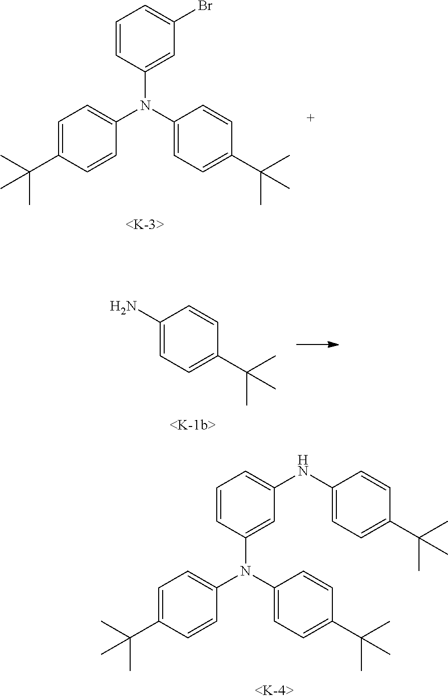

The same procedure as in Synthesis Example 11-2 was carried out, with the exception of using <<−3> and <K-1b> instead of and <K-2a>, respectively, to afford <K-4>. (yield 72.3%)

### Synthesis Example 11-5 Synthesis of K-5

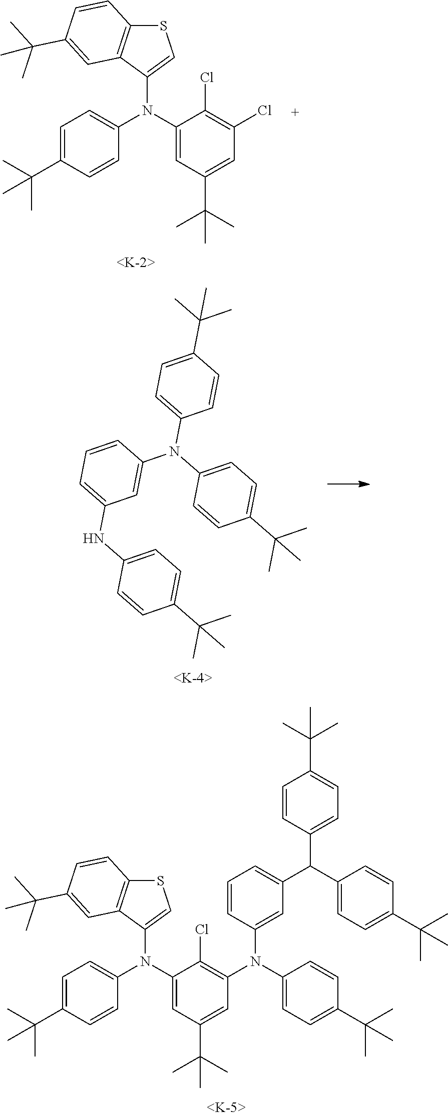

The same procedure as in Synthesis Example 11-2 was carried out, with the exception of using <K-2> and <K-4> instead of <K-1> and <K-2a>, respectively, to afford <K-5>. (yield 66.7%)

### Synthesis Example 11-6: Synthesis of [BD-3]

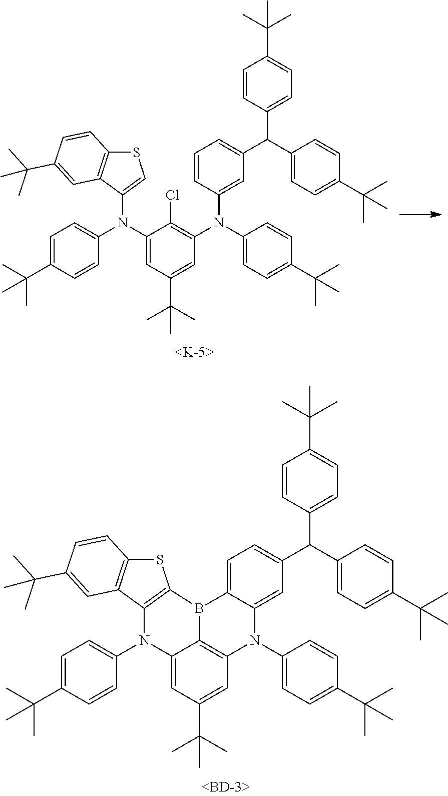

In a 250-ml reactor, a mixture of <K-5> (20 g) and tert-butyl benzene was added at −78° C. with drops of tert-butyl lithium (35 mL) and then stirred at 60° C. for 3 hours. Then, nitrogen was blown into the reactor to remove pentane. At −78° C., boron tribromide (4 mL) was dropwise added, followed by stirring at room temperature for 1 hour. At 0° C., N,N-diisopropylethyl amine (5.1 g) was dropwise added before stirring at 120° C. for 2 hours. After completion of the reaction, an aqueous sodium acetate solution was added and stirred. Extraction was conducted with ethyl acetate and the organic layer was concentrated, followed by isolation through column chromatography to afford [BD-1]. (3 g, 15.4%)

MS (MALDI-TOF): m/z 979.60 [M⁺]

### Synthesis Example 12. Synthesis of [BD-13]

### Synthesis Example 12-1: Synthesis of [BD-13]

The same procedure as in Synthesis Example 11 was carried out, with the exception of using 1-bromo-2,3-dichlorobenzene instead of <K-1a> in Synthesis Example 11-1 and dibenzo[b,d]furan-4-amine instead of <K-1b> in Synthesis Example 11-4, to afford [BD-13]. (yield 13.2%)

MS (MALDI-TOF): m/z 957.49 [M⁺]

### Synthesis Example Synthesis of 13. Synthesis of [BD-26]

### Synthesis Example 13-1: Synthesis of L-1

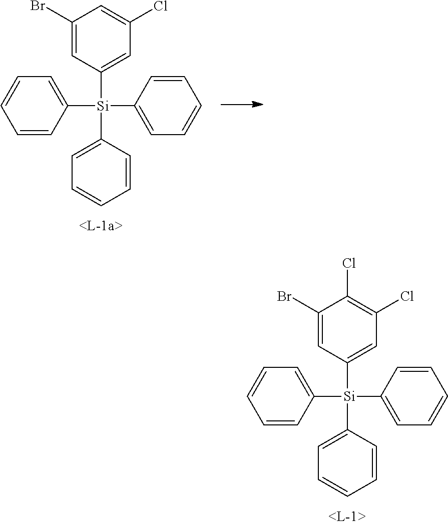 [SMILES: Clc1cc(Br)cc([Si](c2ccccc2)(c2ccccc2)c2ccccc2)c1 ; Clc1cc([Si](c2ccccc2)(c2ccccc2)c2ccccc2)cc(Br)c1Cl]

In a reactor, a mixture of <L-1a> (50 g) and tetrahydrofuran (50 mL) was dropwise added with 2.0 M lithium diisopropylamide solution (140 mL) at −78° C. and stirred for 3 hours at the same temperature. After slow addition of hexachloroethane, the reaction mixture was elevated to room temperature and stirred for 16 hours. Ethyl acetate and water were added and the organic layer thus formed was purified by silica gel chromatography to afford <L-1> (42.5 g, 78.9%)

### Synthesis Example 13-2 Synthesis of L-2

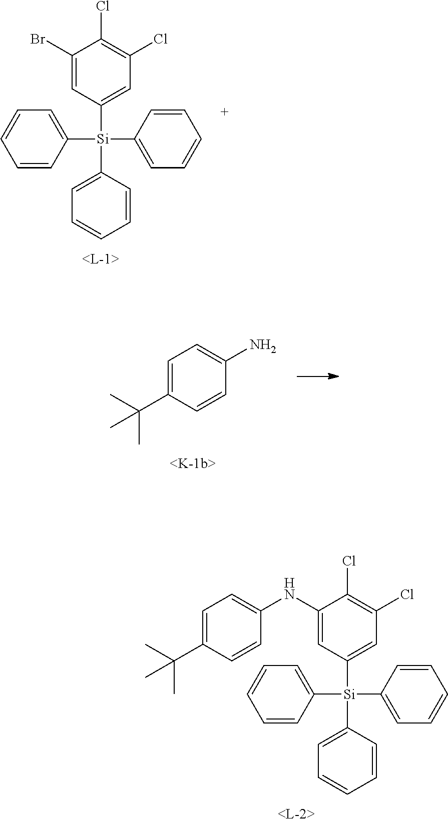 [SMILES: Clc1cc([Si](c2ccccc2)(c2ccccc2)c2ccccc2)cc(Br)c1Cl ; CC(C)(C)c1ccc(N)cc1 ; CC(C)(C)c1ccc(Nc2cc([Si](c3ccccc3)(c3ccccc3)c3ccccc3)cc(Cl)c2Cl)cc1]

The same procedure as in Synthesis Example 11-1 was carried out, with the exception of using <L-1> instead of <K-1a>, to afford <L-2>. (yield 56%)

### Synthesis Example 13-3 Synthesis of L-3

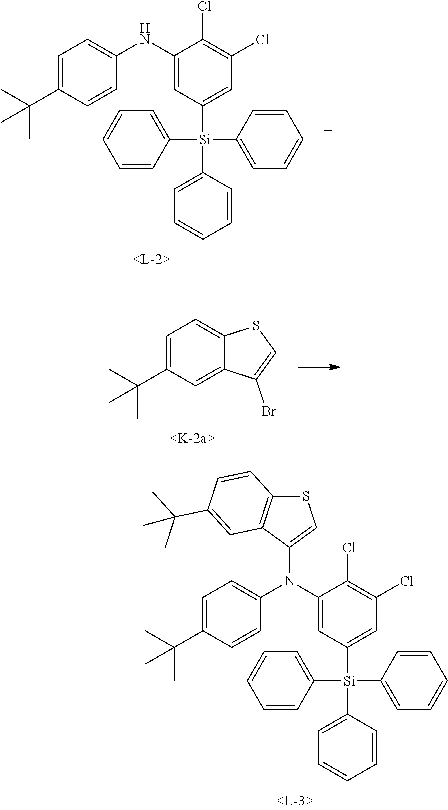 [SMILES: CC(C)(C)c1ccc(Nc2cc([Si](c3ccccc3)(c3ccccc3)c3ccccc3)cc(Cl)c2Cl)cc1 ; CC(C)(C)c1ccc2scc(Br)c2c1 ; CC(C)(C)c1ccc(N(c2cc([Si](c3ccccc3)(c3ccccc3)c3ccccc3)cc(Cl)c2Cl)c2csc3ccc(C(C)(C)C)cc23)cc1]

The same procedure as in Synthesis Example 11-2 was carried out, with the exception of using <L-2> instead of <K-1>, to afford <L-3>. (yield 84.3%)

### Synthesis Example 13-4 Synthesis of L-4

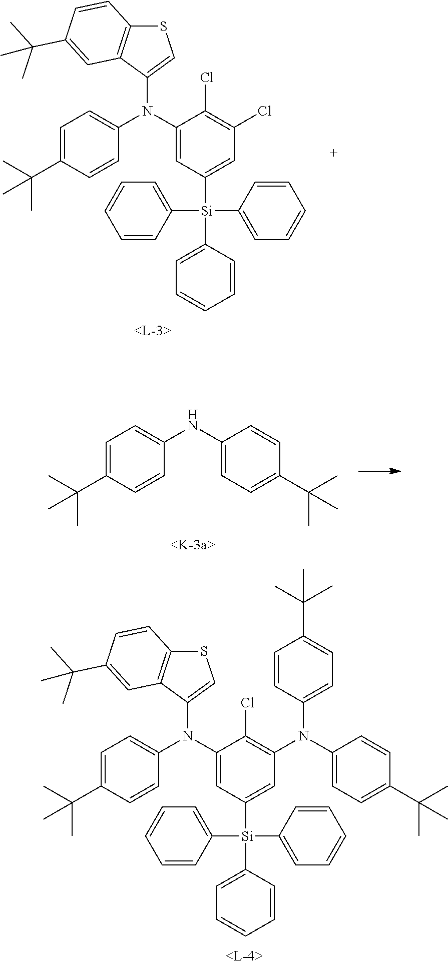

The same procedure as in Synthesis Example 11-3 was carried out, with the exception of using <L-3> instead of <K-3b>, to afford <L-4>. (yield 79.7%)

### Synthesis Example 13-5 Synthesis of [BD-26]

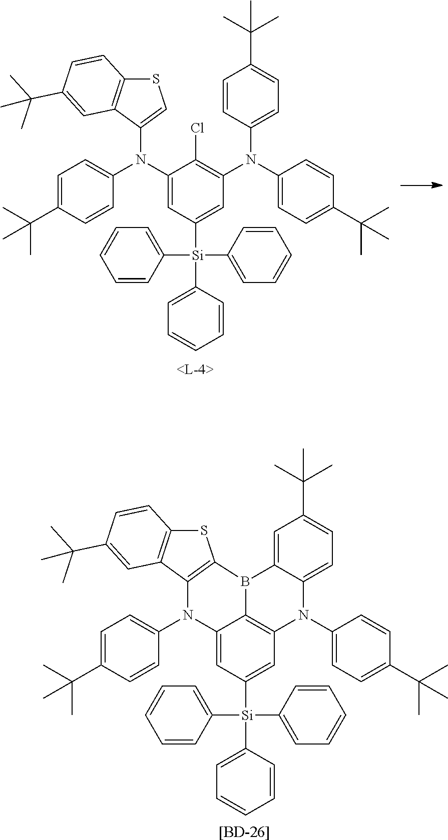

The same procedure as in Synthesis Example 11-4 was carried out, with the exception of using <L-4> instead of <K-3>, to afford [BD-26]. (yield 13.7%)

MS (MALDI-TOF) m/z 958.49 [M⁺]

### Example 1 TO 17 Fabrication of Organic Light-Emitting Diodes

An ITO glass substrate was patterned to have a translucent area of 2 mm×2 mm and cleansed. The ITO glass was mounted in a vacuum chamber that was then set to have a base pressure of 1×10⁻⁷ torr. The compound of the following structural formula [2-TNATA]was deposited to form a film (400 Å) as a hole injection layer on the ITO glass substrate. As a hole transport layer, a film (200 Å) was formed of [HT]. Subsequently, a light-emitting layer (250 Å) was formed of a combination of the host compounds of the present disclosure and the dopant compound [BD-3], [BD-13], or [BD-26](2 wt %). Then, [Chemical Formula E-1] was deposited to form an electron transport layer (300 Å) on which Liq was deposited to form an electron injection layer (10 Å) and then covered with an Al layer as a cathode (1000 Å) to fabricate an organic light-emitting diode. The organic light-emitting diodes thus obtained were measured at 10 mA/cm² for luminescence properties and the measurements are summarized in Table 1, below.

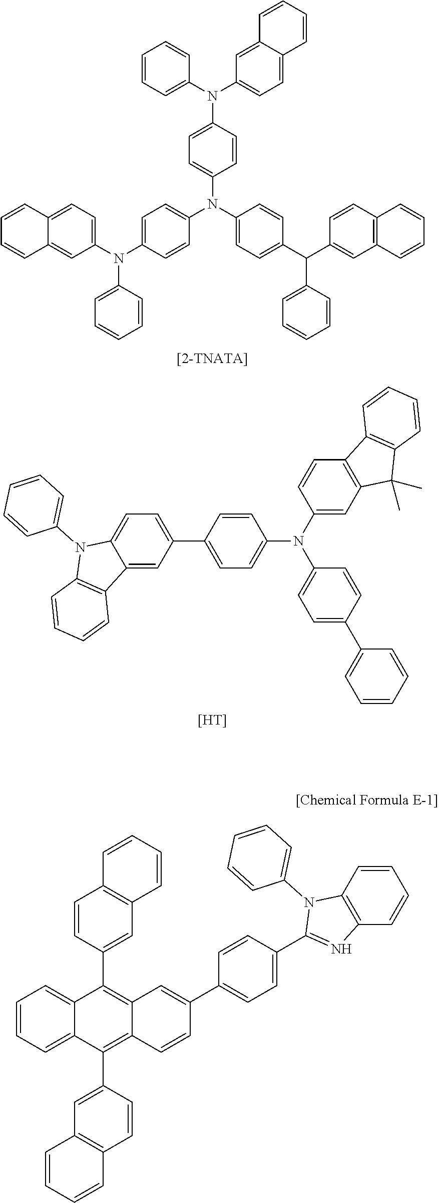

### Comparative Examples 1 to 8

Organic light-emitting diodes were fabricated in the same manner as in the Examples, with the exception of using one of the following [RH-1] and [RH-2], instead of the compounds of the Examples, as a host, or [RD-1] as a dopant for the light-emitting layers. The organic light-emitting diodes were measured for emission properties at 10 mA/cm². Structures of [RH-1], [RH-2], and [RD-1] are as follows.

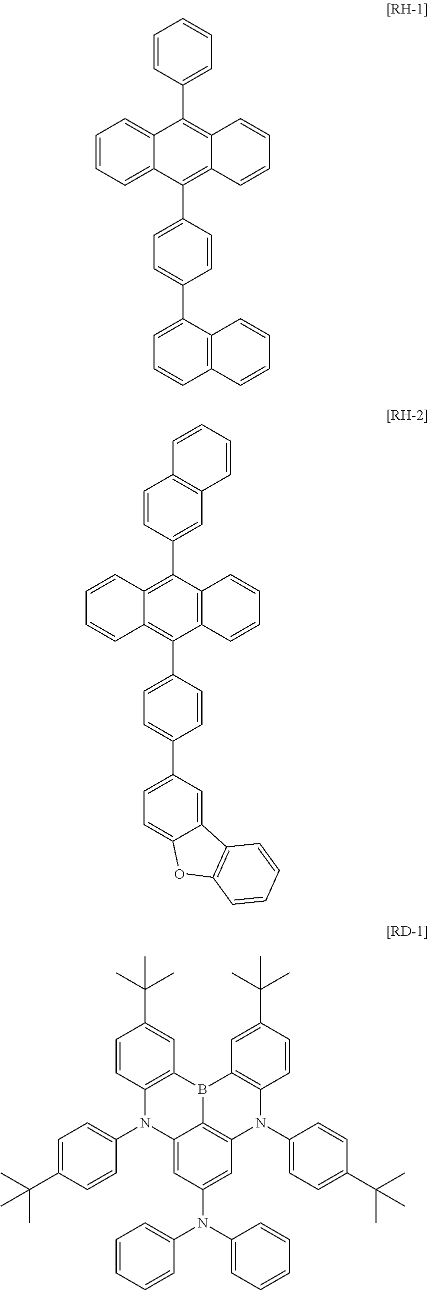

TABLE 1

| | | | External quantum | Life span |
|---|---|---|---|---|
| | Host | Dopant | efficiency (%) | (T97, hr) |
| Ex. 1 | BH-1 | BD-3 | 10.3 | 185 |
| Ex. 2 | BH-2 | BD-3 | 9.5 | 225 |
| Ex. 3 | BH-3 | BD-3 | 10.1 | 222 |
| Ex. 4 | BH-4 | BD-3 | 9.1 | 183 |
| Ex. 5 | BH-5 | BD-3 | 9.4 | 172 |
| Ex. 6 | BH-6 | BD-3 | 9.1 | 165 |
| Ex. 7 | BH-7 | BD-3 | 9.5 | 190 |
| Ex. 8 | BH-8 | BD-3 | 9.8 | 169 |
| Ex. 9 | BH-9 | BD-3 | 8.7 | 177 |
| Ex. 10 | BH-10 | BD-3 | 9.8 | 224 |
| Ex. 11 | BH-1 | BD-13 | 10.6 | 177 |
| Ex. 12 | BH-2 | BD-13 | 9.7 | 216 |
| Ex. 13 | BH-3 | BD-13 | 10.5 | 224 |
| Ex. 14 | BH-5 | BD-13 | 9.6 | 168 |
| Ex. 15 | BH-10 | BD-13 | 10.1 | 221 |
| Ex. 16 | BH-3 | BD-26 | 9.9 | 197 |
| Ex. 17 | BH-10 | BD-26 | 9.5 | 194 |
| C. Ex. 1 | RH-1 | BD-3 | 7.2 | 121 |
| C. Ex. 2 | RH-2 | BD-3 | 7.4 | 117 |
| C. Ex. 3 | RH-1 | BD-13 | 7.4 | 118 |
| C. Ex. 4 | RH-2 | BD-13 | 7.7 | 127 |
| C. Ex. 5 | RH-1 | RD-1 | 5.7 | 70 |
| C. Ex. 6 | RH-2 | RD-1 | 5.9 | 74 |
| C. Ex. 7 | BH-1 | RD-1 | 8.2 | 114 |
| C. Ex. 8 | BH-10 | RD-1 | 7.5 | 138 |

As is understood from the date of Table 1, the organic light-emitting diodes of the present disclosure were observed to exhibit higher efficiency and longevity, compared to conventional organic light-emitting diodes and thus is highly applicable as organic light-emitting diodes.

### INDUSTRIAL APPLICABILITY

When used as a host material in an organic light-emitting diode, the compound represented by Chemical Formula A according to the present disclosure provides high efficiency and longevity therefor, thus finding high applicability in the industries including organic light-emitting diodes and displays.

The invention claimed is:

1. An organic light-emitting diode, comprising:
a first electrode:
a second electrode; and
a light-emitting layer interposed between the first electrode and the second electrode,
wherein the light-emitting layer contains a host and a dopant, the host including at least one of the anthracene compounds represented by the following Chemical Formula A, the dopant including at least one of the polycyclic compounds represented by the following Chemical Formula 3 or 4: 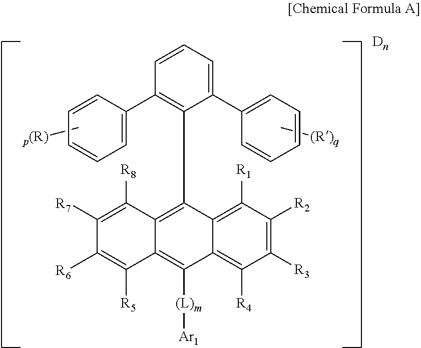
wherein,
R, R′, and R₁ to R₈, which are same or different, are each independently a hydrogen atom, a deuterium atom, a substituted or unsubstituted alkyl of 1 to 30 carbon atoms, a substituted or unsubstituted alkenyl of 2 to 30 carbon atoms, a substituted or unsubstituted aryl of 6 to 50 carbon atoms, a substituted or unsubstituted cycloalkyl of 3 to 30 carbon atoms, a substituted or unsubstituted heterocycloalkyl of 3 to 30 carbon atoms, a substituted or unsubstituted heteroaryl of 2 to 50 carbon atoms, a substituted or unsubstituted aromatic hydrocarbon ring-fused cycloalkyl of 7 to 30 carbon atoms, a substituted or unsubstituted heteroaromatic ring-fused cycloalkyl of 5 to 30 carbon atoms, a substituted or unsubstituted aromatic hydrocarbon ring-fused heterocycloalkyl of 6 to 30 carbon atoms, a substituted or unsubstituted aliphatic hydrocarbon ring-fused aryl of 7 to 30 carbon atoms, a substituted or unsubstituted aliphatic hydrocarbon ring-fused heteroaryl of 5 to 30 carbon atoms, a substituted or unsubstituted heteroaliphatic ring-fused aryl of 6 to 30 carbon atoms, a substituted or unsubstituted heteroaliphatic ring-fused heteroaryl of 5 to 30 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted amine of 0 to 30 carbon atoms, a substituted or unsubstituted silyl of 0 to 30 carbon atoms, a substituted or unsubstituted germanium of 0 to 30 carbon atoms, a nitro, a cyano, and a halogen,
L, which functions as a linker, is any one selected from a single bond, a substituted or unsubstituted arylene of 6 to 50 carbon atoms, a substituted or unsubstituted heteroarylene of 2 to 50 carbon atoms, a substituted or unsubstituted aromatic hydrocarbon ring-fused cycloalkylene of 7 to 30 carbon atoms, and a substituted or unsubstituted aliphatic hydrocarbon ring-fused arylene of 8 to 30 carbon atoms,
m is an integer of 1 to 3, wherein when m is 2 or higher, the corresponding L's are same or different,
Ar₁ is any one selected from a substituted or unsubstituted aryl of 6 to 50 carbon atoms, a substituted or unsubstituted heteroaryl of 2 to 50 carbon atoms, a substituted or unsubstituted cycloalkyl of 3 to 30 carbon atoms, a substituted or unsubstituted heterocycloalkyl of 3 to 30 carbon atoms, a substituted or unsubstituted aromatic hydrocarbon ring-fused cycloalkyl of 7 to 30 carbon atoms, a substituted or unsubstituted heteroaromatic ring-fused cycloalkyl of 5 to 30 carbon atoms, a substituted or unsubstituted aromatic hydrocarbon ring-fused heterocycloalkyl of 6 to 30 carbon atoms, a substituted or unsubstituted aliphatic hydrocarbon ring-fused aryl of 7 to 30 carbon atoms, a substituted or unsubstituted aliphatic hydrocarbon ring-fused heteroaryl of 5 to 30 carbon atoms, a substituted or unsubstituted heteroaliphatic ring-fused aryl of 6 to 30 carbon atoms, and a substituted or unsubstituted heteroaliphatic ring-fused heteroaryl of 5 to 30 carbon atoms,
Dₙ stands for a number (n) of deuterium atoms substituted for hydrogen atoms in Chemical Formula A,
n is an integer of 0 to 60,
p and q, which are same or different, are each independently an integer of 1 to 5, where when p or q is 2 or higher, the corresponding R or R′ radicals are same or different,
the plural R radicals can be connected to each other to additionally form an aliphatic or aromatic mono- or polycyclic ring, and
the plural R′ radicals can be connected to each other to additionally form an aliphatic or aromatic mono- or polycyclic ring; 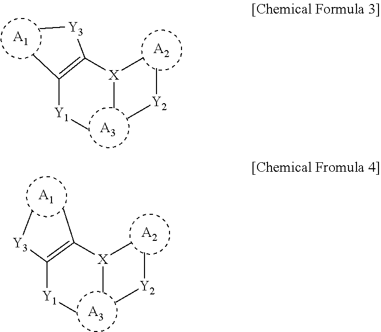
wherein,
X is B, P=O, P=S, or Al,
Y₁ and Y₂, which are same or different, are each independently any one selected from NR₁₁, O, S, CR₁₂R₁₃, SiR₁₄R₁₅, and GeR₁₆R₁₇,
Y₃ is O, S, or NR₁₀,
A₁ to A₃, which are same or different, are each independently a substituted or unsubstituted aromatic hydrocarbon ring of 6 to 50 carbon atoms, a substituted or unsubstituted aliphatic hydrocarbon ring-fused aromatic hydrocarbon ring of 6 to 50 carbon atoms, a substituted or unsubstituted heteroaromatic ring of 2 to 50 carbon atoms, and a substituted or unsubstituted aliphatic hydrocarbon ring-fused heteroaromatic ring of 2 to 50 carbon atoms,
R₁₀ to R₁₇, which are same or different, are each independently any one selected from a hydrogen atom, a deuterium atom, a substituted or unsubstituted alkyl of 1 to 30 carbon atoms, a substituted or unsubstituted alkenyl of 2 to 30 carbon atoms, a substituted or unsubstituted aryl of 6 to 50 carbon atoms, a substituted or unsubstituted cycloalkyl of 3 to 30 carbon atoms, a substituted or unsubstituted heterocycloalkyl of 3 to 30 carbon atoms, a substituted or unsubstituted heteroaryl of 2 to 50 carbon atoms, a substituted or unsubstituted aromatic hydrocarbon ring-fused cycloalkyl of 7 to 30 carbon atoms, a substituted or unsubstituted heteroaromatic ring-fused cycloalkyl of 5 to 30 carbon atoms, a substituted or unsubstituted aromatic hydrocarbon ring-fused heterocycloalkyl of 6 to 30 carbon atoms, a substituted or unsubstituted aliphatic hydrocarbon ring-fused aryl of 7 to 30 carbon atoms, a substituted or unsubstituted aliphatic hydrocarbon ring-fused heteroaryl of 5 to 30 carbon atoms, a substituted or unsubstituted heteroaliphatic ring-fused aryl of 6 to 30 carbon atoms, a substituted or unsubstituted heteroaliphatic ring-fused heteroaryl of 5 to 30 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted amine of 0 to 30 carbon atoms, a substituted or unsubstituted silyl of 0 to 30 carbon atoms, a substituted or unsubstituted germanium of 0 to 30 carbon atoms, a nitro, a cyano, and a halogen,
at least one of R₁₀ to R₁₇ can be connected to any one of the A₁ to A₃ ring moiety to additionally form an aliphatic or aromatic mono- or polycyclic ring,
a linkage can be formed between R₁₂ and R₁₃, between R₁₄ and R₁₅, and/or between R₁₆ and R₁₇ to additionally form an aliphatic or aromatic mono- or polycyclic ring,
wherein the term “substituted” in the expression “substituted or unsubstituted” used for the compounds of Chemical Formulas A, 3, and 4 means having at least one substituent selected from the group consisting of a deuterium atom, a cyano, a halogen, a hydroxy, a nitro, alkyl of 1 to 30 carbon atoms, a halogenated alkyl of 1 to 30 carbon atoms, an alkenyl of 2 to 30 carbon atoms, an alkynyl of 2 to 30 carbon atoms, a cycloalkyl of 3 to 30 carbon atoms, a heteroalkyl of 1 to 30 carbon atoms, an aryl of 6 to 30 carbon atoms, an arylalkyl of 7 to 30 carbon atoms, an alkylaryl of 7 to 30 carbon atoms, a heteroaryl of 2 to 30 carbon atoms, a heteroarylalkyl of 3 to 30 carbon atoms, an alkylheteroaryl of 3 to 30 carbon atoms, an alkoxy of 1 to 30 carbon atoms, an aromatic hydrocarbon ring-fused cycloalkyl of 7 to 30 carbon atoms, a heteroaromatic ring-fused cycloalkyl of 5 to 30 carbon atoms, an aromatic hydrocarbon ring-fused heterocycloalkyl of 6 to 30 carbon atoms, an aliphatic hydrocarbon ring-fused aryl of 7 to 30 carbon atoms, an aliphatic hydrocarbon ring-fused heteroaryl of 5 to 30 carbon atoms, a substituted or unsubstituted heteroaliphatic ring-fused aryl of 6 to 30 carbon atoms, a substituted or unsubstituted heteroaliphatic ring-fused heteroaryl of 5 to 30 carbon atoms, an amine of 1 to 40 carbon atoms, a silyl of 1 to 40 carbon atoms, a germanium of 1 to 40 carbon atoms, an aryloxy of 6 to 30 carbon atoms, and an arylthionyl of 6 to 30 carbon atoms, with at least one hydrogen atom on the substituent being substitutable with a deuterium atom.

2. An organic light-emitting diode, comprising:
a first electrode:
a second electrode; and
a light-emitting layer interposed between the first electrode and the second electrode,
wherein the light-emitting layer contains a host and a dopant, the host including at least one of the anthracene compounds represented by the following Chemical Formula A, the dopant including at least one of the polycyclic compounds represented by any one of the following Chemical Formula 3-1 to 4-3: 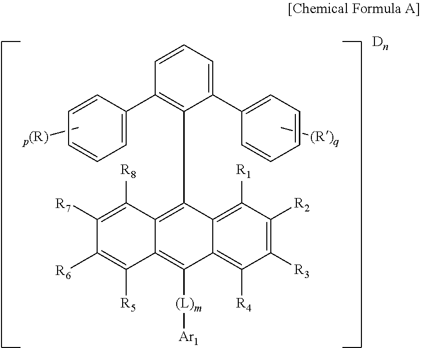
wherein
R, R′, and R₁ to R₈, which are same or different, are each independently any one selected from a hydrogen atom, a deuterium atom, a substituted or unsubstituted alkyl of 1 to 30 carbon atoms, a substituted or unsubstituted alkenyl of 2 to 30 carbon atoms, a substituted or unsubstituted aryl of 6 to 50 carbon atoms, a substituted or unsubstituted cycloalkyl of 3 to 30 carbon atoms, a substituted or unsubstituted heterocycloalkyl of 3 to 30 carbon atoms, a substituted or unsubstituted heteroaryl of 2 to 50 carbon atoms, a substituted or unsubstituted aromatic hydrocarbon ring-fused cycloalkyl of 7 to 30 carbon atoms, a substituted or unsubstituted heteroaromatic ring-fused cycloalkyl of 5 to 30 carbon atoms, a substituted or unsubstituted aromatic hydrocarbon ring-fused heterocycloalkyl of 6 to 30 carbon atoms, a substituted or unsubstituted aliphatic hydrocarbon ring-fused aryl of 7 to 30 carbon atoms, a substituted or unsubstituted aliphatic hydrocarbon ring-fused heteroaryl of 5 to 30 carbon atoms, a substituted or unsubstituted heteroaliphatic ring-fused aryl of 6 to 30 carbon atoms, a substituted or unsubstituted heteroaliphatic ring-fused heteroaryl of 5 to 30 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted amine of 0 to 30 carbon atoms, a substituted or unsubstituted silyl of 0 to 30 carbon atoms, a substituted or unsubstituted germanium of 0 to 30 carbon atoms, a nitro, a cyano, and a halogen,
L, which functions as a linker, is any one selected from a single bond, a substituted or unsubstituted arylene of 6 to 50 carbon atoms, a substituted or unsubstituted heteroarylene of 2 to 50 carbon atoms, a substituted or unsubstituted aromatic hydrocarbon ring-fused cycloalkylene of 7 to 30 carbon atoms, and a substituted or unsubstituted aliphatic hydrocarbon ring-fused arylene of 8 to 30 carbon atoms,
m is an integer of 1 to 3, wherein when m is 2 or higher, the corresponding L's are same or different,
Ar₁ is any one selected from a substituted or unsubstituted aryl of 6 to 50 carbon atoms, a substituted or unsubstituted heteroaryl of 2 to 50 carbon atoms, a substituted or unsubstituted cycloalkyl of 3 to 30 carbon atoms, a substituted or unsubstituted heterocycloalkyl of 3 to 30 carbon atoms, a substituted or unsubstituted aromatic hydrocarbon ring-fused cycloalkyl of 7 to 30 carbon atoms, a substituted or unsubstituted heteroaromatic ring-fused cycloalkyl of 5 to 30 carbon atoms, a substituted or unsubstituted aromatic hydrocarbon ring-fused heterocycloalkyl of 6 to 30 carbon atoms, a substituted or unsubstituted aliphatic hydrocarbon ring-fused aryl of 7 to 30 carbon atoms, a substituted or unsubstituted aliphatic hydrocarbon ring-fused heteroaryl of 5 to 30 carbon atoms, a substituted or unsubstituted heteroaliphatic ring-fused aryl of 6 to 30 carbon atoms, and a substituted or unsubstituted heteroaliphatic ring-fused heteroaryl of 5 to 30 carbon atoms,
Dₙ stands for a number (n) of deuterium atoms substituted for hydrogen atoms in Chemical Formula A,
n is an integer of 0 to 60,
p and q, which are same or different, are each independently an integer of 1 to 5, where when p or q is 2 or higher, the corresponding R or R′ radicals are same or different,
the plural R radicals may be connected to each other to additionally form an aliphatic or aromatic mono- or polycyclic ring, and
the plural R′ radicals may be connected to each other to additionally form an aliphatic or aromatic mono- or polycyclic ring; 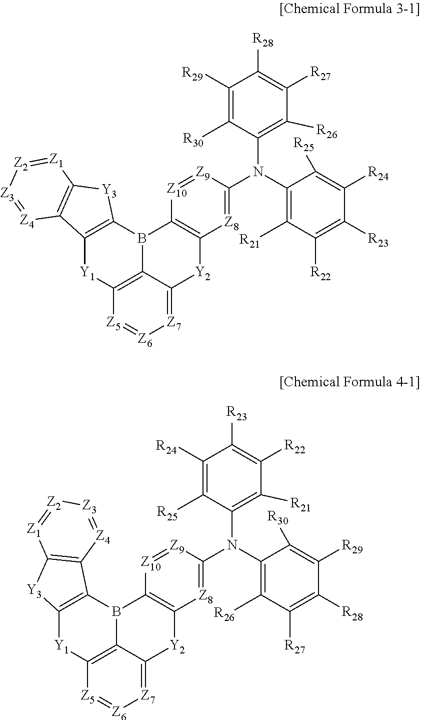
wherein
Z₁ to Z₁₀, which are same or different, are each independently CR₃₁ or N, when at least two of Z₁ to Z₁₀ are each CR₃₁, the corresponding CR₃₁'s are same or different,
when at least two of Z₁ to Z₁₀ are each CR₃₁, the corresponding R₃₁'s are connected to additionally form an aliphatic or aromatic mono- or polycyclic ring,
R₂₁ to R₃₁, which are same or different, are each independently any one selected from a hydrogen atom, a deuterium atom, an unsubstituted alkyl of 1 to 24 carbon atoms, an unsubstituted alkynyl of 2 to 24 carbon atoms, an unsubstituted alkenyl of 2 to 24 carbon atoms, an unsubstituted aryl of 6 to 24 carbon atoms, an unsubstituted alkylaryl of 7 to 24 carbon atoms, an unsubstituted cycloalkyl of 3 to 24 carbon atoms, an unsubstituted unsubstituted heterocycloalkyl of 3 to 24 carbon atoms, an uunsubstituted heteroaryl of 2 to 24 carbon atoms, an unsubstituted alkylheteroaryl of 3 to 24 carbon atoms, an unsubstituted aromatic hydrocarbon ring-fused cycloalkyl of 7 to 24 carbon atoms, an unsubstituted heteroaromatic ring-fused cycloalkyl of 5 to 24 carbon atoms, an unsubstituted aromatic hydrocarbon ring-fused heterocycloalkyl of 6 to 24 carbon atoms, an unsubstituted aliphatic hydrocarbon ring-fused aryl of 7 to 24 carbon atoms, an unsubstituted aliphatic hydrocarbon ring-fused heteroaryl of 5 to 24 carbon atoms, an unsubstituted heteroaliphatic ring-fused aryl of 6 to 24 carbon atoms, an unsubstituted heteroaliphatic ring-fused heteroaryl of 5 to 24 carbon atoms, an unsubstituted alkoxy of 1 to 24 carbon atoms, an unsubstituted aryloxy of 6 to 24 carbon atoms, an unsubstituted alkylthioxy of 1 to 24 carbon atoms, an unsubstituted arylthioxy of 5 to 24 carbon atoms, an unsubstituted amine of 1 to 24 carbon atoms, an unsubstituted silyl of 1 to 24 carbon atoms, an unsubstituted germanium of 1 to 24 carbon atoms, a nitro, a cyano, and a halogen,
R₂₁ to R₃₁ may have at least one deuterium atom instead of a hydrogen atom,
adjacent radicals among R₂₁ to R₃₀ may be connected to each other to additionally form an aliphatic or aromatic mono- or polycyclic ring,
when at least one of Z₁ to Z₁₀ is CR₃₁, at least one of R₂₁, R₂₅, R₂₆, and R₃₀ may be connected to the corresponding CR₃₁ to additionally form an aliphatic or aromatic mono- or polycyclic ring,
Y₁ to Y₃, which are same or different, are each independently as defined in Chemical Formulas 3 and 4 or a linker represented by the following Structural Formula A,
with a proviso that at least one of Y₁ and Y₂ is a linker represented by the following Structural Formula A: 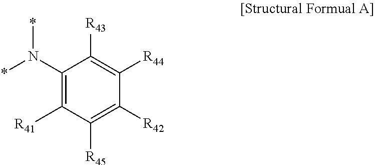
wherein,
R₄₁ to R₄₅, which are same or different, are each independently any one selected from a substituted or unsubstituted alkyl of 1 to 30 carbon atoms, a substituted or unsubstituted alkynyl of 2 to 30 carbon atoms, a substituted or unsubstituted alkenyl of 2 to 30 carbon atoms, a substituted or unsubstituted aryl of 6 to 50 carbon atoms, a substituted or unsubstituted cycloalkyl of 3 to 30 carbon atoms, a substituted or unsubstituted cycloalkenyl of 3 to 30 carbon atoms, a substituted or unsubstituted heterocycloalkyl of 1 to 30 carbon atoms, a substituted or unsubstituted heteroaryl of 2 to 50 carbon atoms, a substituted or unsubstituted aromatic hydrocarbon ring-fused cycloalkyl of 7 to 30 carbon atoms, a substituted or unsubstituted heteroaromatic ring-fused cycloalkyl of 5 to 30 carbon atoms, a substituted or unsubstituted aromatic hydrocarbon ring-fused heterocycloalkyl of 6 to 30 carbon atoms, a substituted or unsubstituted aliphatic hydrocarbon ring-fused aryl of 7 to 30 carbon atoms, a substituted or unsubstituted aliphatic hydrocarbon ring-fused heteroaryl of 5 to 30 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted amine of 0 to 30 carbon atoms, a substituted or unsubstituted silyl of 0 to 30 carbon atoms, a substituted or unsubstituted germanium of 0 to 30 carbon atoms, a nitro, a cyano, and a halogen,
at least one hydrogen atom in R₄₁ to R₄₅ is substitutable with a deuterium atom,
R₄₁ to R₄₅ may be connected to an adjacent substituent to additionally form an aliphatic or aromatic mono- or polycyclic ring; 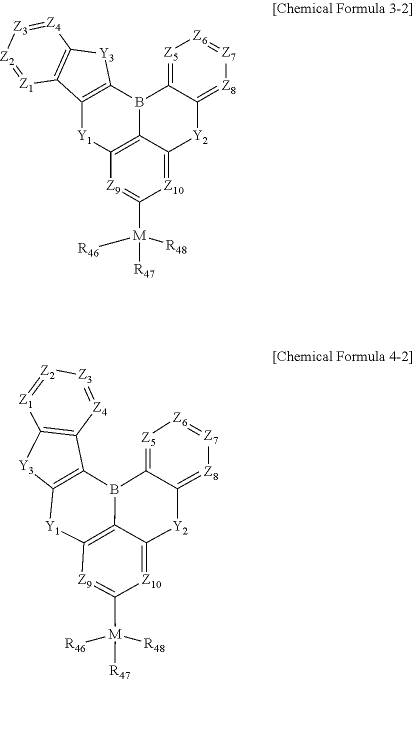
Y₁ to Y₃, which are same or different, are each independently as defined in Chemical Formulas 3 and 4,
Z₁ to Z₁₀, which are same or different, are each independently CR₃₁ or N,
when at least two of Z₁ to Z₁₀ are each CR₃₁, the corresponding CR₃₁'s are same or different,
when at least two of Z₁ to Z₈ are each CR₃₁, the corresponding R₃₁'s may be connected to each other to additionally form an aliphatic or aromatic mono- or polycyclic ring,
M is Si or Ge,
R₃₁ and R₄₆ to R₄₈, which are same or different, are each independently any one selected from a hydrogen atom, a deuterium atom, a substituted or unsubstituted alkyl of 1 to 30 carbon atoms, a substituted or unsubstituted alkynyl of 2 to 30 carbon atoms, a substituted or unsubstituted alkenyl of 2 to 30 carbon atoms, a substituted or unsubstituted aryl of 6 to 50 carbon atoms, a substituted or unsubstituted cycloalkyl of 3 to 30 carbon atoms, a substituted or unsubstituted cycloalkenyl of 3 to 30 carbon atoms, a substituted or unsubstituted heterocycloalkyl of 3 to 30 carbon atoms, a substituted or unsubstituted heteroaryl of 2 to 50 carbon atoms, a substituted or unsubstituted aromatic hydrocarbon ring-fused cycloalkyl of 7 to 30 carbon atoms, a substituted or unsubstituted heteroaromatic ring-fused cycloalkyl of 5 to 30 carbon atoms, a substituted or unsubstituted aromatic hydrocarbon ring-fused heterocycloalkyl of 6 to 30 carbon atoms, a substituted or unsubstituted aliphatic hydrocarbon ring-fused aryl of 7 to 30 carbon atoms, a substituted or unsubstituted aliphatic hydrocarbon ring-fused heteroaryl of 5 to 30 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted amine of 0 to 30 carbon atoms, a substituted or unsubstituted silyl of 0 to 30 carbon atoms, a substituted or unsubstituted germanium of 0 to 30 carbon atoms, a nitro, a cyano, and a halogen
at least one hydrogen atom in R₄₆ to R₄₀ is substitutable with a deuterium atom, and
R₄₆ to R₄₀ may each be connected to additionally form an aliphatic or aromatic mono- or polycyclic ring; 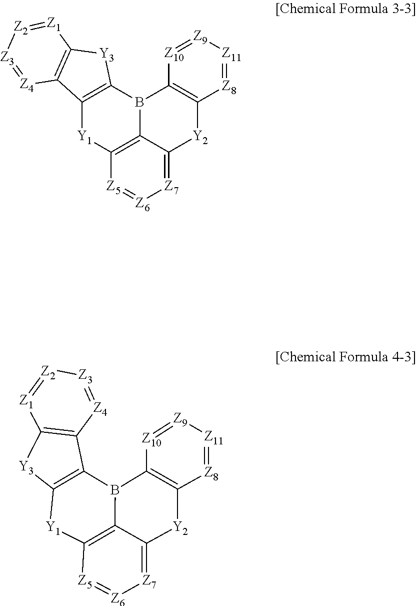
wherein,
Z₁ to Z₁₁, which are same or different, are each independently CR₃₁ or N,
when at least two of Z₁ to Z₁₁ are each CR₃₁, the corresponding CR₃₁'s are same or different,
when at least two of Z₁ to Z₁₁ are each CR₃₁, the corresponding R₃₁'s may be connected to each other to additionally form an aliphatic or aromatic mono- or polycyclic ring,
Y₁ to Y₃, which are same or different, are each independently as defined in Chemical Formulas 3 and 4, or a linker represented by the following Structural Formula B,
with a proviso that at least one of Y₁ and Y₂ is a linker represented by the 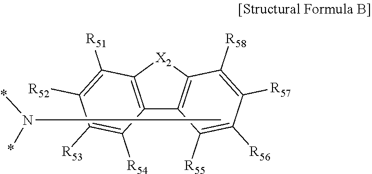
in Structural Formula B and Chemical Formulas 3-3 and 4-3,
X₂ is O or S,
R₃₁ and R₅₁ to R₅₈, which are same or different, are each independently any one selected from a hydrogen atom, a deuterium atom, a substituted or unsubstituted alkyl of 1 to 30 carbon atoms, a substituted or unsubstituted alkynyl of 2 to 30 carbon atoms, a substituted or unsubstituted alkenyl of 2 to 30 carbon atoms, a substituted or unsubstituted aryl of 6 to 50 carbon atoms, a substituted or unsubstituted cycloalkyl of 3 to 30 carbon atoms, a substituted or unsubstituted cycloalkenyl of 3 to 30 carbon atoms, a substituted or unsubstituted heterocycloalkyl of 1 to 30 carbon atoms, a substituted or unsubstituted heteroaryl of 2 to 50 carbon atoms, a substituted or unsubstituted aromatic hydrocarbon ring-fused cycloalkyl of 7 to 30 carbon atoms, a substituted or unsubstituted heteroaromatic ring-fused cycloalkyl of 5 to 30 carbon atoms, a substituted or unsubstituted aromatic hydrocarbon ring-fused heterocycloalkyl of 6 to 30 carbon atoms, a substituted or unsubstituted aliphatic hydrocarbon ring-fused aryl of 7 to 30 carbon atoms, a substituted or unsubstituted aliphatic hydrocarbon ring-fused heteroaryl of 5 to 30 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted amine of 0 to 30 carbon atoms, a substituted or unsubstituted silyl of 0 to 30 carbon atoms, a substituted or unsubstituted germanium of 0 to 30 carbon atoms, a nitro, a cyano, and a halogen,
at least one hydrogen atom in R₅₁ to R₅₈ is substitutable with a deuterium atom,
any one of R₅₁ to R₅₈ is a single bond linking to the nitrogen atom,
adjacent radicals among R₅₁ to R₅₈, except for the single bond, may be connected to each other to additionally form an aliphatic or aromatic mono- or polycyclic ring,
wherein the term “substituted” in the expression “substituted or unsubstituted” used for compounds of Chemical Formulas A and 3-1 to 4-3 means having at least one substituent selected from the group consisting of a deuterium, a cyano, a halogen, a hydroxy, a nitro, alkyl of 1 to 30 carbon atoms, a halogenated alkyl of 1 to 30 carbon atoms, an alkenyl of 2 to 30 carbon atoms, an alkynyl of 2 to 30 carbon atoms, a cycloalkyl of 3 to 30 carbon atoms, a heteroalkyl of 1 to 30 carbon atoms, an aryl of 6 to 30 carbon atoms, an arylalkyl of 7 to 30 carbon atoms, an alkylaryl of 7 to 30 carbon atoms, a heteroaryl of 2 to 30 carbon atoms, a heteroarylalkyl of 3 to 30 carbon atoms, an alkylheteroaryl of 3 to 30 carbon atoms, an alkoxy of 1 to 30 carbon atoms, an aromatic hydrocarbon ring-fused cycloalkyl of 7 to 30 carbon atoms, heteroaromatic ring-fused cycloalkyl of 5 to 30 carbon atoms, aromatic hydrocarbon ring-fused heterocycloalkyl of 6 to 30 carbon atoms, an aliphatic hydrocarbon ring-fused aryl of 7 to 30 carbon atoms, an aliphatic hydrocarbon ring-fused heteroaryl of 5 to 30 carbon atoms, a substituted or unsubstituted heteroaliphatic ring-fused aryl of 6 to 30 carbon atoms, a substituted or unsubstituted heteroaliphatic ring-fused heteroaryl of 5 to 30 carbon atoms, an amine of 1 to 40 carbon atoms, a silyl of 1 to 40 carbon atoms, a germanium of 1 to 40 carbon atoms aryloxy of 6 to 30 carbon atoms, and an arylthionyl of 6 to 30 carbon atoms, with at least one hydrogen atom in each substituent being substitutable with a deuterium atom.

3. The organic light-emitting diode of claim 1 or 2, wherein the anthracene compound is an anthracene compound represented by any one of the following Chemical Formulas A-1 to A-4: 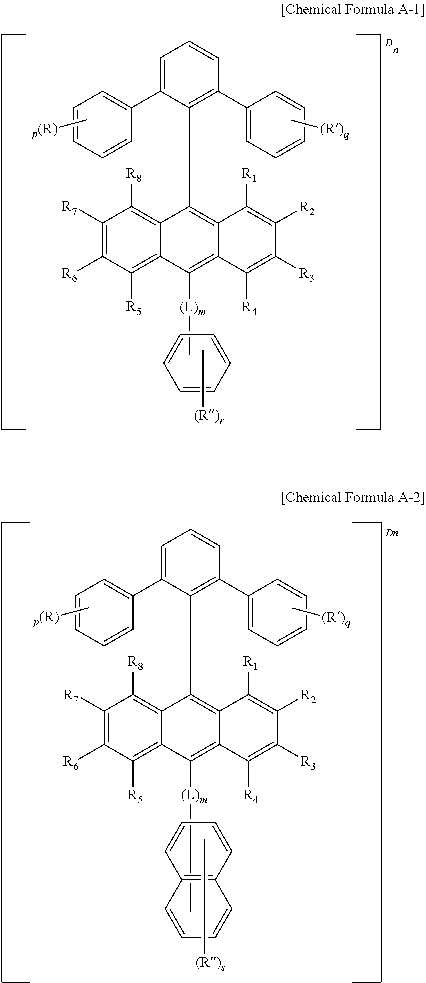 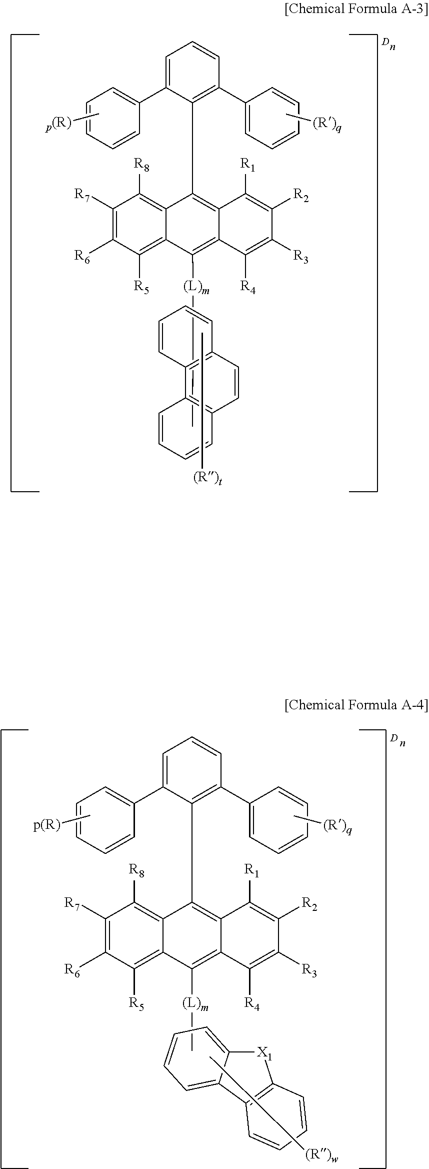
wherein
R, R′, R₁ to Re, L, m, Dₙ, p, and q are as defined in Chemical Formula A, R″ is any one selected from a hydrogen atom, a deuterium atom, a substituted or unsubstituted alkyl of 1 to 30 carbon atoms, a substituted or unsubstituted alkenyl of 2 to 30 carbon atoms, a substituted or unsubstituted aryl of 6 to 50 carbon atoms, a substituted or unsubstituted cycloalkyl of 3 to 30 carbon atoms, a substituted or unsubstituted heterocycloalkyl of 3 to 30 carbon atoms, a substituted or unsubstituted heteroaryl of 2 to 50 carbon atoms, a substituted or unsubstituted aromatic hydrocarbon ring-fused cycloalkyl of 7 to 30 carbon atoms, a substituted or unsubstituted heteroaromatic ring-fused cycloalkyl of 5 to 30 carbon atoms, a substituted or unsubstituted aromatic hydrocarbon ring-fused heterocycloalkyl of 6 to 30 carbon atoms, a substituted or unsubstituted aliphatic hydrocarbon ring-fused aryl of 7 to 30 carbon atoms, a substituted or unsubstituted aliphatic hydrocarbon ring-fused heteroaryl of 5 to 30 carbon atoms, a substituted or unsubstituted heteroaliphatic ring-fused aryl of 6 to 30 carbon atoms, a substituted or unsubstituted heteroaliphatic ring-fused heteroaryl of 5 to 30 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted amine of 0 to 30 carbon atoms, a substituted or unsubstituted silyl of 0 to 30 carbon atoms, a substituted or unsubstituted germanium of 0 to 30 carbon atoms, a nitro, a cyano, and a halogen,
r is an integer of 1 to 5, wherein when r is 2 or higher, the corresponding R″ radicals are same or different,
s is an integer of 1 to 7, wherein when s is 2 or higher, the corresponding R″ radicals are same or different,
t is an integer of 1 to 9, wherein when t is 2 or higher, the corresponding R″ radicals are same or different,
w is an integer of 1 to 7, wherein when w is 2 or higher, the corresponding R″ radicals are same or different,
in Chemical Formula A-4,
X₁ is a linker selected from O, S, CR₁₈R₁₉, and NR₂₀,
R₁₈ to R₂₀, which are same or different, are each independently any one selected from a substituted or unsubstituted alkyl of 1 to 30 carbon atoms and a substituted or unsubstituted aryl of 6 to 50 carbon atoms, and
R₁₈ and R₁₉ can be connected to each other to additionally form an aliphatic or aromatic mono- or polycyclic ring.

4. The organic light-emitting diode of claim 1 or 2, wherein R and R′ in Chemical Formula A is same or different and are each independently a hydrogen atom or a deuterium atom.

5. The organic light-emitting diode of claim 1 or 2, wherein L in Chemical Formula A is a single bond or any one selected from the following Structural Formulas 1 to 5: 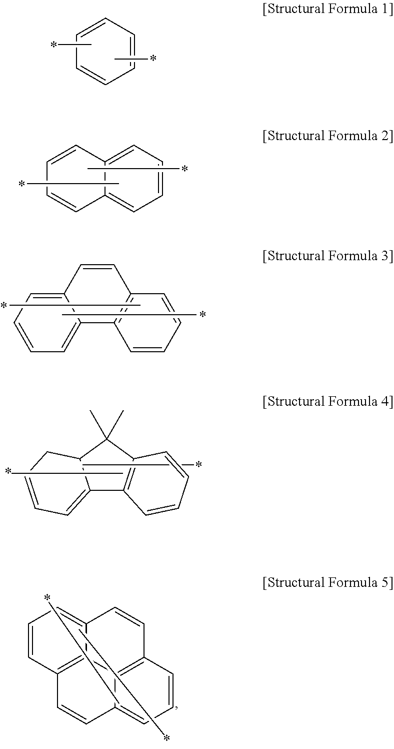
wherein a hydrogen atom or a deuterium atom can be bonded to an aromatic carbon atom of Structural Formulas 1 to 5.

6. The organic light-emitting diode of claim 1 or 2, wherein at least four of R₁ to R₈ in Chemical Formula A are each a deuterium atom and n is an integer of 4 to 50.

7. The organic light-emitting diode of claim 1 or 2, wherein Ar₁ in Chemical Formula A is a substituted or unsubstituted aryl of 6 to 20 carbon atoms or a substituted or unsubstituted heteroaryl of 3 to 20 carbon atoms.

8. The organic light-emitting diode of claim 3, wherein X₁ in Chemical Formula A-4 is O or S.

9. The organic light-emitting diode of claim 1 or 2, wherein the anthracene compound represented by Chemical Formula A is any one selected from the following compounds [BH-1] to [BH-100]:

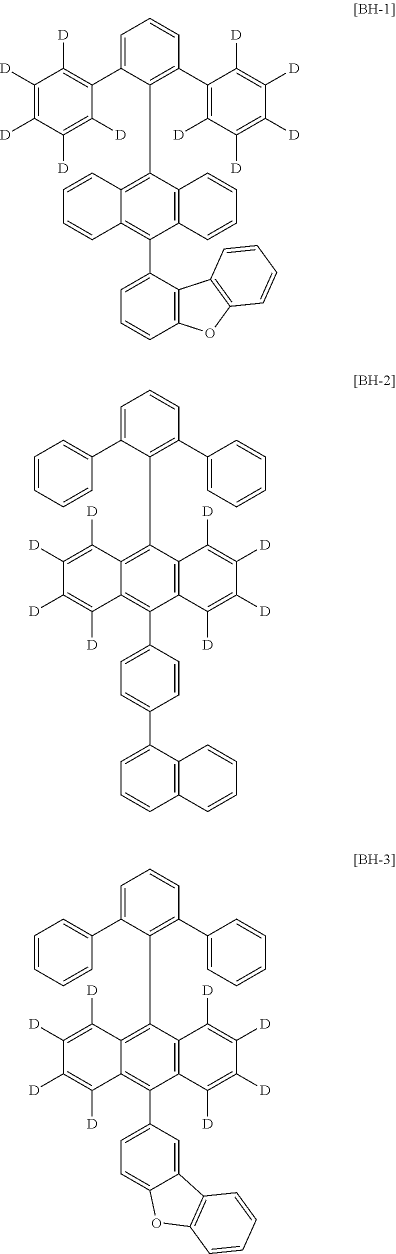 [SMILES: [2H]c1c([2H])c([2H])c(-c2cccc(-c3c([2H])c([2H])c([2H])c([2H])c3[2H])c2-c2c3ccccc3c(-c3cccc4oc5ccccc5c34)c3ccccc23)c([2H])c1[2H] ; [2H]c1c([2H])c([2H])c2c(-c3c(-c4ccccc4)cccc3-c3ccccc3)c3c([2H])c([2H])c([2H])c([2H])c3c(-c3ccc(-c4cccc5ccccc45)cc3)c2c1[2H] ; [2H]c1c([2H])c([2H])c2c(-c3c(-c4ccccc4)cccc3-c3ccccc3)c3c([2H])c([2H])c([2H])c([2H])c3c(-c3ccc4oc5ccccc5c4c3)c2c1[2H]]

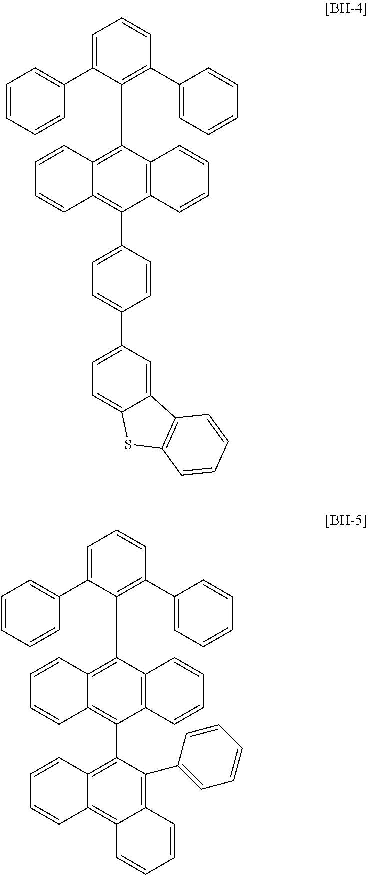

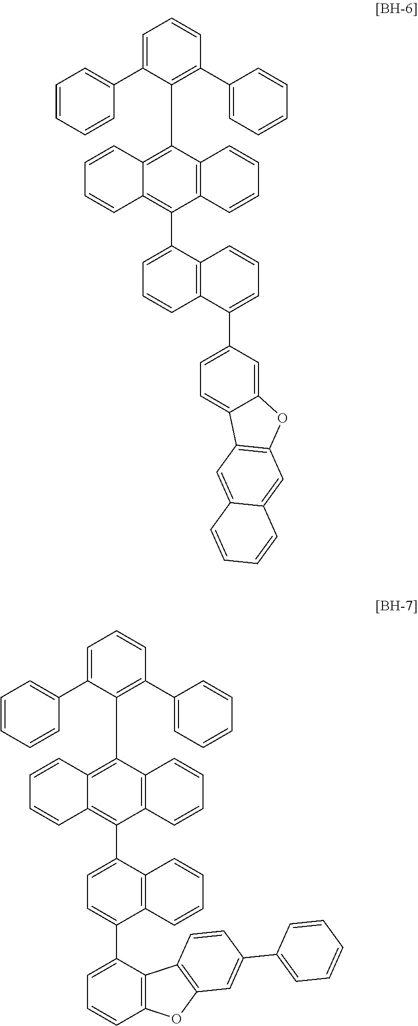 [SMILES: c1ccc(-c2cccc(-c3ccccc3)c2-c2c3ccccc3c(-c3cccc4c(-c5ccc6c(c5)oc5cc7ccccc7cc56)cccc34)c3ccccc23)cc1 ; c1ccc(-c2ccc3c(c2)oc2cccc(-c4ccc(-c5c6ccccc6c(-c6c(-c7ccccc7)cccc6-c6ccccc6)c6ccccc56)c5ccccc45)c23)cc1]

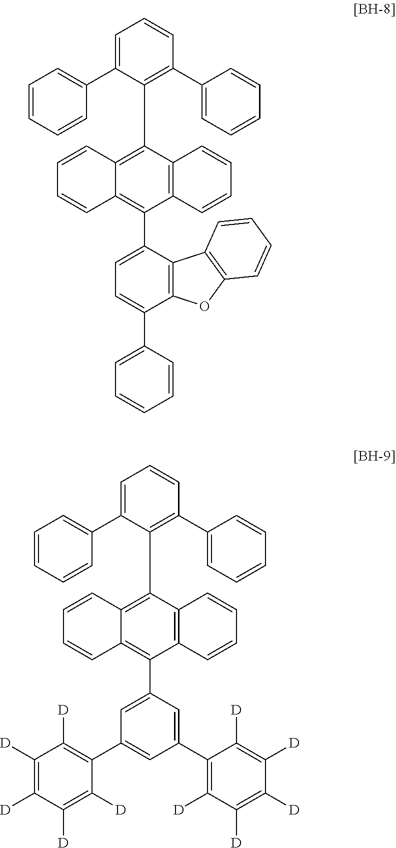 [SMILES: c1ccc(-c2cccc(-c3ccccc3)c2-c2c3ccccc3c(-c3ccc(-c4ccccc4)c4oc5ccccc5c34)c3ccccc23)cc1 ; [2H]c1c([2H])c([2H])c(-c2cc(-c3c([2H])c([2H])c([2H])c([2H])c3[2H])cc(-c3c4ccccc4c(-c4c(-c5ccccc5)cccc4-c4ccccc4)c4ccccc34)c2)c([2H])c1[2H]]

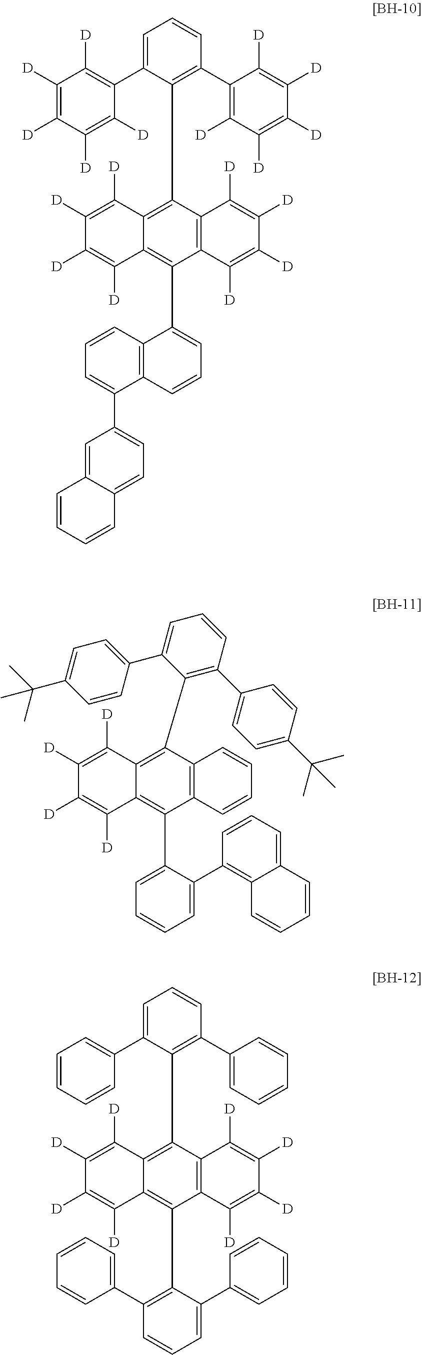 [SMILES: [2H]c1c([2H])c([2H])c(-c2cccc(-c3c([2H])c([2H])c([2H])c([2H])c3[2H])c2-c2c3c([2H])c([2H])c([2H])c([2H])c3c(-c3cccc4c(-c5ccc6ccccc6c5)cccc34)c3c([2H])c([2H])c([2H])c([2H])c23)c([2H])c1[2H] ; [2H]c1c([2H])c([2H])c2c(-c3c(-c4ccc(C(C)(C)C)cc4)cccc3-c3ccc(C(C)(C)C)cc3)c3ccccc3c(-c3ccccc3-c3cccc4ccccc34)c2c1[2H] ; [2H]c1c([2H])c([2H])c2c(-c3c(-c4ccccc4)cccc3-c3ccccc3)c3c([2H])c([2H])c([2H])c([2H])c3c(-c3c(-c4ccccc4)cccc3-c3ccccc3)c2c1[2H]]

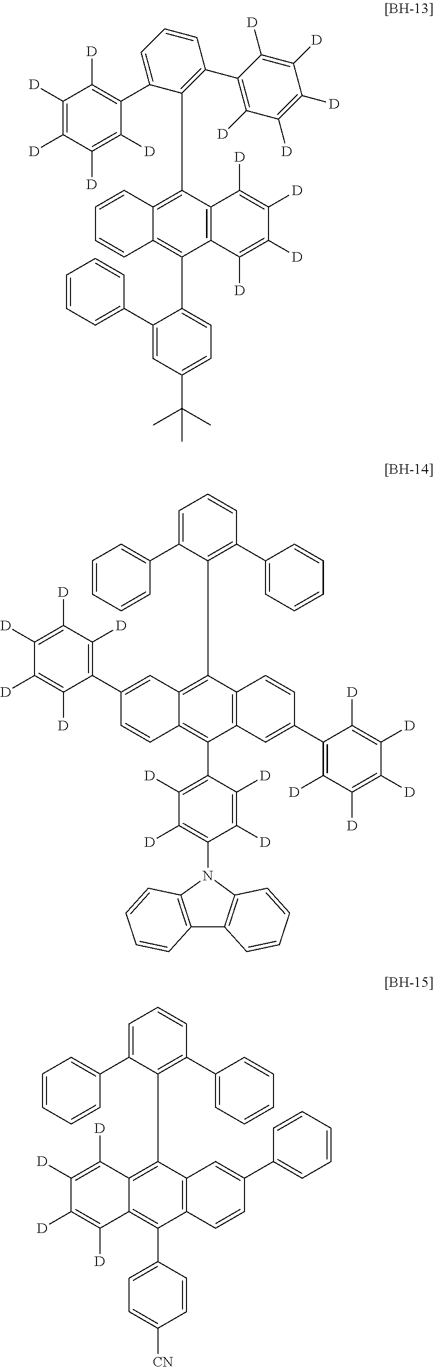 [SMILES: [2H]c1c([2H])c([2H])c(-c2cccc(-c3c([2H])c([2H])c([2H])c([2H])c3[2H])c2-c2c3ccccc3c(-c3ccc(C(C)(C)C)cc3-c3ccccc3)c3c([2H])c([2H])c([2H])c([2H])c23)c([2H])c1[2H] ; [2H]c1c([2H])c([2H])c(-c2ccc3c(-c4c([2H])c([2H])c(-n5c6ccccc6c6ccccc65)c([2H])c4[2H])c4cc(-c5c([2H])c([2H])c([2H])c([2H])c5[2H])ccc4c(-c4c(-c5ccccc5)cccc4-c4ccccc4)c3c2)c([2H])c1[2H] ; [2H]c1c([2H])c([2H])c2c(-c3c(-c4ccccc4)cccc3-c3ccccc3)c3cc(-c4ccccc4)ccc3c(-c3ccc(C#N)cc3)c2c1[2H]]

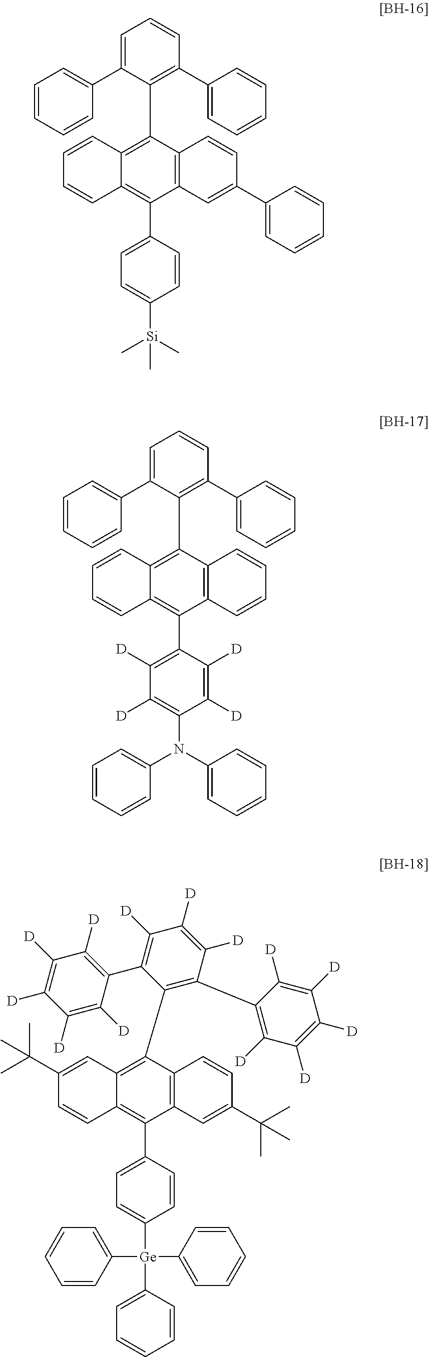

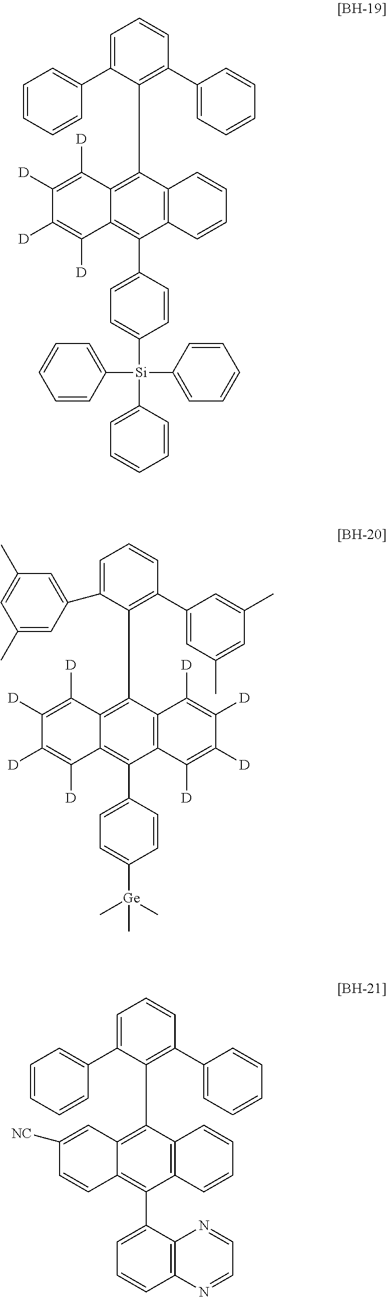

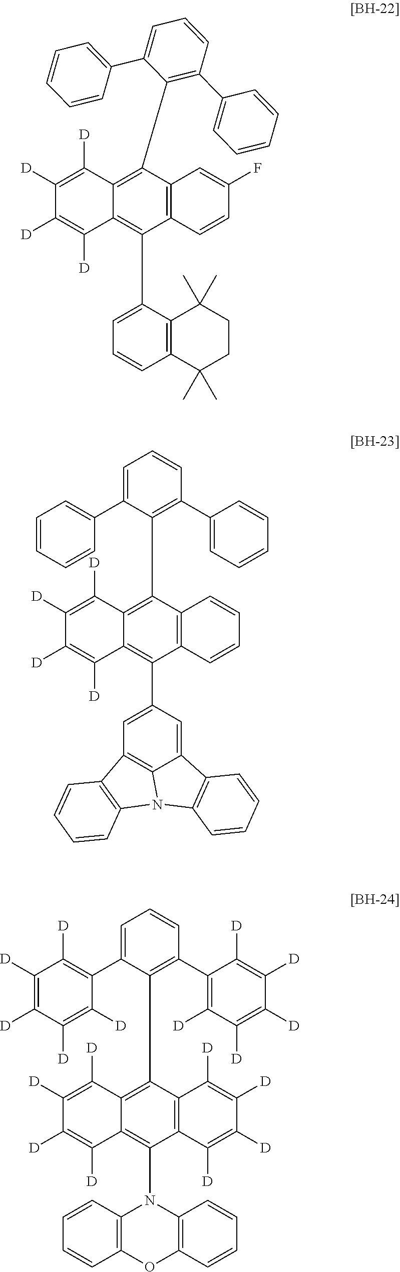

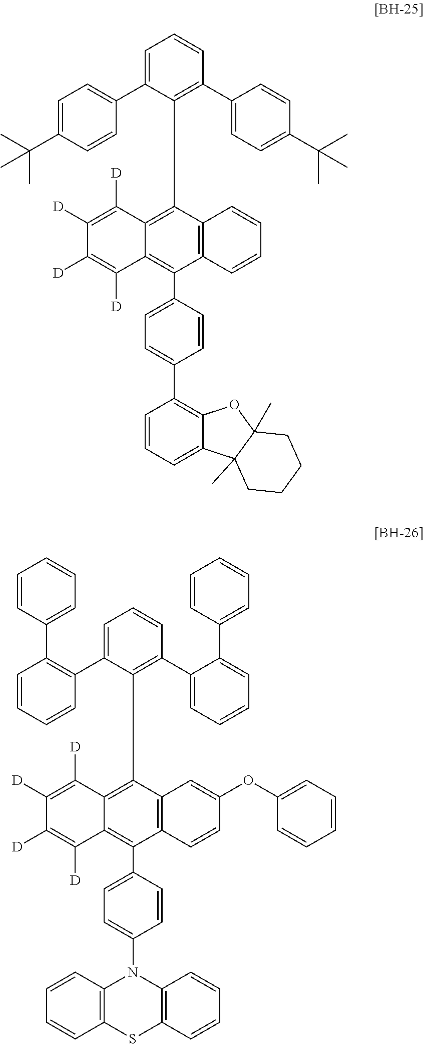

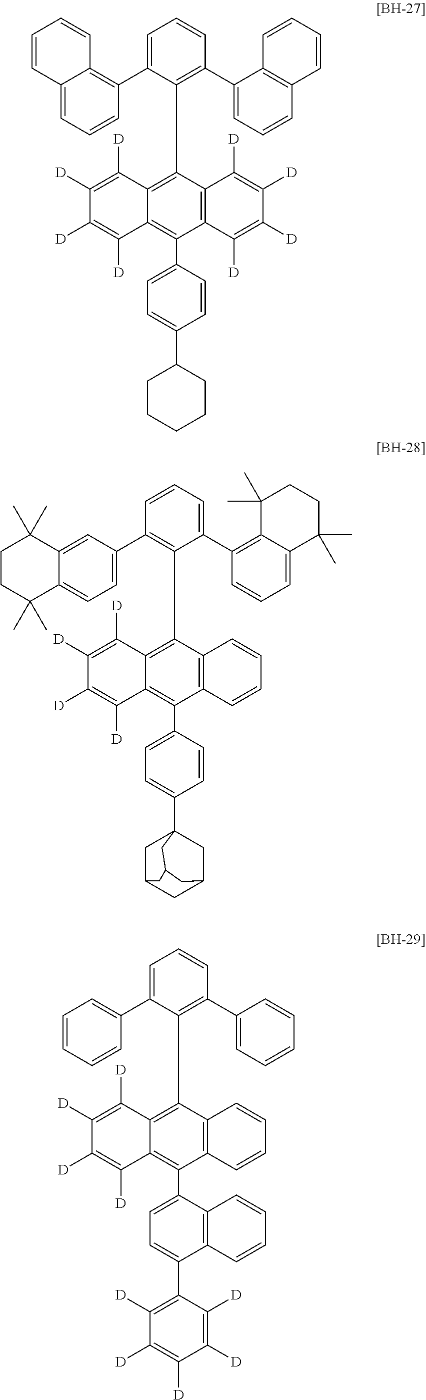 [SMILES: [2H]c1c([2H])c([2H])c2c(-c3c(-c4cccc5ccccc45)cccc3-c3cccc4ccccc34)c3c([2H])c([2H])c([2H])c([2H])c3c(-c3ccc(C4CCCCC4)cc3)c2c1[2H] ; [2H]c1c([2H])c([2H])c2c(-c3c(-c4ccc5c(c4)C(C)(C)CCC5(C)C)cccc3-c3cccc4c3C(C)(C)CCC4(C)C)c3ccccc3c(-c3ccc(C45CC6CC(CC(C6)C4)C5)cc3)c2c1[2H] ; [2H]c1c([2H])c([2H])c(-c2ccc(-c3c4ccccc4c(-c4c(-c5ccccc5)cccc4-c4ccccc4)c4c([2H])c([2H])c([2H])c([2H])c34)c3ccccc23)c([2H])c1[2H]]

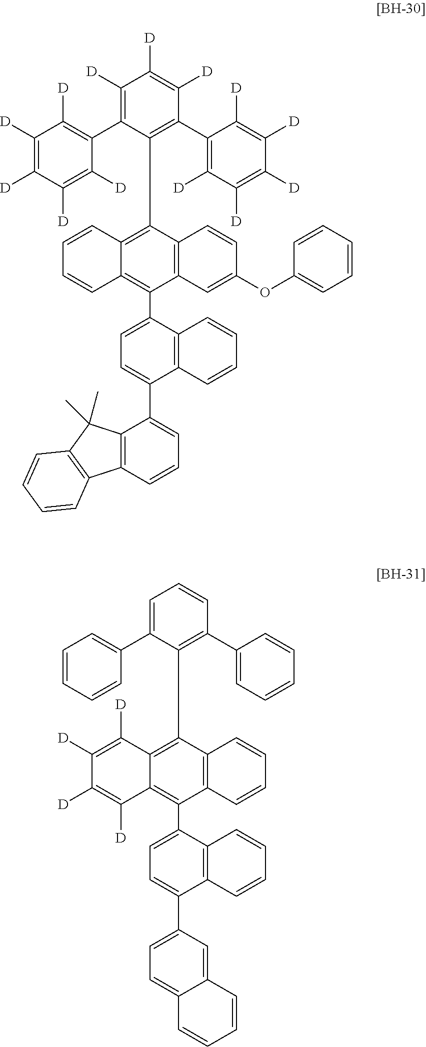 [SMILES: [2H]c1c([2H])c([2H])c(-c2c([2H])c([2H])c([2H])c(-c3c([2H])c([2H])c([2H])c([2H])c3[2H])c2-c2c3ccccc3c(-c3ccc(-c4cccc5c4C(C)(C)c4ccccc4-5)c4ccccc34)c3cc(Oc4ccccc4)ccc23)c([2H])c1[2H] ; [2H]c1c([2H])c([2H])c2c(-c3ccc(-c4ccc5ccccc5c4)c4ccccc34)c3ccccc3c(-c3c(-c4ccccc4)cccc3-c3ccccc3)c2c1[2H]]

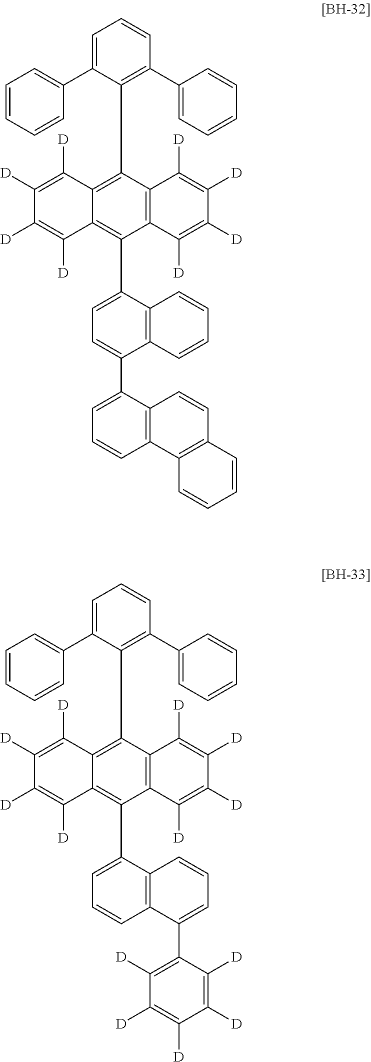 [SMILES: [2H]c1c([2H])c([2H])c2c(-c3ccc(-c4cccc5c4ccc4ccccc45)c4ccccc34)c3c([2H])c([2H])c([2H])c([2H])c3c(-c3c(-c4ccccc4)cccc3-c3ccccc3)c2c1[2H] ; [2H]c1c([2H])c([2H])c(-c2cccc3c(-c4c5c([2H])c([2H])c([2H])c([2H])c5c(-c5c(-c6ccccc6)cccc5-c5ccccc5)c5c([2H])c([2H])c([2H])c([2H])c45)cccc23)c([2H])c1[2H]]

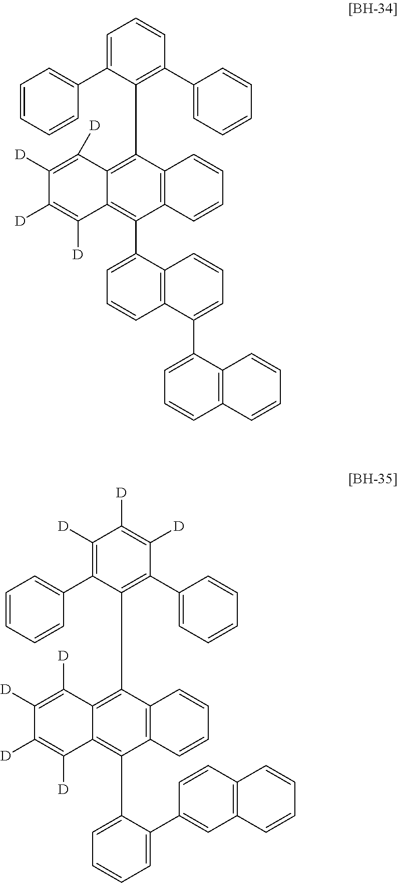 [SMILES: [2H]c1c([2H])c([2H])c2c(-c3cccc4c(-c5cccc6ccccc56)cccc34)c3ccccc3c(-c3c(-c4ccccc4)cccc3-c3ccccc3)c2c1[2H] ; [2H]c1c([2H])c(-c2ccccc2)c(-c2c3ccccc3c(-c3ccccc3-c3ccc4ccccc4c3)c3c([2H])c([2H])c([2H])c([2H])c23)c(-c2ccccc2)c1[2H]]

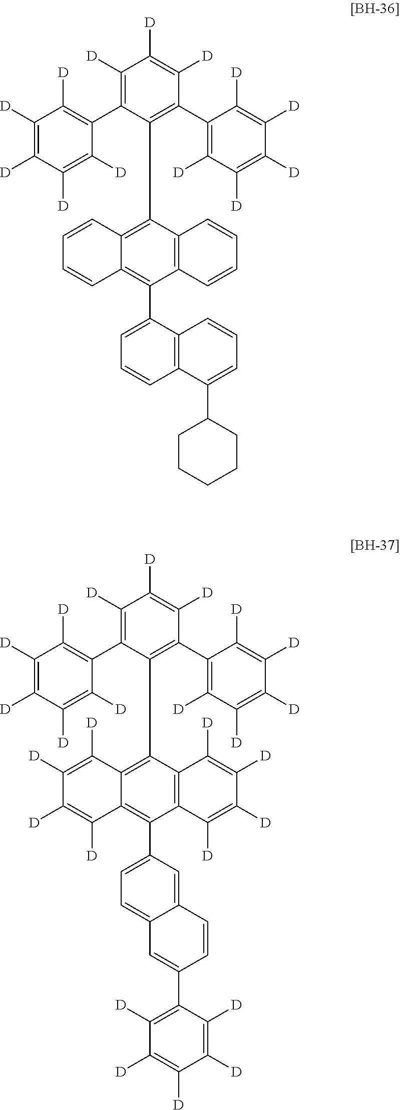 [SMILES: [2H]c1c([2H])c([2H])c(-c2c([2H])c([2H])c([2H])c(-c3c([2H])c([2H])c([2H])c([2H])c3[2H])c2-c2c3ccccc3c(-c3cccc4c(C5CCCCC5)cccc34)c3ccccc23)c([2H])c1[2H] ; [2H]c1c([2H])c([2H])c(-c2ccc3cc(-c4c5c([2H])c([2H])c([2H])c([2H])c5c(-c5c(-c6c([2H])c([2H])c([2H])c([2H])c6[2H])c([2H])c([2H])c([2H])c5-c5c([2H])c([2H])c([2H])c([2H])c5[2H])c5c([2H])c([2H])c([2H])c([2H])c45)ccc3c2)c([2H])c1[2H]]

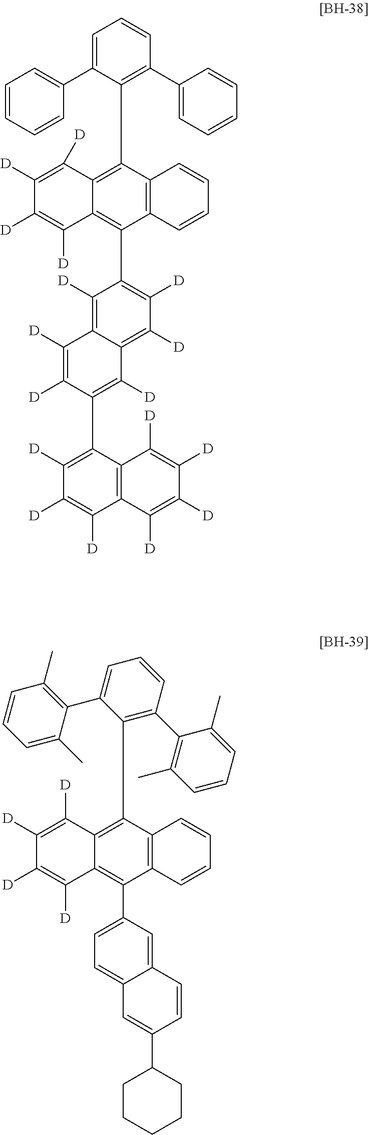 [SMILES: [2H]c1c(-c2c3ccccc3c(-c3c(-c4ccccc4)cccc3-c3ccccc3)c3c([2H])c([2H])c([2H])c([2H])c23)c([2H])c2c([2H])c([2H])c(-c3c([2H])c([2H])c([2H])c4c([2H])c([2H])c([2H])c([2H])c34)c([2H])c2c1[2H] ; [2H]c1c([2H])c([2H])c2c(-c3c(-c4c(C)cccc4C)cccc3-c3c(C)cccc3C)c3ccccc3c(-c3ccc4cc(C5CCCCC5)ccc4c3)c2c1[2H]]

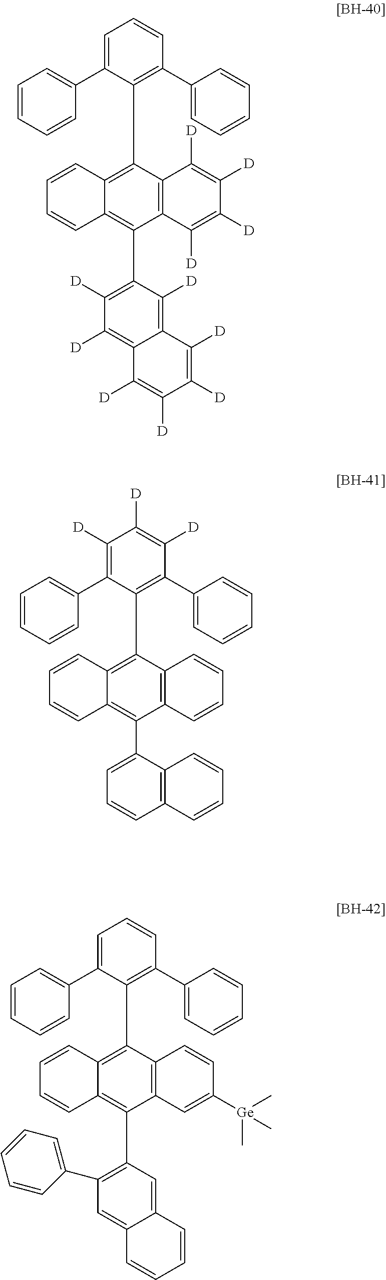

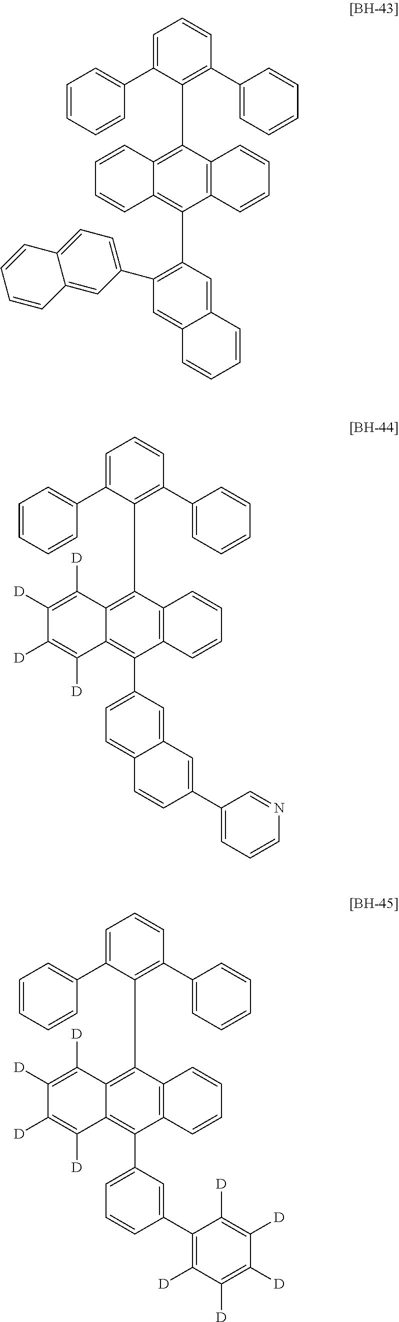

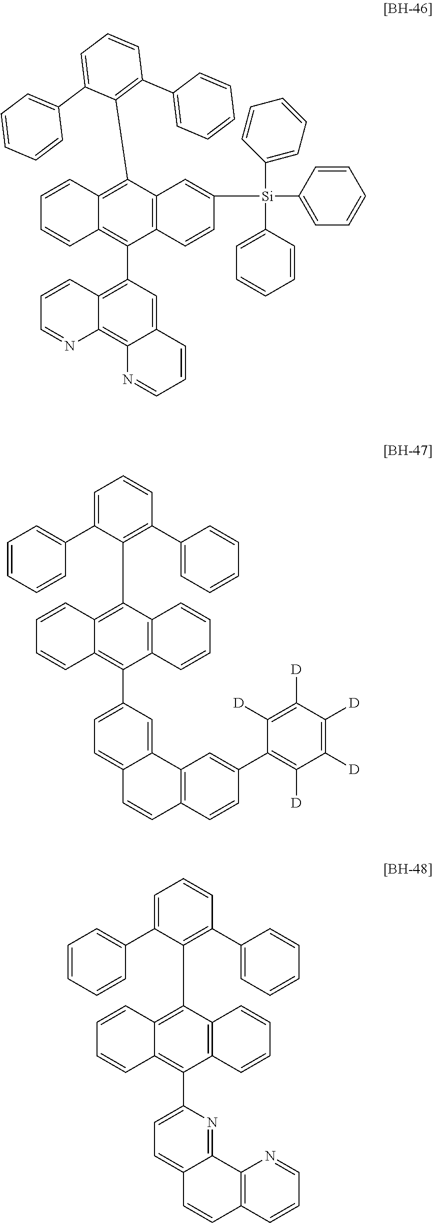 [SMILES: c1ccc(-c2cccc(-c3ccccc3)c2-c2c3ccccc3c(-c3cc4cccnc4c4ncccc34)c3ccc([Si](c4ccccc4)(c4ccccc4)c4ccccc4)cc23)cc1 ; [2H]c1c([2H])c([2H])c(-c2ccc3ccc4ccc(-c5c6ccccc6c(-c6c(-c7ccccc7)cccc6-c6ccccc6)c6ccccc56)cc4c3c2)c([2H])c1[2H] ; c1ccc(-c2cccc(-c3ccccc3)c2-c2c3ccccc3c(-c3ccc4ccc5cccnc5c4n3)c3ccccc23)cc1]

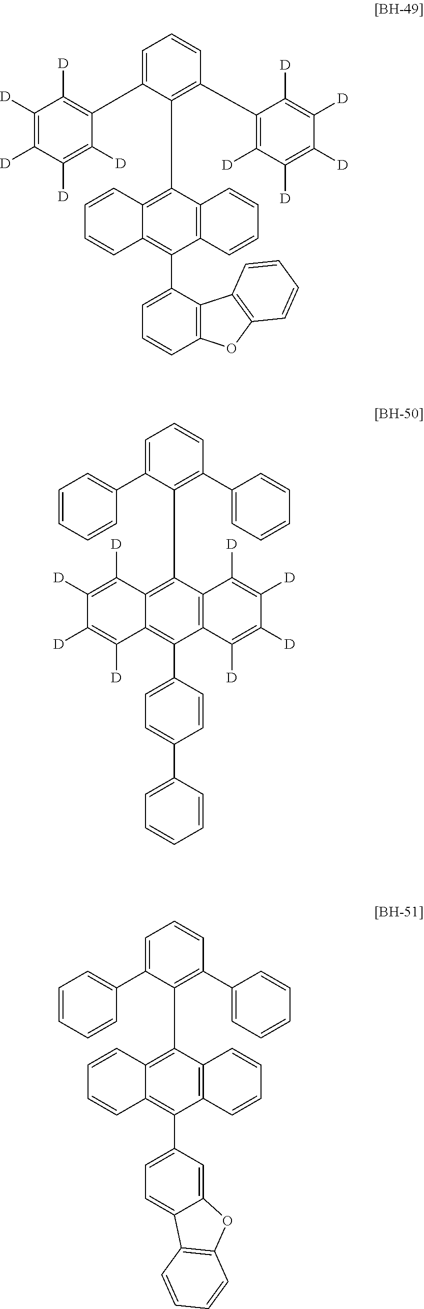 [SMILES: [2H]c1c([2H])c([2H])c(-c2cccc(-c3c([2H])c([2H])c([2H])c([2H])c3[2H])c2-c2c3ccccc3c(-c3cccc4oc5ccccc5c34)c3ccccc23)c([2H])c1[2H] ; [2H]c1c([2H])c([2H])c2c(-c3c(-c4ccccc4)cccc3-c3ccccc3)c3c([2H])c([2H])c([2H])c([2H])c3c(-c3ccc(-c4ccccc4)cc3)c2c1[2H] ; c1ccc(-c2cccc(-c3ccccc3)c2-c2c3ccccc3c(-c3ccc4c(c3)oc3ccccc34)c3ccccc23)cc1]

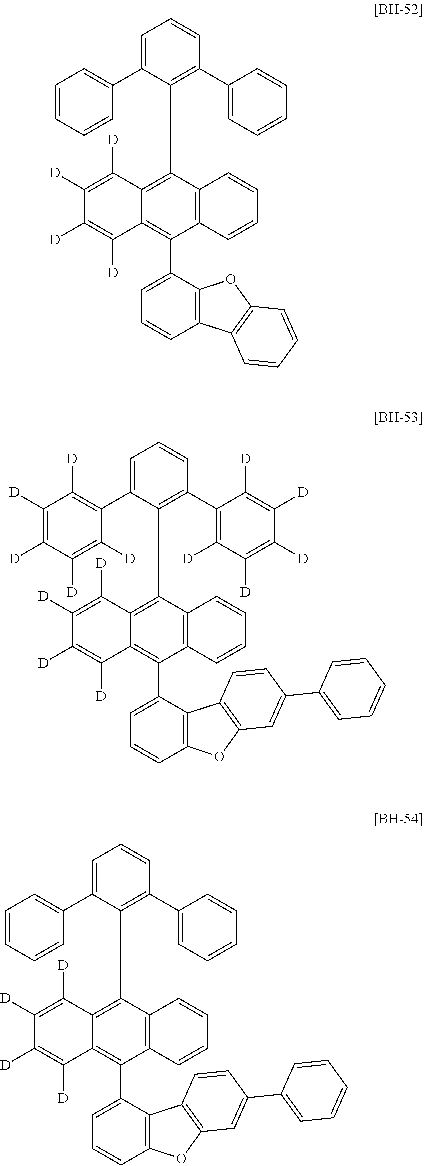 [SMILES: [2H]c1c([2H])c([2H])c2c(-c3cccc4c3oc3ccccc34)c3ccccc3c(-c3c(-c4ccccc4)cccc3-c3ccccc3)c2c1[2H] ; [2H]c1c([2H])c([2H])c(-c2cccc(-c3c([2H])c([2H])c([2H])c([2H])c3[2H])c2-c2c3ccccc3c(-c3cccc4oc5cc(-c6ccccc6)ccc5c34)c3c([2H])c([2H])c([2H])c([2H])c23)c([2H])c1[2H] ; [2H]c1c([2H])c([2H])c2c(-c3cccc4oc5cc(-c6ccccc6)ccc5c34)c3ccccc3c(-c3c(-c4ccccc4)cccc3-c3ccccc3)c2c1[2H]]

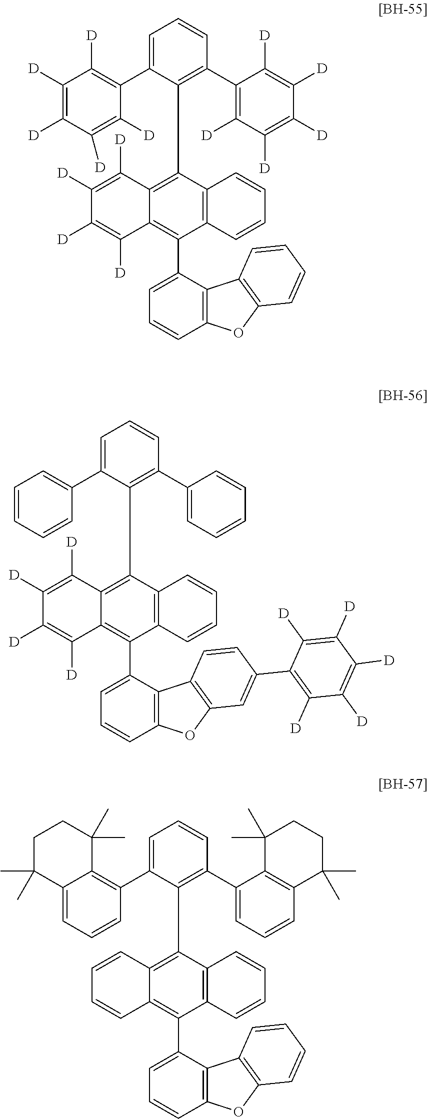

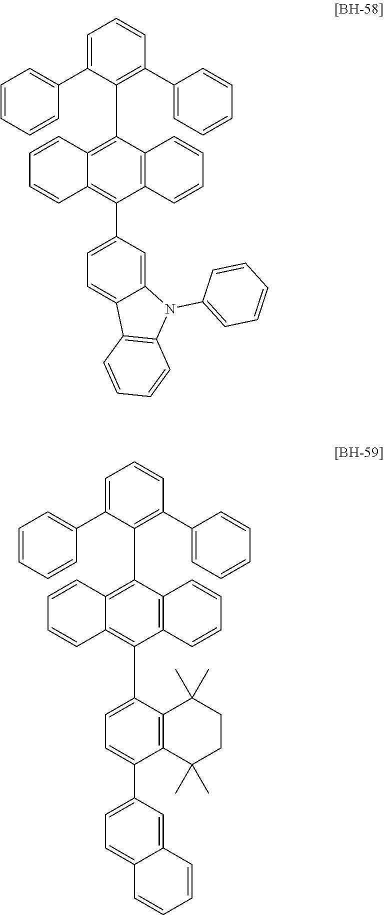

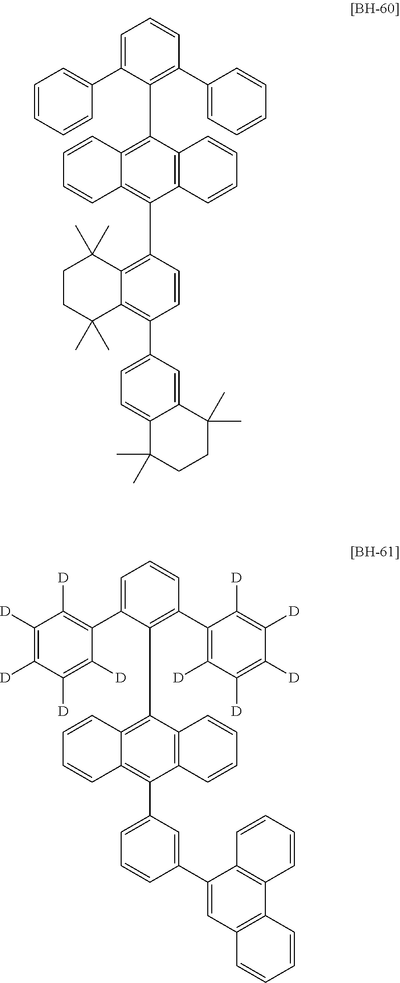

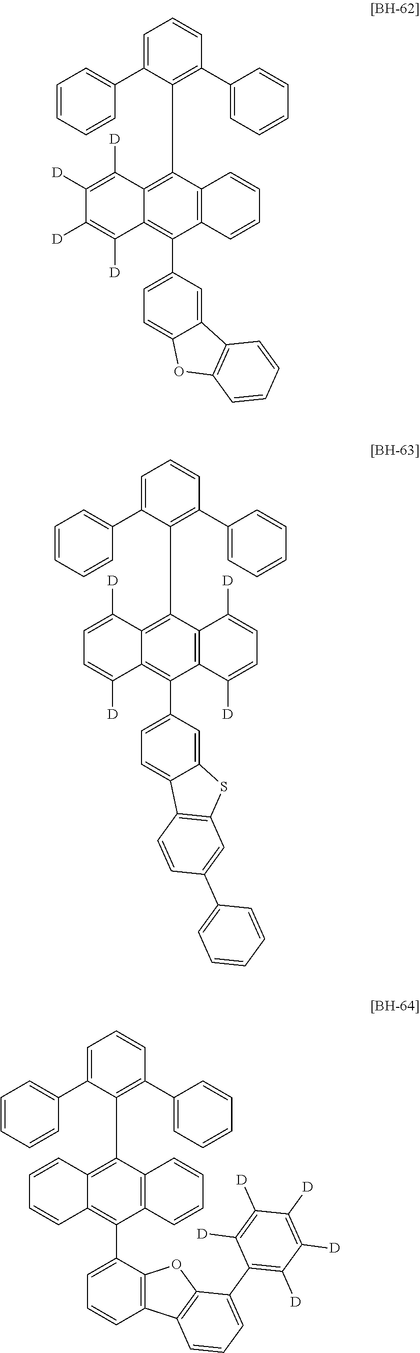 [SMILES: [2H]c1c([2H])c([2H])c2c(-c3c(-c4ccccc4)cccc3-c3ccccc3)c3ccccc3c(-c3ccc4oc5ccccc5c4c3)c2c1[2H] ; [2H]c1ccc([2H])c2c(-c3c(-c4ccccc4)cccc3-c3ccccc3)c3c([2H])ccc([2H])c3c(-c3ccc4c(c3)sc3cc(-c5ccccc5)ccc34)c12 ; [2H]c1c([2H])c([2H])c(-c2cccc3c2oc2c(-c4c5ccccc5c(-c5c(-c6ccccc6)cccc5-c5ccccc5)c5ccccc45)cccc23)c([2H])c1[2H]]

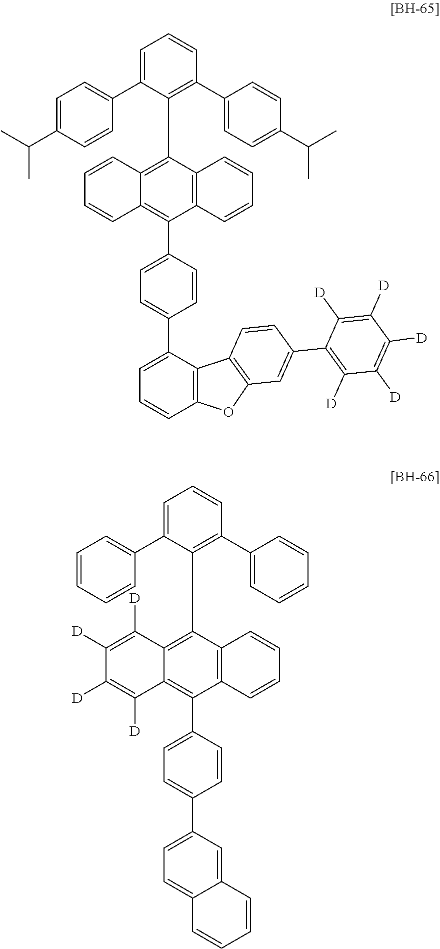 [SMILES: [2H]c1c([2H])c([2H])c(-c2ccc3c(c2)oc2cccc(-c4ccc(-c5c6ccccc6c(-c6c(-c7ccc(C(C)C)cc7)cccc6-c6ccc(C(C)C)cc6)c6ccccc56)cc4)c23)c([2H])c1[2H] ; [2H]c1c([2H])c([2H])c2c(-c3c(-c4ccccc4)cccc3-c3ccccc3)c3ccccc3c(-c3ccc(-c4ccc5ccccc5c4)cc3)c2c1[2H]]

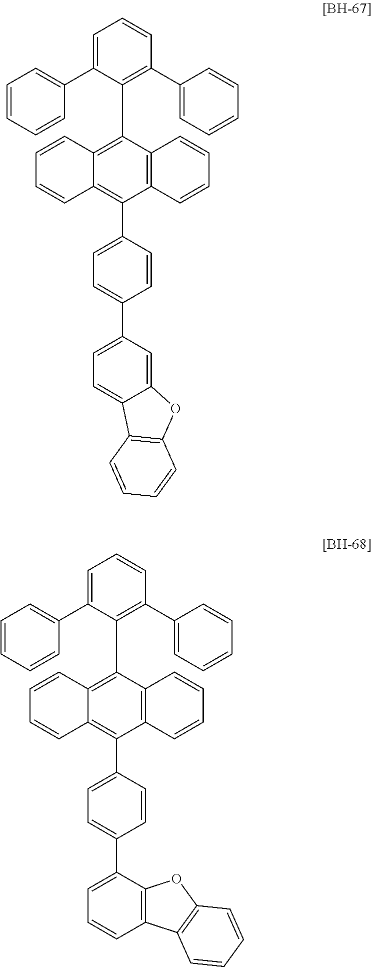 [SMILES: c1ccc(-c2cccc(-c3ccccc3)c2-c2c3ccccc3c(-c3ccc(-c4ccc5c(c4)oc4ccccc45)cc3)c3ccccc23)cc1 ; c1ccc(-c2cccc(-c3ccccc3)c2-c2c3ccccc3c(-c3ccc(-c4cccc5c4oc4ccccc45)cc3)c3ccccc23)cc1]

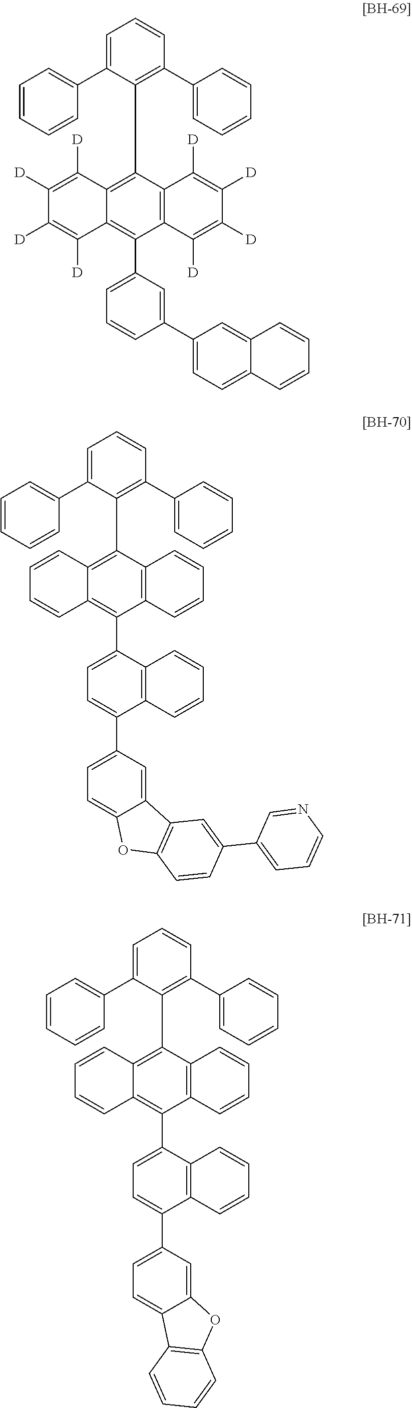 [SMILES: [2H]c1c([2H])c([2H])c2c(-c3c(-c4ccccc4)cccc3-c3ccccc3)c3c([2H])c([2H])c([2H])c([2H])c3c(-c3cccc(-c4ccc5ccccc5c4)c3)c2c1[2H] ; c1ccc(-c2cccc(-c3ccccc3)c2-c2c3ccccc3c(-c3ccc(-c4ccc5oc6ccc(-c7cccnc7)cc6c5c4)c4ccccc34)c3ccccc23)cc1 ; c1ccc(-c2cccc(-c3ccccc3)c2-c2c3ccccc3c(-c3ccc(-c4ccc5c(c4)oc4ccccc45)c4ccccc34)c3ccccc23)cc1]

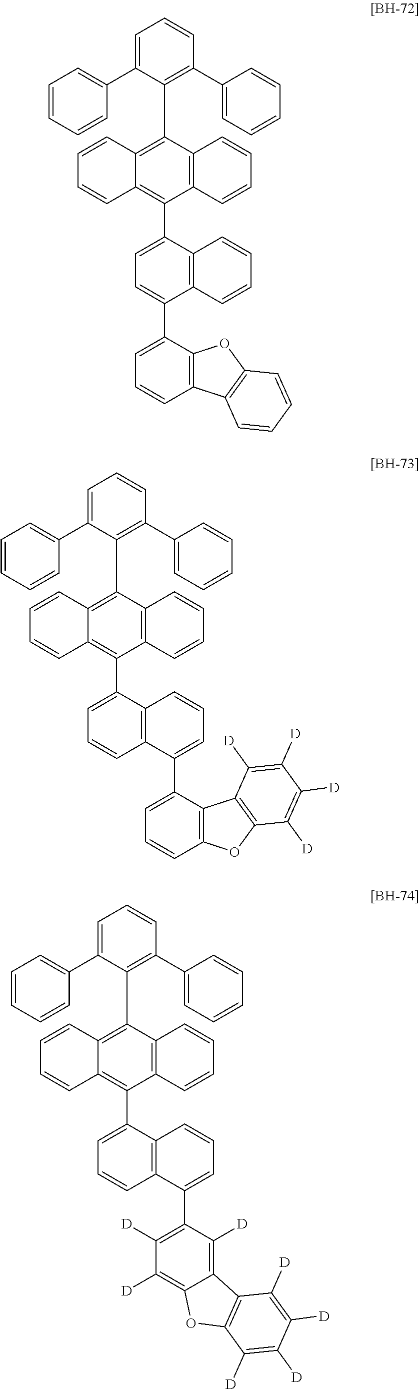 [SMILES: c1ccc(-c2cccc(-c3ccccc3)c2-c2c3ccccc3c(-c3ccc(-c4cccc5c4oc4ccccc45)c4ccccc34)c3ccccc23)cc1 ; [2H]c1c([2H])c([2H])c2c(oc3cccc(-c4cccc5c(-c6c7ccccc7c(-c7c(-c8ccccc8)cccc7-c7ccccc7)c7ccccc67)cccc45)c32)c1[2H] ; [2H]c1c([2H])c([2H])c2c(oc3c([2H])c([2H])c(-c4cccc5c(-c6c7ccccc7c(-c7c(-c8ccccc8)cccc7-c7ccccc7)c7ccccc67)cccc45)c([2H])c32)c1[2H]]

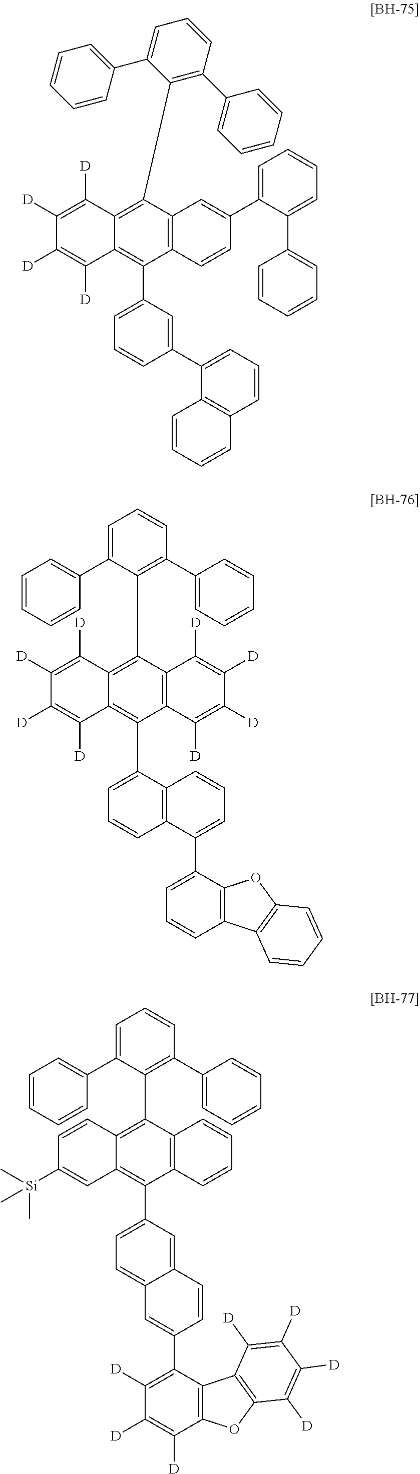 [SMILES: [2H]c1c([2H])c([2H])c2c(-c3c(-c4ccccc4)cccc3-c3ccccc3)c3cc(-c4ccccc4-c4ccccc4)ccc3c(-c3cccc(-c4cccc5ccccc45)c3)c2c1[2H] ; [2H]c1c([2H])c([2H])c2c(-c3cccc4c(-c5cccc6c5oc5ccccc56)cccc34)c3c([2H])c([2H])c([2H])c([2H])c3c(-c3c(-c4ccccc4)cccc3-c3ccccc3)c2c1[2H] ; [2H]c1c([2H])c([2H])c2c(oc3c([2H])c([2H])c([2H])c(-c4ccc5cc(-c6c7ccccc7c(-c7c(-c8ccccc8)cccc7-c7ccccc7)c7ccc([Si](C)(C)C)cc67)ccc5c4)c32)c1[2H]]

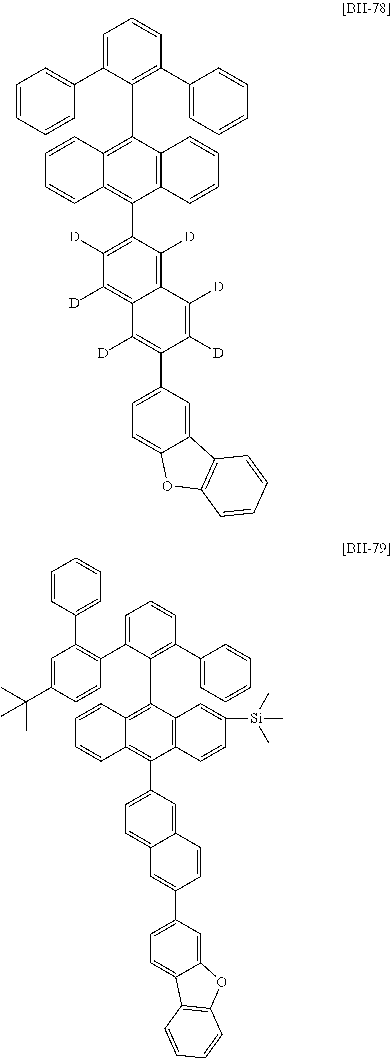 [SMILES: [2H]c1c(-c2c3ccccc3c(-c3c(-c4ccccc4)cccc3-c3ccccc3)c3ccccc23)c([2H])c2c([2H])c([2H])c(-c3ccc4oc5ccccc5c4c3)c([2H])c2c1[2H] ; CC(C)(C)c1ccc(-c2cccc(-c3ccccc3)c2-c2c3ccccc3c(-c3ccc4cc(-c5ccc6c(c5)oc5ccccc56)ccc4c3)c3ccc([Si](C)(C)C)cc23)c(-c2ccccc2)c1]

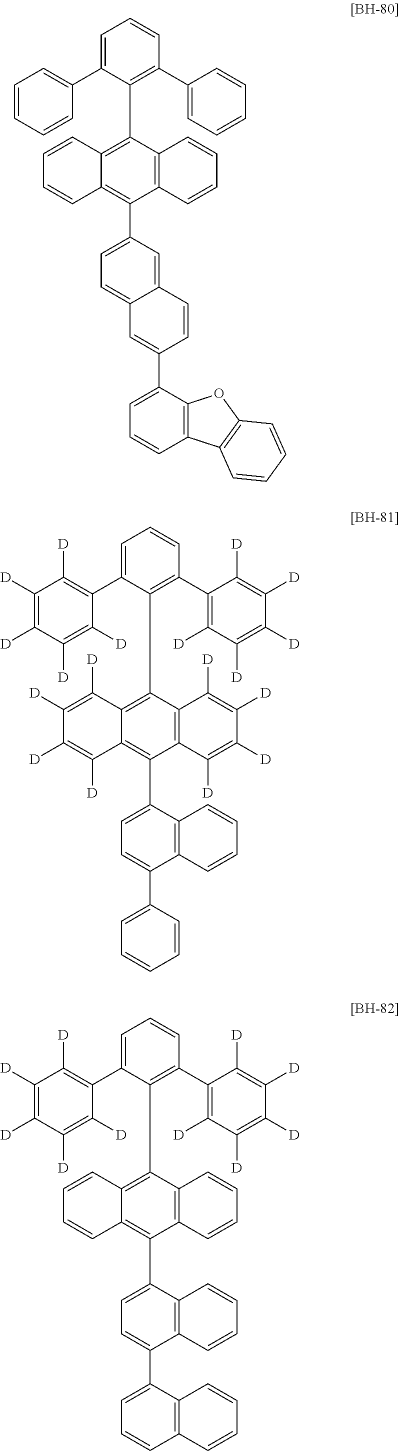 [SMILES: c1ccc(-c2cccc(-c3ccccc3)c2-c2c3ccccc3c(-c3ccc4cc(-c5cccc6c5oc5ccccc56)ccc4c3)c3ccccc23)cc1 ; [2H]c1c([2H])c([2H])c(-c2cccc(-c3c([2H])c([2H])c([2H])c([2H])c3[2H])c2-c2c3c([2H])c([2H])c([2H])c([2H])c3c(-c3ccc(-c4ccccc4)c4ccccc34)c3c([2H])c([2H])c([2H])c([2H])c23)c([2H])c1[2H] ; [2H]c1c([2H])c([2H])c(-c2cccc(-c3c([2H])c([2H])c([2H])c([2H])c3[2H])c2-c2c3ccccc3c(-c3ccc(-c4cccc5ccccc45)c4ccccc34)c3ccccc23)c([2H])c1[2H]]

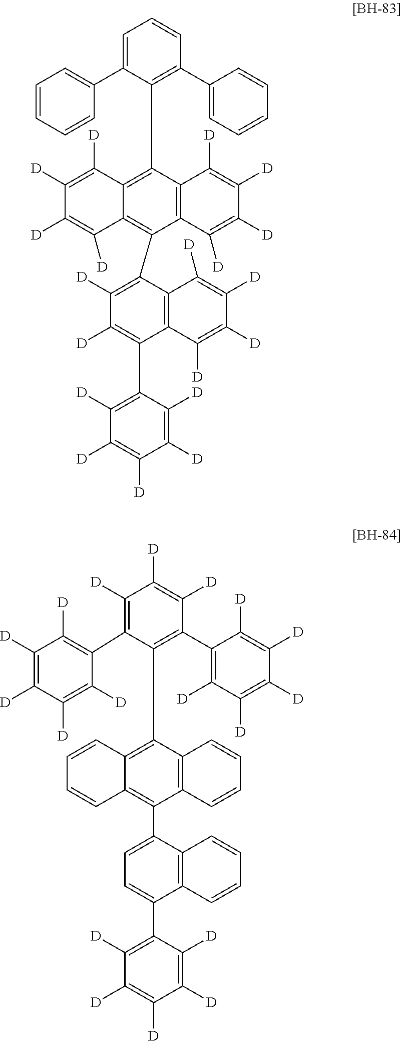 [SMILES: [2H]c1c([2H])c([2H])c(-c2c([2H])c([2H])c(-c3c4c([2H])c([2H])c([2H])c([2H])c4c(-c4c(-c5ccccc5)cccc4-c4ccccc4)c4c([2H])c([2H])c([2H])c([2H])c34)c3c([2H])c([2H])c([2H])c([2H])c23)c([2H])c1[2H] ; [2H]c1c([2H])c([2H])c(-c2c([2H])c([2H])c([2H])c(-c3c([2H])c([2H])c([2H])c([2H])c3[2H])c2-c2c3ccccc3c(-c3ccc(-c4c([2H])c([2H])c([2H])c([2H])c4[2H])c4ccccc34)c3ccccc23)c([2H])c1[2H]]

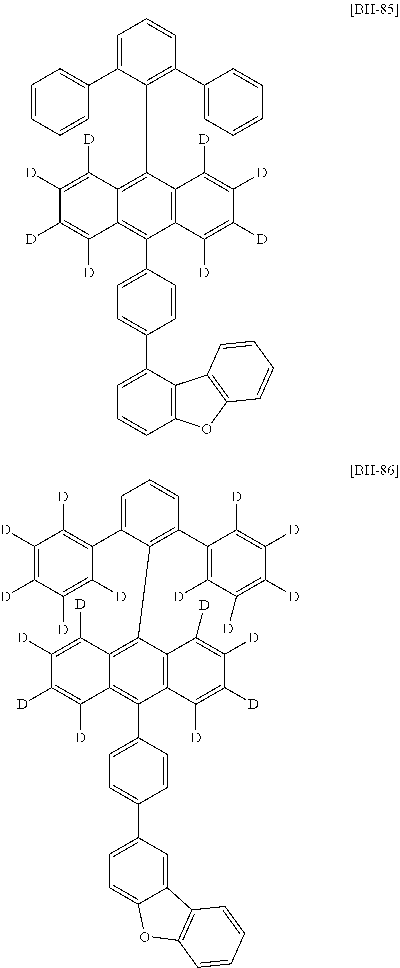

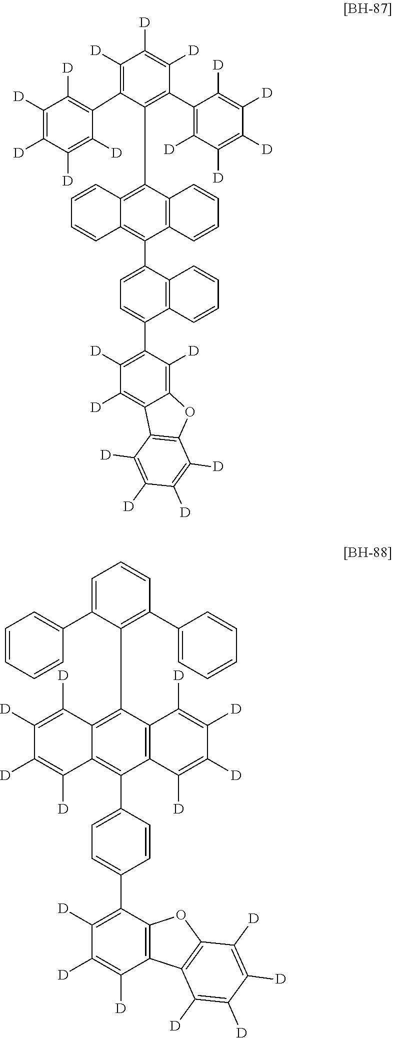 [SMILES: [2H]c1c([2H])c([2H])c(-c2c([2H])c([2H])c([2H])c(-c3c([2H])c([2H])c([2H])c([2H])c3[2H])c2-c2c3ccccc3c(-c3ccc(-c4c([2H])c([2H])c5c(oc6c([2H])c([2H])c([2H])c([2H])c65)c4[2H])c4ccccc34)c3ccccc23)c([2H])c1[2H] ; [2H]c1c([2H])c([2H])c2c(oc3c(-c4ccc(-c5c6c([2H])c([2H])c([2H])c([2H])c6c(-c6c(-c7ccccc7)cccc6-c6ccccc6)c6c([2H])c([2H])c([2H])c([2H])c56)cc4)c([2H])c([2H])c([2H])c32)c1[2H]]

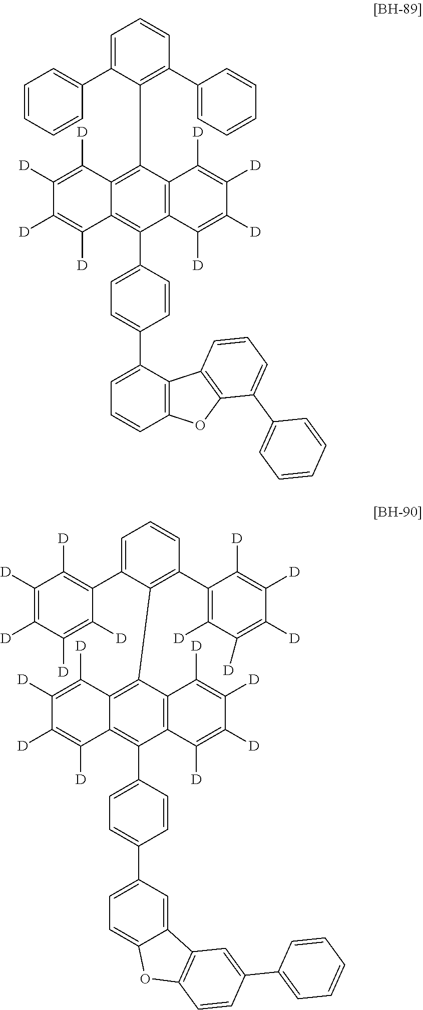 [SMILES: [2H]c1c([2H])c([2H])c2c(-c3c(-c4ccccc4)cccc3-c3ccccc3)c3c([2H])c([2H])c([2H])c([2H])c3c(-c3ccc(-c4cccc5oc6c(-c7ccccc7)cccc6c45)cc3)c2c1[2H] ; [2H]c1c([2H])c([2H])c(-c2cccc(-c3c([2H])c([2H])c([2H])c([2H])c3[2H])c2-c2c3c([2H])c([2H])c([2H])c([2H])c3c(-c3ccc(-c4ccc5oc6ccc(-c7ccccc7)cc6c5c4)cc3)c3c([2H])c([2H])c([2H])c([2H])c23)c([2H])c1[2H]]

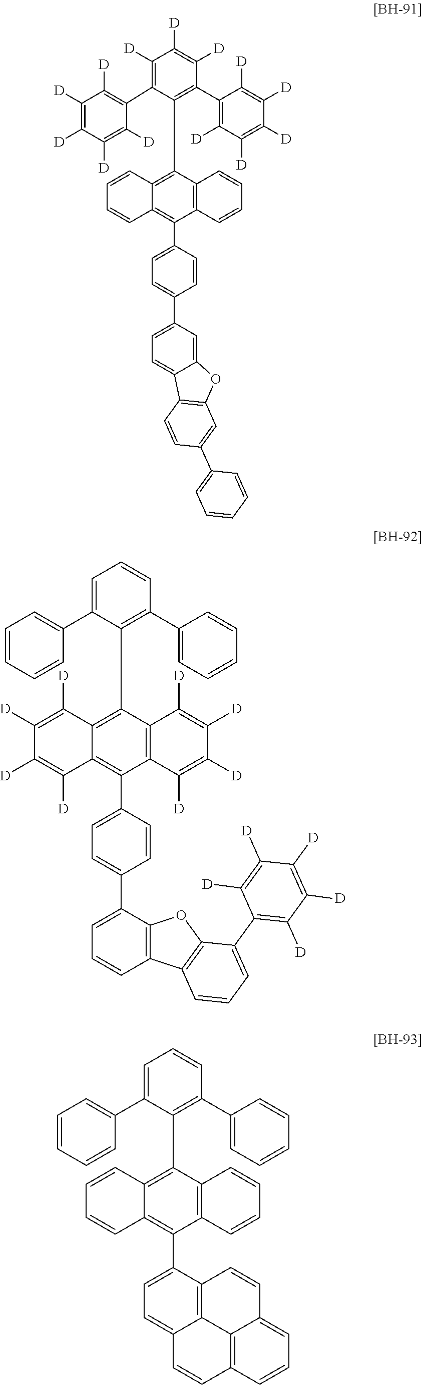

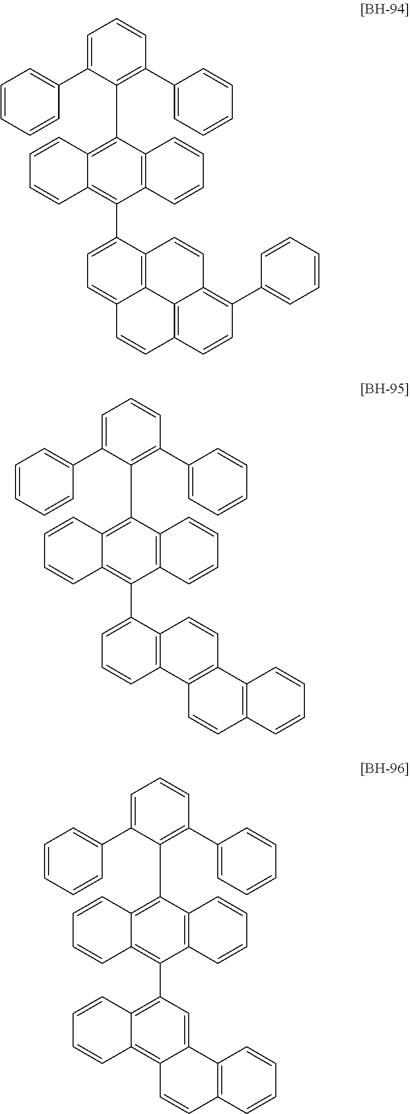

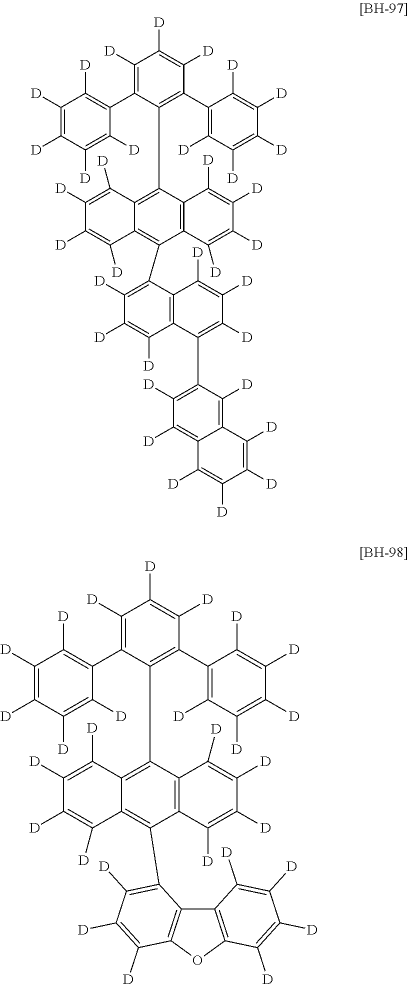

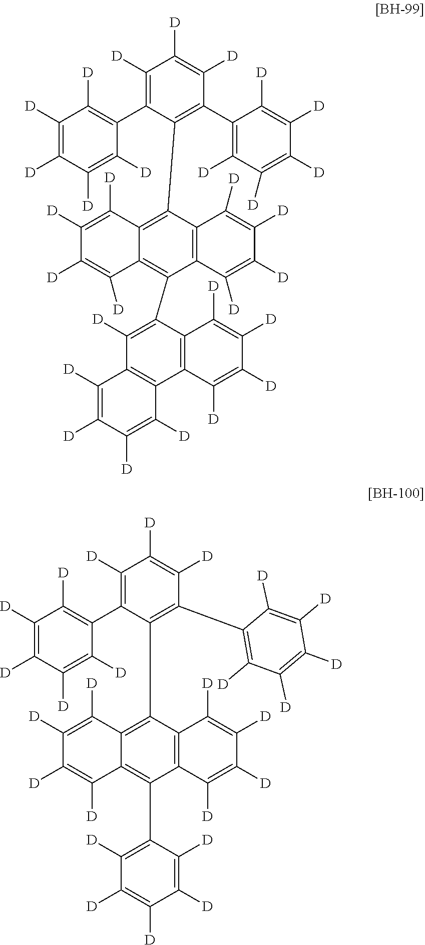 [SMILES: [2H]c1c([2H])c([2H])c(-c2c([2H])c([2H])c([2H])c(-c3c([2H])c([2H])c([2H])c([2H])c3[2H])c2-c2c3c([2H])c([2H])c([2H])c([2H])c3c(-c3c([2H])c4c([2H])c([2H])c([2H])c([2H])c4c4c([2H])c([2H])c([2H])c([2H])c34)c3c([2H])c([2H])c([2H])c([2H])c23)c([2H])c1[2H] ; [2H]c1c([2H])c([2H])c(-c2c([2H])c([2H])c([2H])c(-c3c([2H])c([2H])c([2H])c([2H])c3[2H])c2-c2c3c([2H])c([2H])c([2H])c([2H])c3c(-c3c([2H])c([2H])c([2H])c([2H])c3[2H])c3c([2H])c([2H])c([2H])c([2H])c23)c([2H])c1[2H]]

10. The organic light-emitting diode of claim 1 or 2, wherein the organic light-emitting diode comprises at least one of a hole injection layer, a hole transport layer, a functional layer capable of both hole injection and hole transport, an electron transport layer, and an electron injection layer, in addition to the light-emitting layer.

11. The organic light-emitting diode of claim 1 or 2, wherein the dopant compound in the light-emitting layer is a polycyclic compound represented by any one of the following [BD-1] to [BD-48]:

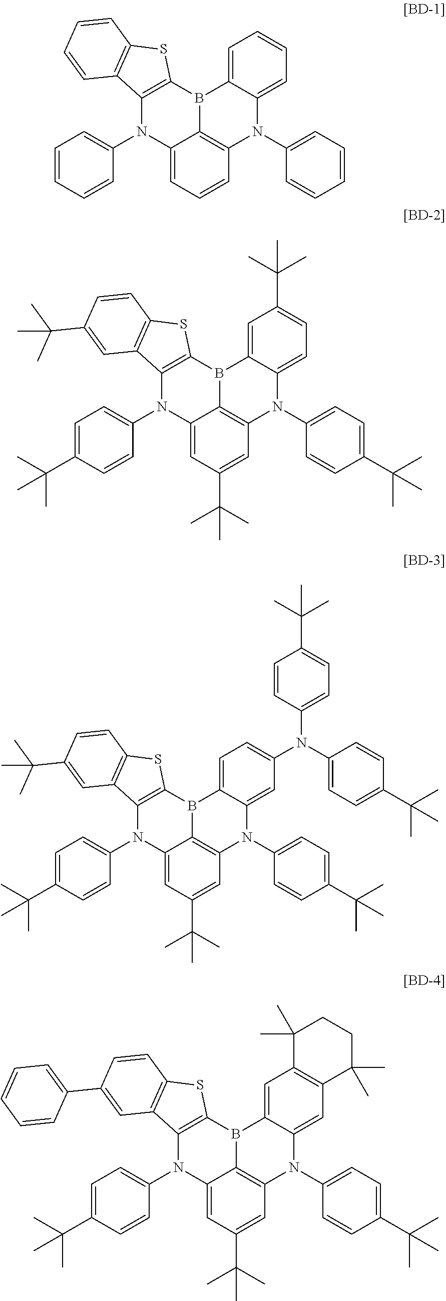

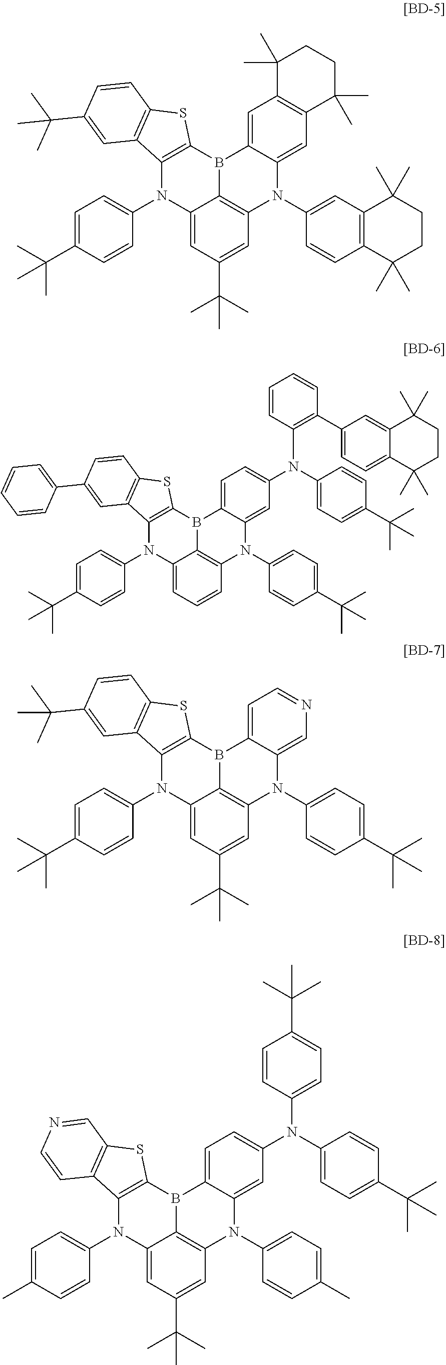

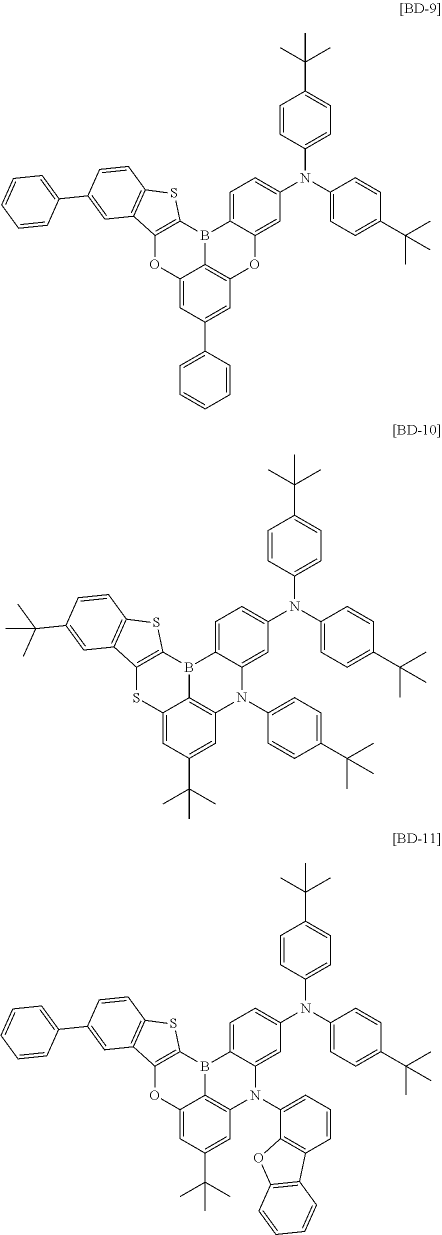

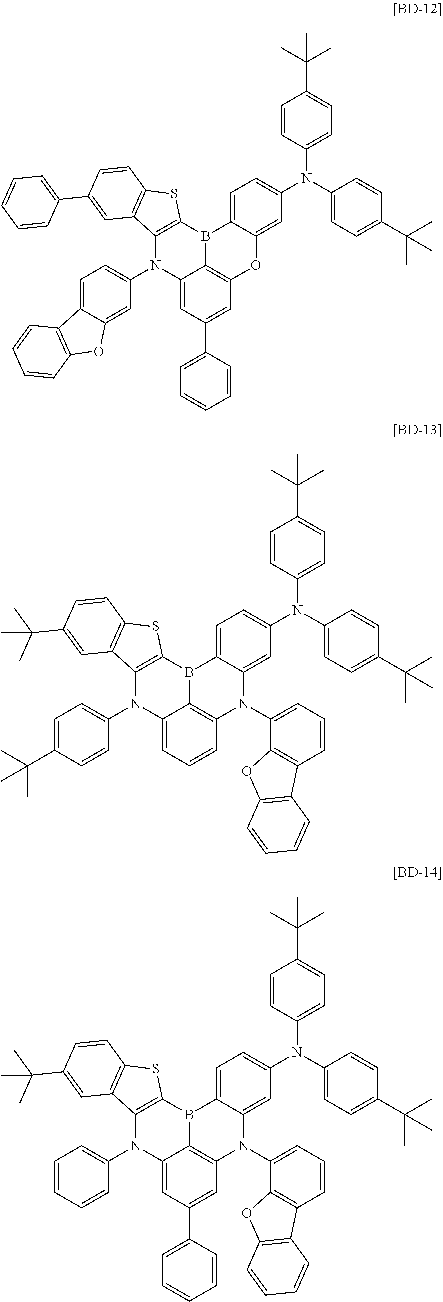

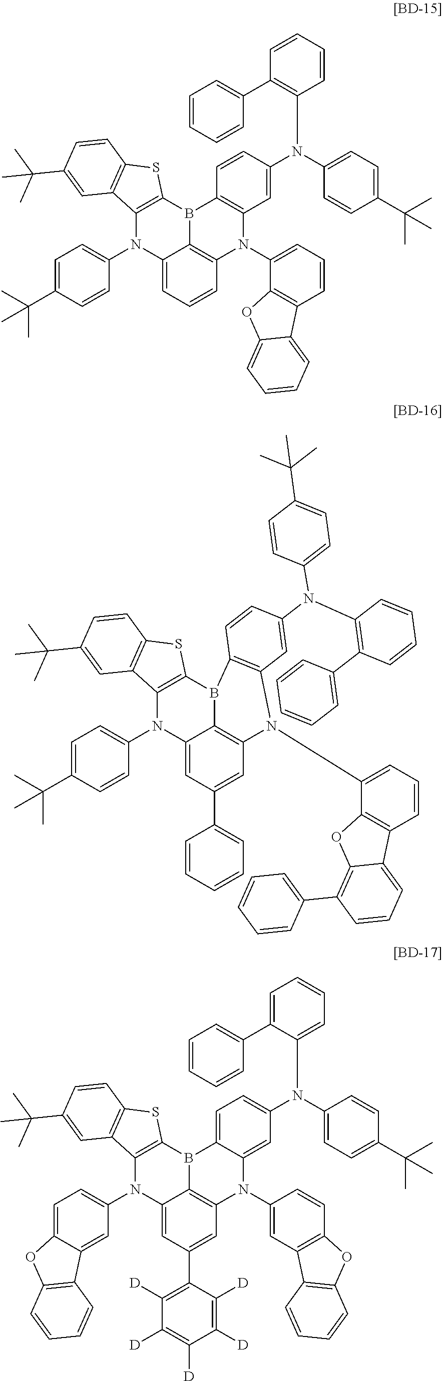

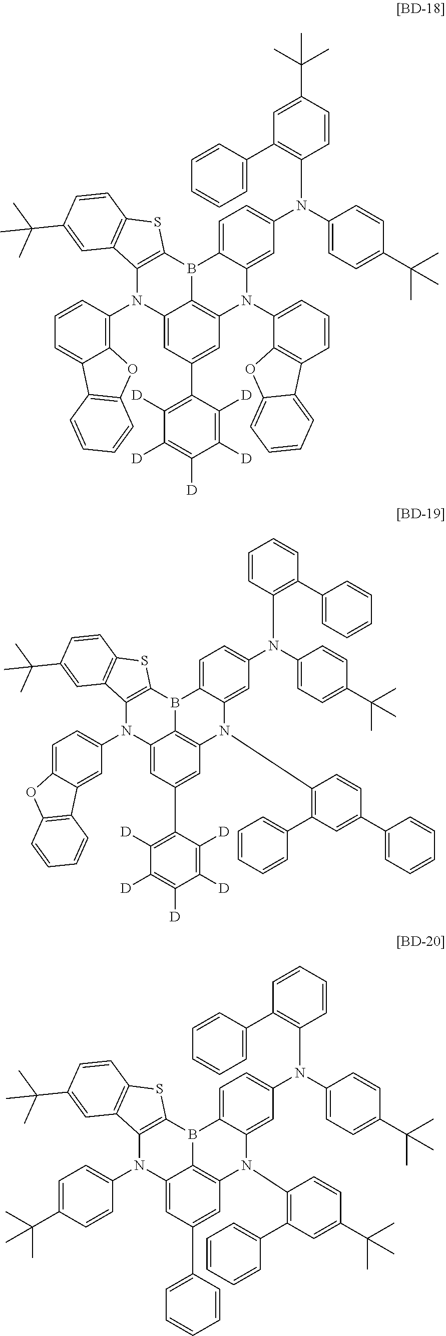

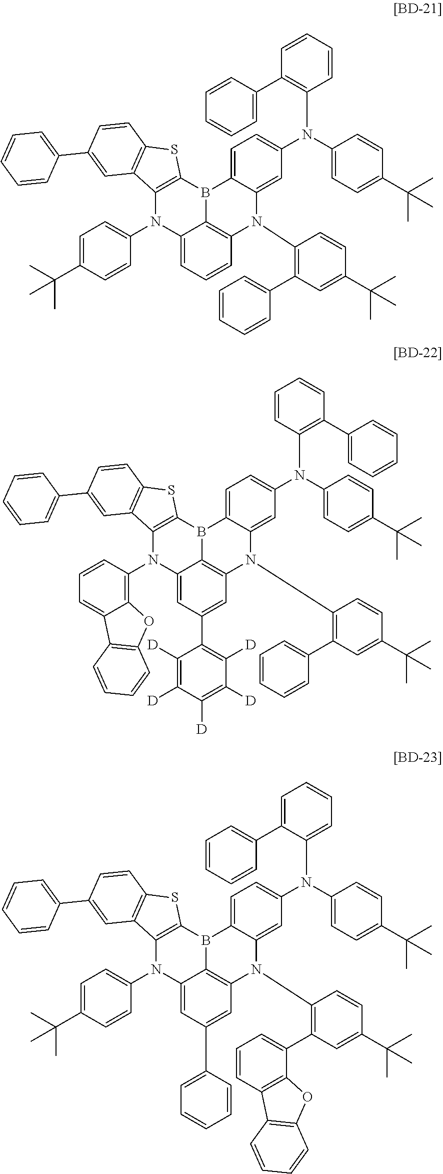 [SMILES: CC(C)(C)c1ccc(N(c2ccc3c(c2)N(c2ccc(C(C)(C)C)cc2-c2ccccc2)c2cccc4c2B3c2sc3ccc(-c5ccccc5)cc3c2N4c2ccc(C(C)(C)C)cc2)c2ccccc2-c2ccccc2)cc1 ; [2H]c1c([2H])c([2H])c(-c2cc3c4c(c2)N(c2cccc5c2oc2ccccc25)c2c(sc5ccc(-c6ccccc6)cc25)B4c2ccc(N(c4ccc(C(C)(C)C)cc4)c4ccccc4-c4ccccc4)cc2N3c2ccc(C(C)(C)C)cc2-c2ccccc2)c([2H])c1[2H] ; CC(C)(C)c1ccc(N(c2ccc3c(c2)N(c2ccc(C(C)(C)C)cc2-c2cccc4c2oc2ccccc24)c2cc(-c4ccccc4)cc4c2B3c2sc3ccc(-c5ccccc5)cc3c2N4c2ccc(C(C)(C)C)cc2)c2ccccc2-c2ccccc2)cc1]

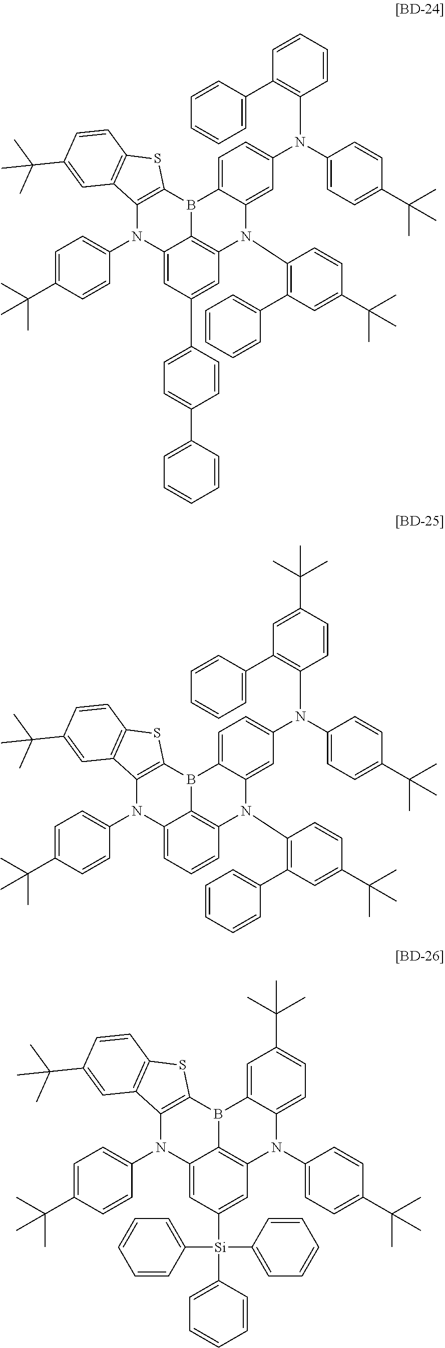

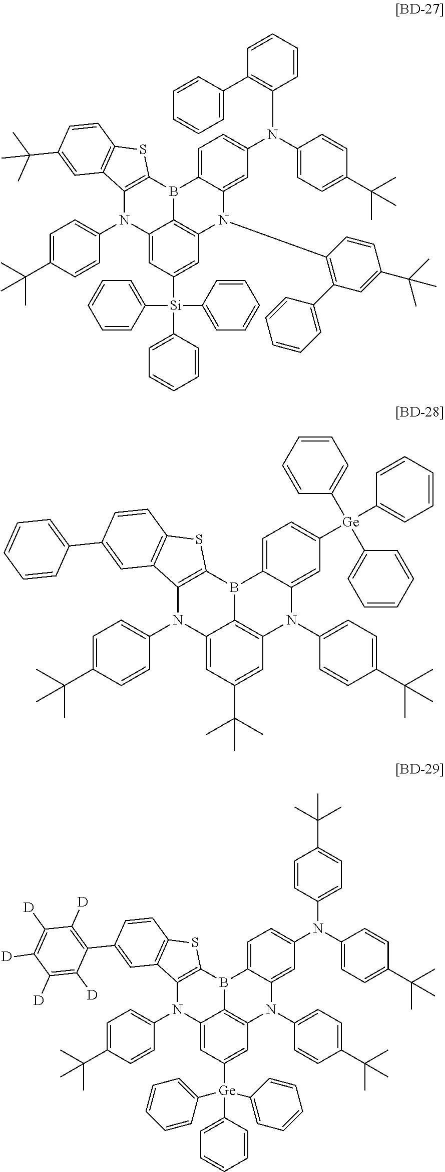

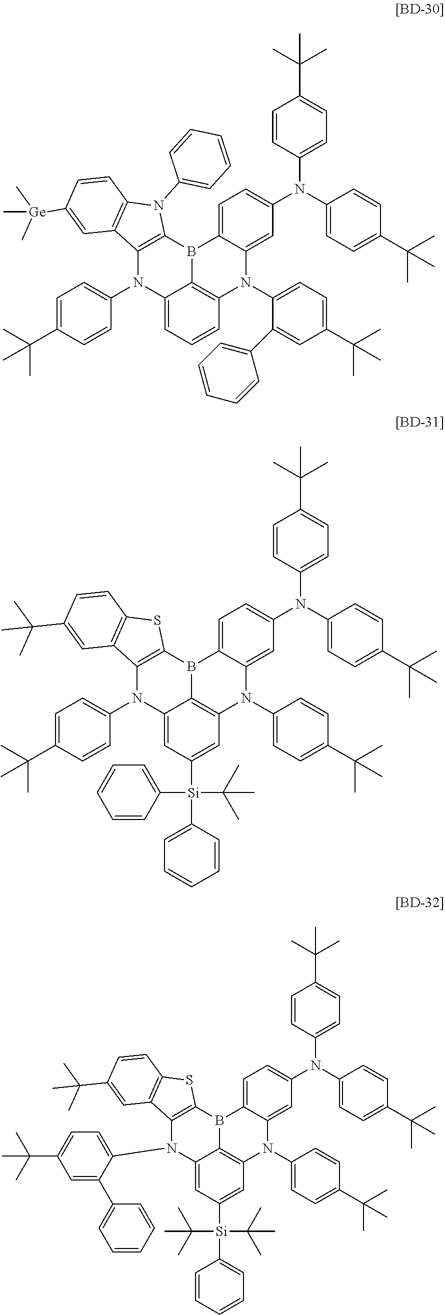

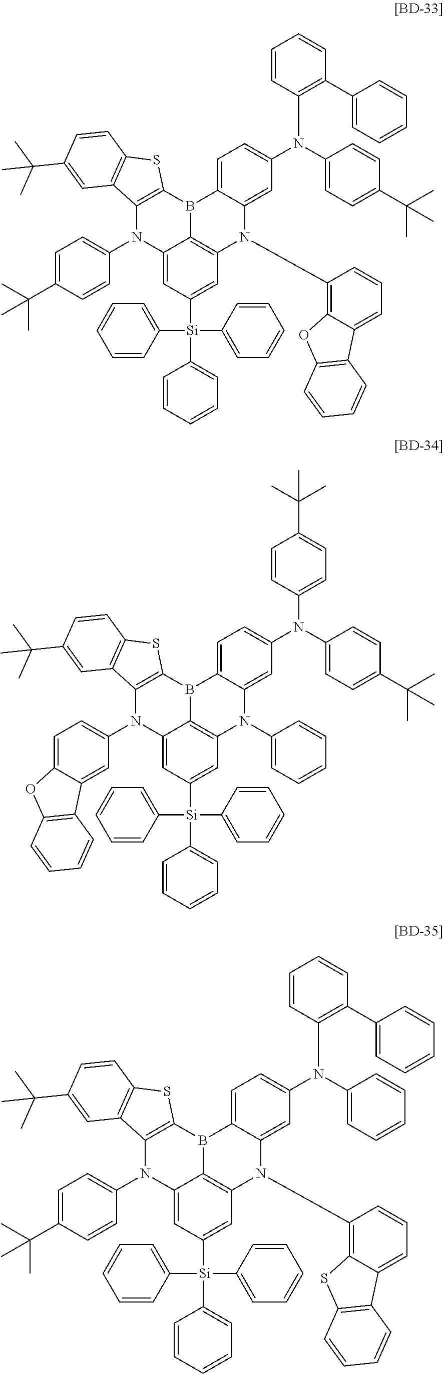

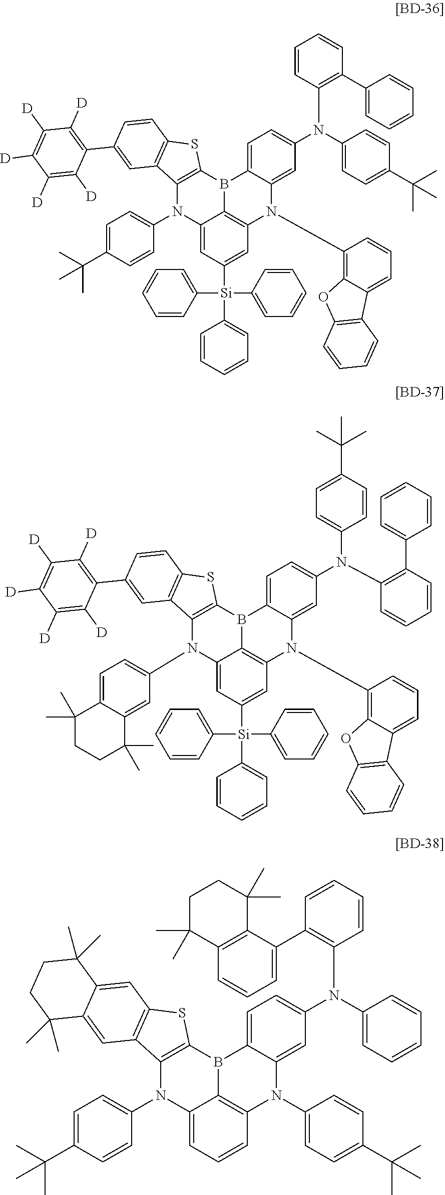

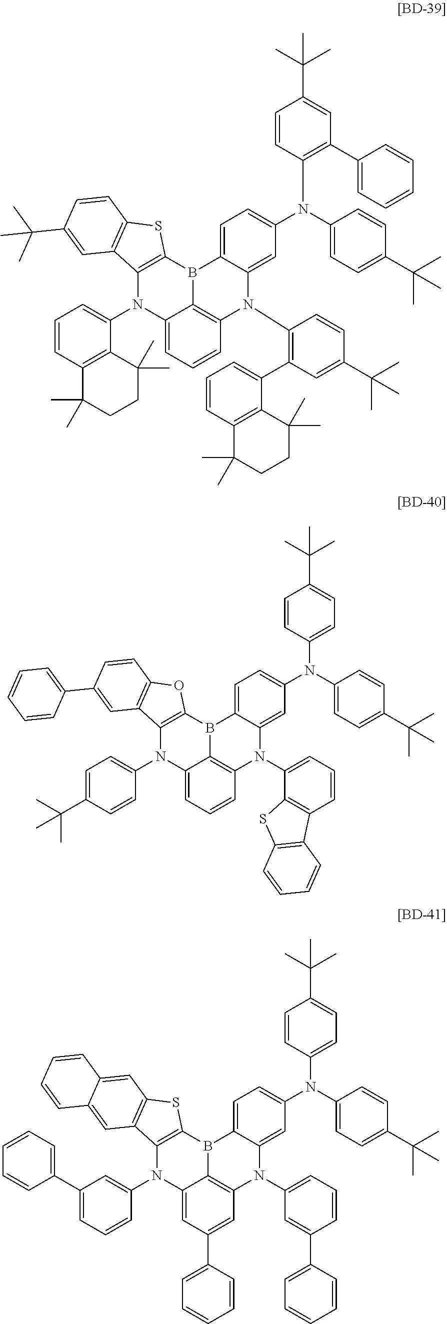 [SMILES: CC(C)(C)c1ccc(N(c2ccc3c(c2)N(c2ccc(C(C)(C)C)cc2-c2cccc4c2C(C)(C)CCC4(C)C)c2cccc4c2B3c2sc3ccc(C(C)(C)C)cc3c2N4c2cccc3c2C(C)(C)CCC3(C)C)c2ccc(C(C)(C)C)cc2-c2ccccc2)cc1 ; CC(C)(C)c1ccc(N(c2ccc(C(C)(C)C)cc2)c2ccc3c(c2)N(c2cccc4c2sc2ccccc24)c2cccc4c2B3c2oc3ccc(-c5ccccc5)cc3c2N4c2ccc(C(C)(C)C)cc2)cc1 ; CC(C)(C)c1ccc(N(c2ccc(C(C)(C)C)cc2)c2ccc3c(c2)N(c2cccc(-c4ccccc4)c2)c2cc(-c4ccccc4)cc4c2B3c2sc3cc5ccccc5cc3c2N4c2cccc(-c3ccccc3)c2)cc1]

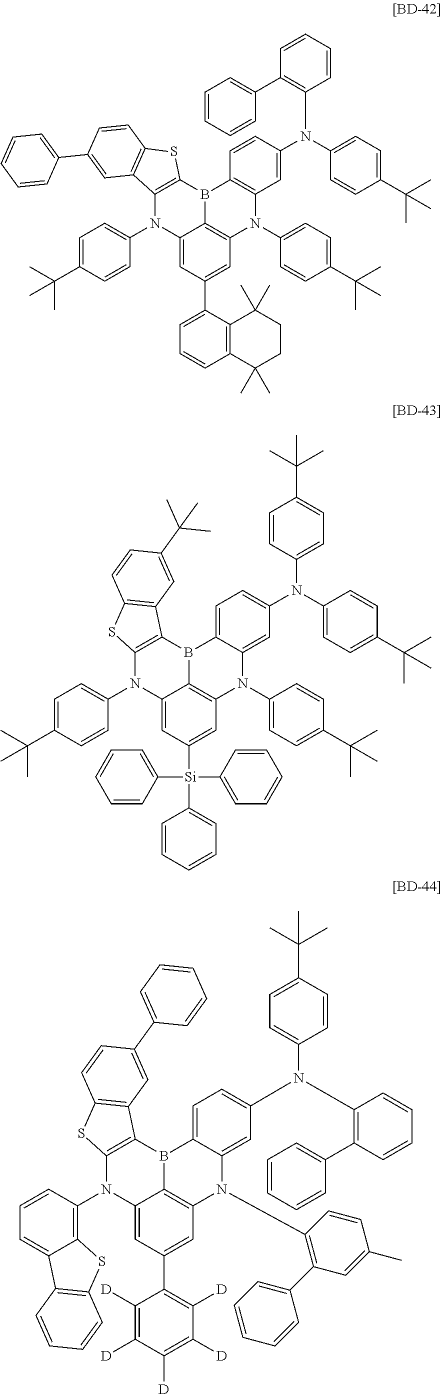

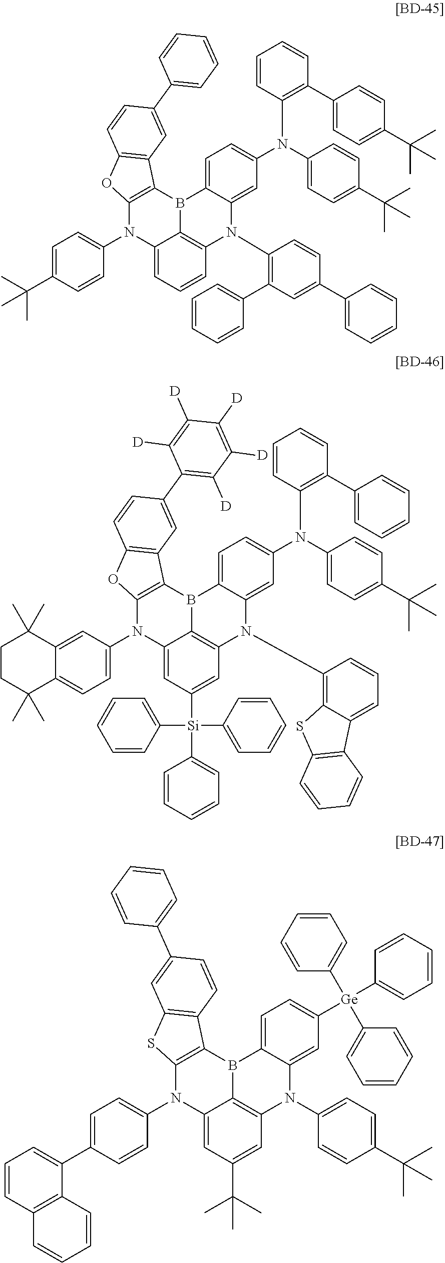

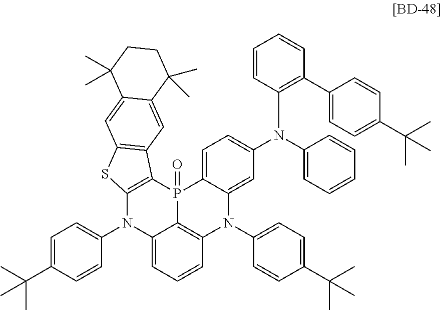 [SMILES: CC(C)(C)c1ccc(-c2ccccc2N(c2ccccc2)c2ccc3c(c2)N(c2ccc(C(C)(C)C)cc2)c2cccc4c2P3(=O)c2c(sc3cc5c(cc23)C(C)(C)CCC5(C)C)N4c2ccc(C(C)(C)C)cc2)cc1]

12. The organic light-emitting diode of claim 10, wherein at least one selected form the layers is formed by a deposition process or solution process.

13. The organic light-emitting diode of claim 1 or 2, wherein the dopant in the light-emitting layer comprises two or more different dopant compounds including: the compound represented by any one of Chemical Formulas 3, 4, and 3-1 to 4-3; and at least one species of dopant compounds other than the dopant compounds represented by the Chemical Formulas 3, 4, and 3-1 to 4-3, the different dopant compounds being mixed or stacked.

14. The organic light-emitting diode of claim 1 or 2, wherein the host in the light-emitting layer comprises two or more different host compounds including: the anthracene compound represented by any one of Chemical Formula A; and at least one species of host compounds other than the anthracene compound, the different host compounds being mixed or stacked.

15. The organic light-emitting diode of claim 1, wherein the organic light-emitting diode is used in any one selected from flat display devices, flexible display devices, monochrome or grayscale flat illumination devices, monochrome or grayscale flexible illumination devices, vehicle display devices, and display devices for virtual or augmented reality.
